(19) United States
(12) Patent Application Publication
(10) Pub. No.: US 2026/0207710 A1
(43) Pub. Date: Jul. 23, 2026

(54) CANCER THERAPY WITH IL-2 CONJUGATES AND CHIMERIC ANTIGEN RECEPTOR THERAPIES

(71) Applicant: Synthorx, Inc., Bridgewater, NJ (US)

(72) Inventors: Giovanni ABBADESSA, Winchester, MA (US); Carolina E. CAFFARO, San Diego, CA (US); Paula FRAENKEL, Cambridge, MA (US); Xiangming LI, Cambridge, MA (US); Marcos MILLA, Carlsbad, CA (US); Jerod PTACIN, San Diego, CA (US)

(73) Assignee: Synthorx, Inc., Bridgewater, NJ (US)

(21) Appl. No.: 19/137,781

(22) PCT Filed: Dec. 20, 2023
(86) PCT No.: PCT/US2023/085227

§ 371 Date: Jun. 11, 2025

(30) Foreign Application Priority Data
Dec. 21, 2022 (WO) PCT/US2022/082151

(51) Int. Cl. A61K 38/20; A61K 9/00; A61K 31/138; A61K 31/167; A61K 35/17; A61K 38/17; A61K 40/11; A61K 40/15; A61K 40/31; A61K 40/42; A61P 35/02

(52) U.S. Cl. CPC A61K 38/2013; A61K 9/0019; A61K 31/138; A61K 31/167; A61K 35/17; A61K 38/1774; A61K 40/11; A61K 40/15; A61K 40/31; A61K 40/4211; A61K 40/4212; A61K 40/4215; A61P 35/02; A61K 2239/27; A61K 2239/48

(57) ABSTRACT

Disclosed herein are methods for treating a cancer in a subject in need thereof, comprising administering (a) an IL-2 conjugate, and (b) a chimeric antigen receptor (CAR) therapy. Also disclosed herein are methods for treating diffuse large B-cell lymphoma (DLBCL) in a subject in need thereof, comprising administering an IL-2 conjugate.

### CROSS-REFERENCE TO RELATED APPLICATIONS

This application claims the benefit of priority of International Application No. PCT/US2022/082151, filed Dec. 21, 2022, the content of which is incorporated by reference herein for all purposes.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+ T and B lymphocytes, monocytes, macrophages, endothelial cells, and other tissue residents.

In some instances, interleukin 2 (IL-2) signaling is used to modulate T cell responses and subsequently for treatment of a cancer. Accordingly, in one aspect, provided herein are methods of treating a cancer in a subject in need thereof, comprising administering to a subject (a) an IL-2 conjugate, and (b) a chimeric antigen receptor (CAR) therapy. In another aspect, provided herein are methods of treating diffuse large B-cell lymphoma (DLBCL) in a subject comprising administering an IL-2 conjugate.

### SUMMARY OF THE DISCLOSURE

Described herein are methods of treating a cancer in a subject in need thereof, comprising administering to a subject (a) an IL-2 conjugate, and (b) a CAR therapy, wherein the IL-2 conjugate comprises the amino acid sequence of SEQ ID NO: 1 having an unnatural amino acid residue described herein at position 64, e.g., the amino acid sequence of SEQ ID NO: 2.

Also described herein are methods of treating diffuse large B-cell lymphoma (DLBCL) in a subject in need thereof, comprising administering to a subject an IL-2 conjugate, wherein the IL-2 conjugate comprises the amino acid sequence of SEQ ID NO: 1 having an unnatural amino acid residue described herein at position 64, e.g., the amino acid sequence of SEQ ID NO: 2.

Exemplary embodiments include the following.

Embodiment 1 is a method of treating a cancer in a subject in need thereof, comprising administering to the subject (a) an IL-2 conjugate, and (b) a chimeric antigen receptor (CAR) therapy, wherein:

the IL-2 conjugate comprises the amino acid sequence of SEQ ID NO: 1 wherein the amino acid at position P64 is replaced by the structure of Formula (T).

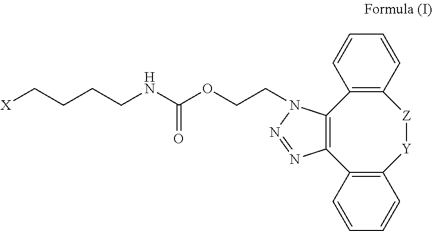

wherein:
Z is CH₂ and Y is

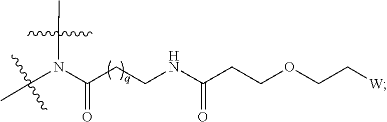

Y is CH₂ and Z is

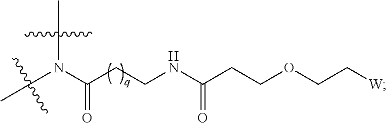

Z is CH₂ and Y is or

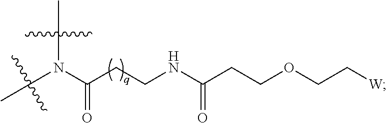

Y is CH₂ and Z is

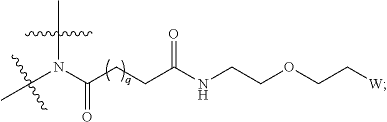

W is a PEG group having an average molecular weight of about 25 kDa-35 kDa;
q is 1, 2, or 3;
X is an L-amino acid having the structure:

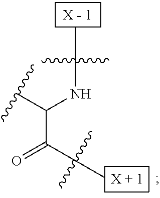

X−1 indicates the point of attachment to the preceding amino acid residue; and
X+1 indicates the point of attachment to the following amino acid residue.

Embodiment 2 is the method of embodiment 1, wherein the cancer is leukemia, myeloma, or lymphoma.

Embodiment 3 is the method of embodiment 2, wherein the leukemia is acute lymphoblastic leukemia or chronic lymphocytic leukemia.

Embodiment 4 is the method of embodiment 2, wherein the myeloma is multiple myeloma.

Embodiment 5 is the method of embodiment 2, wherein the lymphoma is non-Hodgkin lymphoma, follicular lymphoma, transformed follicular lymphoma, mantle cell lymphoma, primary mediastinal B-cell lymphoma, Burkitt's lymphoma, or diffuse large B-cell lymphoma (DLBCL).

Embodiment 6 is the method of embodiment 2 or embodiment 5, wherein the lymphoma is Burkitt's lymphoma.

Embodiment 7 is the method of embodiment 2 or embodiment 5, wherein the lymphoma is diffuse large B-cell lymphoma (DLBCL).

Embodiment 8 is a method of treating diffuse large B-cell lymphoma (DLBCL) in a subject in need thereof, comprising administering to the subject an IL-2 conjugate, wherein: the IL-2 conjugate comprises the amino acid sequence of SEQ ID NO: 1 wherein the amino acid at position P64 is replaced by the structure of Formula (I):

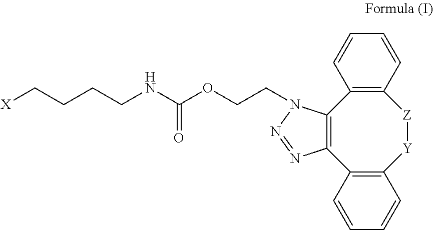

wherein:
Z is CH₂ and Y is

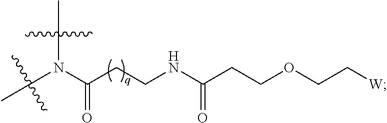

Y is CH₂ and Z is

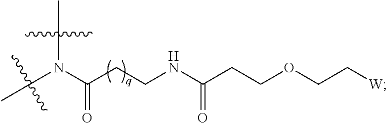

Z is CH₂ and Y is or

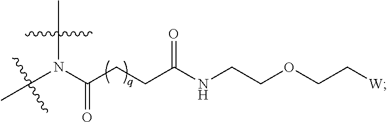

Y is CH₂ and Z is

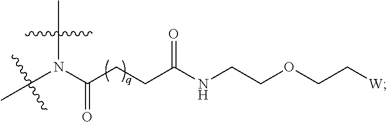

W is a PEG group having an average molecular weight of about 25 kDa-35 kDa;
q is 1, 2, or 3;
X is an L-amino acid having the structure:

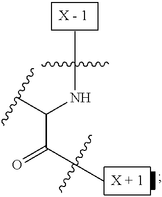

X−1 indicates the point of attachment to the preceding amino acid residue; and
X+1 indicates the point of attachment to the following amino acid residue.

Embodiment 9 is the method of embodiment 7 or embodiment 8, wherein the DLBCL is relapsed or refractory DLBCL, or the DLBCL has relapsed after two or more prior lines of systemic therapy for DLBCL.

Embodiment 10 is the method of any one of embodiments 7-9, further comprising selecting the subject to whom the IL-2 conjugate is administered at least in part on the basis of the subject having received two or more prior lines of systemic therapy for DLBCL.

Embodiment 11 is the method of embodiment 9 or embodiment 10, wherein the two or more prior lines of systemic therapy for DLBCL comprises an anthracycline, an anti-CD20 agent, or a combination of an anthracycline and an anti-CD20 agent.

Embodiment 12 is the method of embodiment 11, wherein the anti-CD20 agent comprises rituximab.

Embodiment 13 is the method of any one of embodiments 9-12, wherein the two or more prior lines of systemic therapy for DLBCL comprises a CAR therapy.

Embodiment 14 is the method of embodiment 13, wherein the CAR therapy is the last line of the two or more prior lines of systemic therapy for DLBCL.

Embodiment 15 is the method of any one of embodiments 8-14, further comprising selecting the subject to whom the IL-2 conjugate is administered at least in part on the basis of the subject having received a CAR therapy.

Embodiment 16 is the method of any one of embodiments 8-15, further comprising administering to the subject a CAR therapy.

Embodiment 17 is the method of any one of embodiments 1-7 and 13-16, wherein the CAR therapy comprises T cells expressing a CAR.

Embodiment 18 is the method of any one of embodiments 1-7 and 13-17, wherein the CAR therapy comprises gamma delta T cells expressing a CAR.

Embodiment 19 is the method of any one of embodiments 1-7 and 13-17, wherein the CAR therapy comprises natural killer (NK) T cells expressing a CAR.

Embodiment 20 is the method of any one of embodiments 1-7 and 13-16, wherein the CAR therapy comprises natural killer (NK) cells expressing a CAR.

Embodiment 21 is the method of any one of embodiments 17-20, wherein the CAR comprises an anti-CD19 domain comprising an anti-CD19 heavy chain variable domain (VH) and an anti-CD19 light chain variable domain (VL).

Embodiment 22 is the method of embodiment 21, wherein the anti-CD19 VH comprises a CDR-H1 comprising the amino acid sequence of SEQ ID NO: 3, a CDR-H2 comprising the amino acid sequence of SEQ ID NO: 4, and a CDR-H3 comprising the amino acid sequence of SEQ ID NO: 5.

Embodiment 23 is the method of embodiment 21 or embodiment 22, wherein the anti-CD19 VH comprises an amino acid sequence at least 80%, 85%, 90%, 95%, 96%, 97%, 98%, or 99% identical to the amino acid sequence of SEQ ID NO: 6.

Embodiment 24 is the method of any one of embodiments 21-23, wherein the anti-CD19 VH comprises the amino acid sequence of SEQ ID NO: 6.

Embodiment 25 is the method of any one of embodiments 21-24, wherein the anti-CD19 VL comprises a CDR-L1 comprising the amino acid sequence of SEQ ID NO: 7, a CDR-L2 comprising the amino acid sequence of SEQ ID NO: 8, and a CDR-L3 comprising the amino acid sequence of SEQ ID NO: 9.

Embodiment 26 is the method of any one of embodiments 21-25, wherein the anti-CD19 VL comprises an amino acid sequence at least 80%, 85%, 90%, 95%, 96%, 97%, 98%, or 99% identical to the amino acid sequence of SEQ ID NO: 10.

Embodiment 27 is the method of any one of embodiments 21-26, wherein the anti-CD19 VL comprises the amino acid sequence of SEQ ID NO: 10.

Embodiment 28 is the method of embodiment 21, wherein the anti-CD19 VH comprises a CDR-H1 comprising the amino acid sequence of SEQ ID NO: 11, a CDR-H2 comprising the amino acid sequence of SEQ ID NO: 12, and a CDR-H3 comprising the amino acid sequence of SEQ ID NO: 13.

Embodiment 29 is the method of embodiment 21 or embodiment 28, wherein the anti-CD19 VH comprises an amino acid sequence at least 80%, 85%, 90%, 95%, 96%, 97%, 98%, or 99% identical to the amino acid sequence of SEQ ID NO: 14.

Embodiment 30 is the method of any one of embodiments 21, 28, and 29, wherein the anti-CD19 VH comprises the amino acid sequence of SEQ ID NO: 14.

Embodiment 31 is the method of any one of embodiments 21 and 28-30, wherein the anti-CD19 VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17.

Embodiment 32 is the method of any one of embodiments 21 and 28-31, wherein the anti-CD19 VL comprises an amino acid sequence at least 80%, 85%, 90%, 95%, 96%, 97%, 98%, or 99% identical to the amino acid sequence of SEQ ID NO: 18.

Embodiment 33 is the method of any one of embodiments 21 and 28-32, wherein the anti-CD19 VL comprises the amino acid sequence of SEQ ID NO: 18.

Embodiment 34 is the method of embodiment 21, wherein the anti-CD19 VH comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21.

Embodiment 35 is the method of embodiment 21 or embodiment 34, wherein the anti-CD19 VH comprises an amino acid sequence at least 80%, 85%, 90%, 95%, 96%, 97%, 98%, or 99% identical to the amino acid sequence of SEQ ID NO: 22.

Embodiment 36 is the method of any one of embodiments 21, 34, and 35, wherein the anti-CD19 VH comprises the amino acid sequence of SEQ ID NO: 22.

Embodiment 37 is the method of any one of embodiments 21 and 34-36, wherein the anti-CD19 VL comprises a CDR-L1 comprising the amino acid sequence of SEQ ID NO: 23, a CDR-L2 comprising the amino acid sequence of SEQ ID NO: 24, and a CDR-L3 comprising the amino acid sequence of SEQ ID NO: 25.

Embodiment 38 is the method of any one of embodiments 21 and 34-37, wherein the anti-CD19 VL comprises an amino acid sequence at least 80%, 85%, 90%, 95%, 96%, 97%, 98%, or 99% identical to the amino acid sequence of SEQ ID NO: 26.

Embodiment 39 is the method of any one of embodiments 21 and 34-38, wherein the anti-CD19 VL comprises the amino acid sequence of SEQ ID NO: 26.

Embodiment 40 is the method of any one of embodiments 17-39, wherein the CAR comprises an anti-CD22 domain comprising an anti-CD22 heavy chain variable domain (VH) and an anti-CD22 light chain variable domain (VL).

Embodiment 41 is the method of embodiment 40, wherein the anti-CD22 VH comprises a CDR-H1 comprising the amino acid sequence of SEQ ID NO: 27, a CDR-H2 comprising the amino acid sequence of SEQ ID NO: 28, and a CDR-H3 comprising the amino acid sequence of SEQ ID NO: 29.

Embodiment 42 is the method of embodiment 40 or embodiment 41, wherein the anti-CD22 VH comprises an amino acid sequence at least 80%, 85%, 90%, 95%, 96%, 97%, 98%, or 99% identical to the amino acid sequence of SEQ ID NO: 30.

Embodiment 43 is the method of any one of embodiments 40-42, wherein the anti-CD22 VH comprises the amino acid sequence of SEQ ID NO: 30.

Embodiment 44 is the method of any one of embodiments 40-43, wherein the anti-CD22 VL comprises a CDR-L1 comprising the amino acid sequence of SEQ ID NO: 31, a CDR-L2 comprising the amino acid sequence of SEQ ID NO: 32, and a CDR-L3 comprising the amino acid sequence of SEQ ID NO: 33.

Embodiment 45 is the method of any one of 40-44, wherein the anti-CD22 VL comprises an amino acid sequence at least 80%, 85%, 90%, 95%, 96%, 97%, 98%, or 99% identical to the amino acid sequence of SEQ ID NO: 34.

Embodiment 46 is the method of any one of embodiments 40-45, wherein the anti-CD22 VL comprises the amino acid sequence of SEQ ID NO: 34.

Embodiment 47 is the method of any one of embodiments 17-39, wherein the CAR is an anti-CD19 CAR.

Embodiment 48 is the method of embodiment 47, wherein the anti-CD19 CAR comprises an amino acid sequence at least 80%, 85%, 90%, 95%, 96%, 97%, 98%, or 99% identical to the amino acid sequence of SEQ ID NO: 45.

Embodiment 49 is the method of embodiment 47 or embodiment 48, wherein the anti-CD19 CAR comprises the amino acid sequence of SEQ ID NO: 45.

Embodiment 50 is the method of embodiment 47, wherein the anti-CD19 CAR comprises an amino acid sequence at least 80%, 85%, 90%, 95%, 96%, 97%, 98%, or 99% identical to the amino acid sequence of SEQ ID NO: 47.

Embodiment 51 is the method of embodiment 47 or embodiment 50, wherein the anti-CD19 CAR comprises the amino acid sequence of SEQ ID NO: 47.

Embodiment 52 is the method of any one of embodiments 17-46, wherein the CAR is an anti-CD19/CD22 CAR.

Embodiment 53 is the method of embodiment 52, wherein the anti-CD19/CD22 CAR comprises an amino acid sequence at least 80%, 85%, 90%, 95%, 96%, 97%, 98%, or 99% identical to the amino acid sequence of SEQ ID NO: 43.

Embodiment 54 is the method of embodiment 52 or embodiment 53, wherein the anti-CD19/CD22 CAR comprises the amino acid sequence of SEQ ID NO: 43.

Embodiment 55 is the method of any one of embodiments 17-20, wherein the CAR is an anti-BCMA CAR.

Embodiment 56 is the method of embodiment 55, wherein the anti-BCMA CAR comprises an amino acid sequence at least 80%, 85%, 90%, 95%, 96%, 97%, 98%, or 99% identical to the amino acid sequence of SEQ ID NO: 57.

Embodiment 57 is the method of embodiment 55 or embodiment 56, wherein the anti-BCMA CAR comprises the amino acid sequence of SEQ ID NO: 57.

Embodiment 58 is the method of embodiment 55, wherein the anti-BCMA CAR comprises an amino acid sequence at least 80%, 85%, 90%, 95%, 96%, 97%, 98%, or 99% identical to the amino acid sequence of SEQ ID NO: 67.

Embodiment 59 is the method of embodiment 55 or embodiment 56, wherein the anti-BCMA CAR comprises the amino acid sequence of SEQ ID NO: 67.

Embodiment 60 is the method of any one of embodiments 17-20, wherein the CAR comprises an anti-BCMA domain comprising an anti-BCMA heavy chain variable domain (VH) and an anti-BCMA light chain variable domain (VL).

Embodiment 61 is the method of embodiment 60, wherein the anti-BCMA VH comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 51.

Embodiment 62 is the method of embodiment 60 or embodiment 61, wherein the anti-BCMA VH comprises an amino acid sequence at least 80%, 85%, 90%, 95%, 96%, 97%, 98%, or 99% identical to the amino acid sequence of SEQ ID NO: 52.

Embodiment 63 is the method of any one of embodiments 60-62, wherein the anti-BCMA VH comprises the amino acid sequence of SEQ ID NO: 52.

Embodiment 64 is the method of any one of embodiments 60-63, wherein the anti-BCMA VL comprises a CDR-L1 comprising the amino acid sequence of SEQ ID NO: 53, a CDR-L2 comprising the amino acid sequence of SEQ ID NO: 54, and a CDR-L3 comprising the amino acid sequence of SEQ ID NO: 55.

Embodiment 65 is the method of any one of embodiments 60-64, wherein the anti-BCMA VL comprises an amino acid sequence at least 80%, 85%, 90%, 95%, 96%, 97%, 98%, or 99% identical to the amino acid sequence of SEQ ID NO: 56.

Embodiment 66 is the method of any one of embodiments 60-65, wherein the anti-BCMA VL comprises the amino acid sequence of SEQ ID NO: 56.

Embodiment 67 is the method of any one of embodiments 17-20, wherein the CAR comprises an anti-BCMA domain comprising a first anti-BCMA single domain antibody (V_{H}H) and/or a second anti-BCMA V_{H}H.

Embodiment 68 is the method of embodiment 67, wherein the first anti-BCMA V_{H}H comprises a CDR-H1 comprising the amino acid sequence of SEQ ID NO: 59, a CDR-H2 comprising the amino acid sequence of SEQ ID NO: 60, and a CDR-H3 comprising the amino acid sequence of SEQ ID NO: 61.

Embodiment 69 is the method of embodiment 67 or embodiment 68, wherein the first anti-BCMA V_{H}H comprises an amino acid sequence at least 80%, 85%, 90%, 95%, 96%, 97%, 98%, or 99% identical to the amino acid sequence of SEQ ID NO: 62.

Embodiment 70 is the method of any one of embodiments 67-69, wherein the first anti-BCMA V_{H}H comprises the amino acid sequence of SEQ ID NO: 62.

Embodiment 71 is the method of any one of embodiments 67-70, wherein the second anti-BCMA V_{H}H comprises a CDR-H1 comprising the amino acid sequence of SEQ ID NO: 63, a CDR-H2 comprising the amino acid sequence of SEQ ID NO: 64, and a CDR-H3 comprising the amino acid sequence of SEQ ID NO: 65.

Embodiment 72 is the method of any one of embodiments 67-71, wherein the second anti-BCMA V_{H}H comprises an amino acid sequence at least 80%, 85%, 90%, 95%, 96%, 97%, 98%, or 99% identical to the amino acid sequence of SEQ ID NO: 66.

Embodiment 73 is the method of any one of embodiments 67-72, wherein the second anti-BCMA V_{H}H comprises the amino acid sequence of SEQ ID NO: 66.

Embodiment 74 is the method of any one of embodiments 13-73, wherein the CAR therapy comprises 0.2-5.0×10⁶ or 0.5-1.0×10⁶ of the cells per kg body weight of the subject.

Embodiment 75 is the method of any one of embodiments 13-74, wherein the CAR therapy comprises 1.0×10⁶ or 2.0×10⁶ of the cells per kg body weight of the subject.

Embodiment 76 is the method of any one of embodiments 13-75, wherein the CAR therapy comprises 0.1-2.5×10⁸ or 0.6-6.0×10⁸ of the cells.

Embodiment 77 is the method of any one of embodiments 13-76, wherein the CAR therapy comprises 0.9-1.1×10⁸, 0.5-1.1×10⁸, or 3.0-4.6×10⁸ of the cells.

Embodiment 78 is the method of any one of embodiments 16-77, wherein the CAR therapy is administered to the subject by intravenous administration.

Embodiment 79 is the method of any one of embodiments 1-7 and 16-78, wherein the CAR therapy is administered to the subject before any dose the IL-2 conjugate is administered to the subject.

Embodiment 80 is the method of any one of embodiments 1-7 and 16-78, wherein one dose of the IL-2 conjugate is administered to the subject before the CAR therapy is administered to the subject, and wherein one or more additional doses of the IL-2 conjugate is administered to the subject after the CAR therapy is administered to the subject.

Embodiment 81 is the method of any one of embodiments 1-7 and 16-78, wherein one dose of the IL-2 conjugate is administered to the subject one day before the CAR therapy is administered to the subject, and wherein one or more additional doses of the IL-2 conjugate is administered to the subject about once every week after the one dose of the IL-2 conjugate is administered to the subject.

Embodiment 82 is the method of any one of embodiments 1-81, comprising administering to the subject about 16 μg/kg IL-2 as the IL-2 conjugate.

Embodiment 83 is the method of any one of embodiments 1-81, comprising administering to the subject about 24 μg/kg IL-2 as the IL-2 conjugate.

Embodiment 84 is the method of any one of embodiments 1-81, comprising administering to the subject about 32 μg/kg IL-2 as the IL-2 conjugate.

Embodiment 85 is the method of any one of embodiments 1-84, wherein in the IL-2 conjugate the PEG group has an average molecular weight of about 30 kDa.

Embodiment 86 is the method of any one of embodiments 1-85, wherein in the IL-2 conjugate Z is CH₂ and Y is

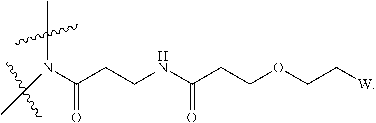

Embodiment 87 is the method of any one of embodiments 1-85, wherein in the IL-2 conjugate Y is CH₂ and Z is

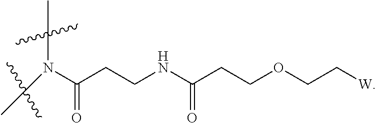

Embodiment 88 is the method of any one of embodiments 1-85, wherein in the IL-2 conjugate Z is CH₂ and Y is

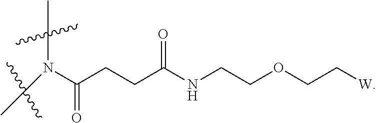

Embodiment 89 is the method of any one of embodiments 1-85, wherein in the IL-2 conjugate Y is CH₂ and Z is

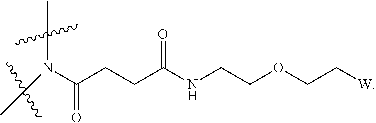

Embodiment 90 is the method of any one of embodiments 1-85, wherein the structure of Formula (I) has the structure of Formula (IV) or Formula (V), or is a mixture of Formula (IV) and Formula (V):

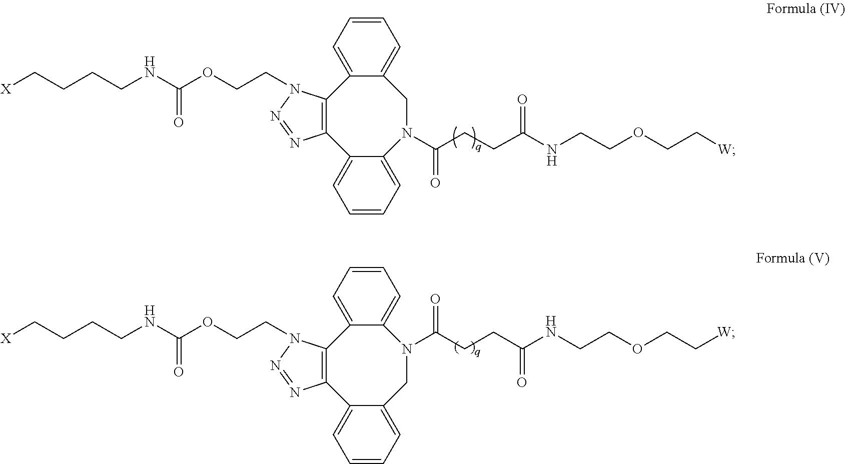

wherein:
q is 1, 2, or 3;
X is an L-amino acid having the structure:

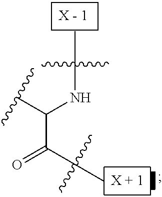

X−1 indicates the point of attachment to the preceding amino acid residue; and
X+1 indicates the point of attachment to the following amino acid residue.

Embodiment 91 is the method of any one of embodiments 1-85, wherein the structure of Formula (I) has the structure of Formula (XII) or Formula (XIII), or is a mixture of Formula (XII) and Formula (XIII):

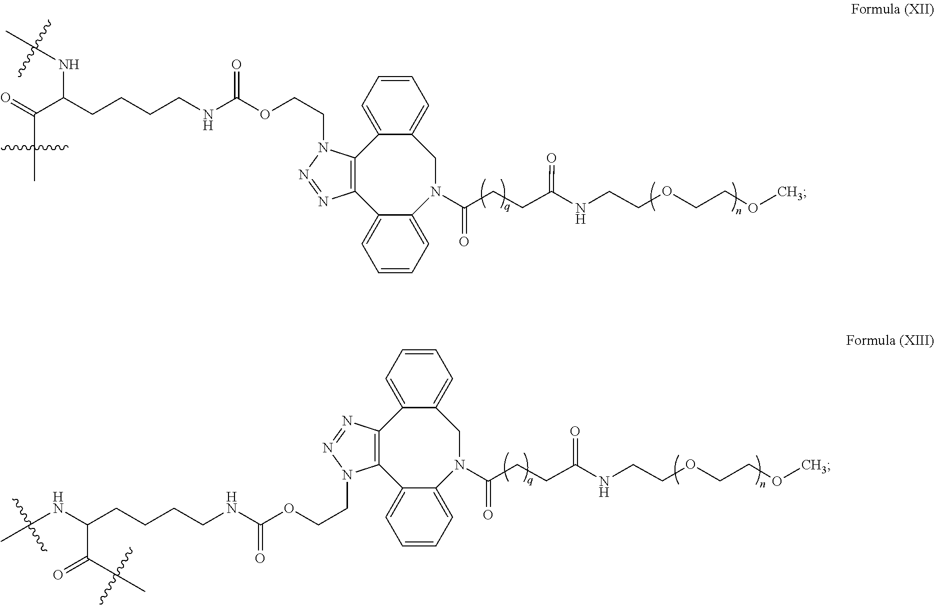

wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

Embodiment 92 is the method of any one of embodiments 1-91, wherein q is 1.

Embodiment 93 is the method of any one of embodiments 1-91, wherein q is 2.

Embodiment 94 is the method of any one of embodiments 1-91, wherein q is 3.

Embodiment 95 is the method of any one of embodiments 1-94, wherein the IL-2 conjugate is administered to the subject about once every week, about once every two weeks, about once every three weeks, or about once every 4 weeks.

Embodiment 96 is the method of embodiment 95, wherein the IL-2 conjugate is administered to the subject about once every three weeks.

Embodiment 97 is the method of any one of embodiments 1-96, wherein the IL-2 conjugate is a pharmaceutically acceptable salt, solvate, or hydrate.

Embodiment 98 is the method of any one of embodiments 1-97, wherein the IL-2 conjugate is administered to the subject by intravenous administration.

Embodiment 99 is the method of any one of embodiments 1-97, wherein the IL-2 conjugate is administered to the subject by subcutaneous administration.

Embodiment 100 is the method of any one of embodiments 1-99, further comprising administering acetaminophen to the subject.

Embodiment 101 is the method of any one of embodiments 1-100, further comprising administering diphenhydramine to the subject.

Embodiment 102 is the method of embodiment 100 or embodiment 101, wherein the acetaminophen and/or diphenhydramine is administered to the subject before administering the IL-2 conjugate.

Embodiment 103 is an IL-2 conjugate for use in the method of any one of embodiments 1-102.

Embodiment 104 is use of an IL-2 conjugate for the manufacture of a medicament for the method of any one of embodiments 1-102.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the change in peripheral CD8+ T_{eff} counts in the indicated subjects treated with 24 μg/kg [Q3W] of the IL-2 conjugate at specified times following administration of IL-2 conjugate. Here and elsewhere, designations such as “C1D1” indicate the treatment cycle and day (e.g., treatment cycle 1, day 1). “PRE” indicates the baseline measurement before administration; 24 HR indicates 24 hours after administration; and so on.

FIG. 1B shows the peak peripheral CD8+ T_{eff} cell expansion following administration of the first dose of 24 μg/kg [Q3W] of the IL-2 conjugate. Data is normalized to pre-treatment (C1D1) CD8+ T cell count.

FIG. 1C shows the peripheral CD8+ T_{eff} cell counts in the indicated subjects treated with 24 μg/kg [Q3W] of the IL-2 conjugate at specified times following administration of IL-2 conjugate.

FIG. 2 shows the percentage of CD8+ T_{eff} cells expressing Ki67 in the indicated subjects treated with 24 μg/kg [Q3W] of the IL-2 conjugate at specified times following administration of IL-2 conjugate.

FIG. 3A shows the change in CD8+ memory cell counts in the indicated subjects treated with 24 μg/kg [Q3W] of the IL-2 conjugate at specified times following administration of IL-2 conjugate.

FIG. 3B shows CD8+ memory cell counts in the indicated subjects treated with 24 μg/kg [Q3W] of the IL-2 conjugate at specified times following administration of IL-2 conjugate.

FIG. 4A shows the change in peripheral natural killer (NK) cell counts in the indicated subjects treated with 24 μg/kg [Q3W] of the IL-2 conjugate at specified times following administration of IL-2 conjugate.

FIG. 4B shows the peak peripheral NK cell expansion following administration of the first dose of 24 μg/kg [Q3W] of the IL-2 conjugate. Data is normalized to pre-treatment (C1D1) NK cell count.

FIG. 4C shows the change in peripheral natural killer (NK) cell counts in the indicated subjects treated with 24 μg/kg [Q3W] of the IL-2 conjugate at specified times following administration of IL-2 conjugate.

FIG. 4D shows peripheral natural killer (NK) cell counts in the indicated subjects treated with 24 μg/kg [Q3W] of the IL-2 conjugate at specified times following administration of IL-2 conjugate.

FIG. 5 shows the percentage of NK cells expressing Ki67 in the indicated subjects treated with 24 μg/kg [Q3W] of the IL-2 conjugate at specified times following administration of IL-2 conjugate.

FIG. 6A shows the change in peripheral CD4+ T_{reg} counts in the indicated subjects treated with 24 μg/kg [Q3W] of the IL-2 conjugate at specified times following administration of IL-2 conjugate.

FIG. 6B shows the peak peripheral CD4+ T_{reg} cell expansion following administration of the first dose of 24 μg/kg [Q3W] of the IL-2 conjugate. Data is normalized to pre-treatment (C1D1) CD4+ T cell count.

FIG. 6C shows the peripheral CD4+ T_{reg} cell counts in the indicated subjects treated with 24 μg/kg [Q3W] of the IL-2 conjugate at specified times following administration of IL-2 conjugate.

FIG. 7 shows the percentage of CD4+ T_{reg} cells expressing Ki67 in the indicated subjects treated with 24 μg/kg [Q3W] of the IL-2 conjugate at specified times following administration of IL-2 conjugate.

FIG. 8A shows the change in eosinophil cell counts in the indicated subjects treated with 24 μg/kg [Q3W] of the IL-2 conjugate at specified times following administration of IL-2 conjugate.

FIG. 8B shows the peak peripheral eosinophil cell expansion following administration of the first dose of 24 μg/kg [Q3W] of the IL-2 conjugate. Data is normalized to pre-treatment (C1D1) eosinophil cell count.

FIG. 8C shows eosinophil cell counts in the indicated subjects treated with 24 μg/kg [Q3W] of the IL-2 conjugate at specified times following administration of IL-2 conjugate.

FIG. 9A shows serum levels of IFN-γ, IL-5, and IL-6 in the indicated subjects treated with 24 μg/kg [Q3W] of the IL-2 conjugate at specified times following administration of IL-2 conjugate.

FIG. 9B shows the serum level of IL-5 following administration of 24 μg/kg [Q3W] of IL-2 conjugate. BLQ=below limit of quantification. Data is plotted as mean (range BLQ to maximum value).

FIG. 9C shows the serum level of IL-6 following administration of 24 μg/kg [Q3W] of IL-2 conjugate. BLQ=below limit of quantification. Data is plotted as mean (range BLQ to maximum value).

FIG. 10 shows serum levels of the indicated cytokines in the indicated subjects treated with 8 μg/kg [Q3W] at specified times following IL-2 conjugate administration.

FIG. 11 shows serum levels of the indicated cytokines in the indicated subjects treated with 16 μg/kg [Q3W] at specified times following IL-2 conjugate administration.

FIGS. 12A-12D show eosinophil cell counts in the indicated subjects treated with 8 μg/kg [Q3W] or 16 μg/kg [Q3W] at specified times following IL-2 conjugate administration as measured by cytometry or CBC (complete blood count).

FIGS. 13A-13D show lymphocyte counts in the indicated subjects treated with 8 μg/kg [Q3W] or 16 μg/kg [Q3W] at specified times following IL-2 conjugate administration as measured by cytometry or CBC.

FIGS. 14A-14D show peripheral CD8+ T_{eff} counts in the indicated subjects treated with 8 μg/kg [Q3W] or 16 μg/kg [Q3W] at specified times following IL-2 conjugate administration.

FIGS. 15A-15B show the percentage of CD8+ T_{eff} cells expressing Ki67 in the indicated subjects treated with 8 μg/kg [Q3W] or 16 μg/kg [Q3W] at specified times following IL-2 conjugate administration.

FIGS. 16A-16B show peripheral memory CD8+ counts in the indicated subjects treated with 8 μg/kg [Q3W] or 16 μg/kg [Q3W] at specified times following IL-2 conjugate administration.

FIGS. 17A-17D show peripheral natural killer (NK) cell counts in the indicated subjects treated with 8 μg/kg [Q3W] or 16 μg/kg [Q3W] at specified times following IL-2 conjugate administration.

FIGS. 18A-18B show the percentage of NK cells expressing Ki67 in the indicated subjects treated with 8 μg/kg [Q3W] or 16 μg/kg [Q3W] at specified times following IL-2 conjugate administration.

FIGS. 19A-19B show peripheral CD4+ T_{reg} counts in the indicated subjects treated with 8 μg/kg [Q3W] or 16 μg/kg [Q3W] at specified times following IL-2 conjugate administration.

FIGS. 20A-20B show the percentage of CD4+ T_{reg} cells expressing Ki67 in the indicated subjects treated with 8 μg/kg [Q3W] or 16 μg/kg [Q3W] at specified times following IL-2 conjugate administration.

FIG. 21A shows the change in peripheral CD8+ T_{eff} counts in the indicated subjects treated with 32 μg/kg [Q3W] of the IL-2 conjugate at specified times following administration of IL-2 conjugate.

FIG. 21B shows the peripheral CD8+ T_{eff} cell counts in the indicated subjects treated with 32 μg/kg [Q3W] of the IL-2 conjugate at specified times following administration of IL-2 conjugate.

FIG. 22A shows the change in CD8+ memory cell counts in the indicated subjects treated with 32 μg/kg [Q3W] of the IL-2 conjugate at specified times following administration of IL-2 conjugate.

FIG. 22B shows CD8+ memory cell counts in the indicated subjects treated with 32 μg/kg [Q3W] of the IL-2 conjugate at specified times following administration of IL-2 conjugate.

FIG. 23A shows the change in peripheral natural killer (NK) cell counts in the indicated subjects treated with 32 μg/kg [Q3W] of the IL-2 conjugate at specified times following administration of IL-2 conjugate.

FIG. 23B shows peripheral natural killer (NK) cell counts in the indicated subjects treated with 32 μg/kg [Q3W] of the IL-2 conjugate at specified times following administration of IL-2 conjugate.

FIG. 24A shows the change in peripheral CD4+ T_{reg} counts in the indicated subjects treated with 32 μg/kg [Q3W] of the IL-2 conjugate at specified times following administration of IL-2 conjugate.

FIG. 24B shows the peripheral CD4+ T_{reg} cell counts in the indicated subjects treated with 32 μg/kg [Q3W] of the IL-2 conjugate at specified times following administration of IL-2 conjugate.

FIG. 25A shows the change in eosinophil cell counts in the indicated subjects treated with 32 μg/kg [Q3W] of the IL-2 conjugate at specified times following administration of IL-2 conjugate.

FIG. 25B shows eosinophil cell counts in the indicated subjects treated with 32 μg/kg [Q3W] of the IL-2 conjugate at specified times following administration of IL-2 conjugate.

FIG. 26 shows serum levels of IFN-γ, IL-5, and IL-6 in the indicated subjects treated with 24 μg/kg [Q3W] of the IL-2 conjugate at specified times following administration of IL-2 conjugate.

FIG. 27A shows cell counts in a co-culture of CAR-T cells with large B cell lymphoma cells. FIG. 27B shows flow cytometry data of cells in the co-culture, indicating an increase in the proportion of effector memory (EM) cells.

FIG. 28 shows average radiance in mice engrafted with Raji-Luc cells and treated with CAR-T cells and an IL-2 conjugate. FIG. 29 shows imaging of the radiance in the mice.

FIG. 30 shows body weight changes of the mice. FIG. 31 shows copy numbers of the CAR-T construct in the mice, as determined by droplet digital polymerase chain reaction (ddPCR).

FIG. 32A shows average radiance in mice engrafted with Raji-Luc cells and treated with CAR-T cells and an IL-2 conjugate. FIG. 32B shows body weight changes of the mice. FIG. 33 shows the quantities of CD4+ CAR-T cells and CD8+ CAR-T cells in the mice. FIG. 34 shows the the quantities of different types of cells in the mice. FIG. 35 shows copy numbers of the CAR-T construct, as determined by ddPCR.

FIG. 36 shows CAR expression in NK cells from two donors.

FIG. 37 shows Nalm6 cancer cell killing by CAR-NK cells in the presence of an IL-2 conjugate.

FIG. 38 shows IFN-γ production from co-cultures of CAR-NK cells and Nalm6 cells in the presence of an IL-2 conjugate.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “subject(s)” and “patient(s)” mean any mammal. In some embodiments, the mammal is a human. In some embodiments, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285 (15): 11039-11044 (2010), the disclosure of which is incorporated herein by reference.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; single-chain antibody molecules (e.g., scFv); and multispecific antibodies formed from antibody fragments.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “dose-limiting toxicity” (DLT) is defined as an adverse event occurring within a pre-determined time window (e.g., within Day 1 through Day 29 (inclusive)+1 day) of a treatment cycle that was not clearly or incontrovertibly solely related to an extraneous cause and that meets one or more of the criteria set forth in Example 2 for DLT.

As used herein, “severe cytokine release syndrome” refers to level 4 or 5 cytokine release syndrome as described in Teachey et al., Cancer Discov. 2016; 6 (6); 664-79, the disclosure of which is incorporated herein by reference.

As used herein, “chimeric antigen receptor” or “CAR” describes a fusion protein comprising an extracellular antigen-binding component, which may be a single chain variable fragment (scFv or sFv) derived from a VH and VL (e.g., of a monoclonal antibody, Fab, or the like), that is fused to a transmembrane domain and an intracellular signaling domain capable of activating or stimulating an immune cell.

As used herein, the assignment of amino acids in complementarity-determining regions (CDRs) to a heavy chain variable domain (VH) or a light chain variable domain (VL) is in accordance with the definitions of Kabat et al. in Sequences of Proteins of Immunological Interest, 5th Ed., US Dept. of Health and Human Services, PHS, NIH, NIH Publication no. 91-3242, 1991; IMGT® (international ImMunoGeneTics information system; Lefranc et al, Dev. Comp. Immunol. 29:185-203; 2005); AHo (Honegger and Pluckthun, J. Mol. Biol. 309 (3): 657-670; 2001); Chothia (Al-Lazikani et al., 1997 Journal of Molecular Biology 273:927-948; or Contact (Maccallum et al., 1996 Journal of Molecular Biology 262:732-745.

The “percent identity” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2 Conjugates

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Ra (also known as CD25), IL-2Rβ (also known as CD122), and IL-2Rγ (also known as CD132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4⁺ T cells, CD8⁺ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4⁺CD25⁺T cells suppressed the development of autoimmunity (Sakaguchi, et al., “Immunologic self-tolerance maintained by activated T cells expressing IL-2 receptor alpha-chains (CD25),” J. Immunol. 155 (3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8+ effector T (Teff) cells, NK cells, and NKT cells. CD8+ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8+ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome (VLS).

Provided herein are methods of treating diffuse large B-cell lymphoma (DLBCL) in a subject in need thereof, comprising administering to the subject an IL-2 conjugate.

In some embodiments, the IL-2 sequence comprises the sequence of SEQ ID NO: 1:

(SEQ ID NO: 1)
PTSSSTKKTQLQLEHLLLDLQMILNGINNYKNPKLTRMLTFKFYMPKKAT
ELKHLQCLEEELKPLEEVLNLAQSKNFHLRPRDLISNINVIVLELKGSET
TFMCEYADETATIVEFLNRWITFSQSIISTLT

wherein the amino acid at position P64 is replaced by the structure of Formula (I):

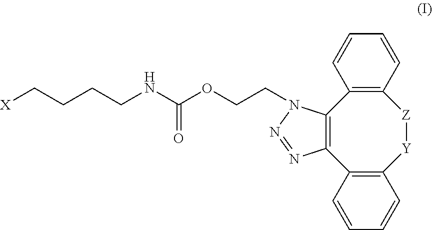

wherein:
Z is CH₂ and Y is

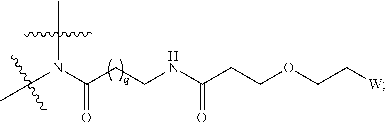

Y is CH₂ and Z is

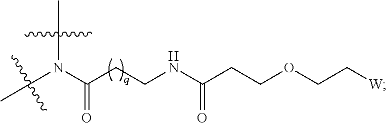

Z is CH₂ and Y is or

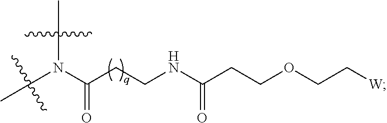

Y is CH₂ and Z is

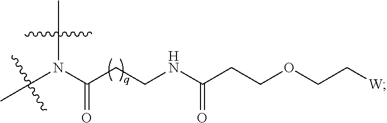

W is a PEG group having an average molecular weight of about 25 kDa-35 kDa;
q is 1, 2, or 3;
X is an L-amino acid having the structure:

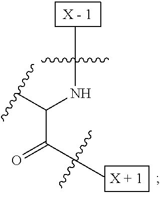

X−1 indicates the point of attachment to the preceding amino acid residue; and
X+1 indicates the point of attachment to the following amino acid residue.

In any of the embodiments or variations of Formula (I) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In some embodiments of Formula (I), Z is CH₂ and Y is

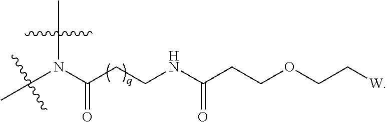

In some embodiments of Formula (I), Y is CH₂ and Z is

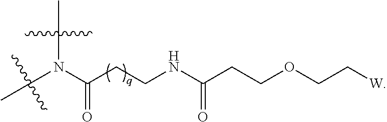

In some embodiments of Formula (I), Z is CH₂ and Y is

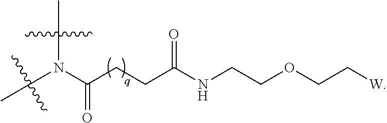

In some embodiments of Formula (I), Y is CH₂ and Z is

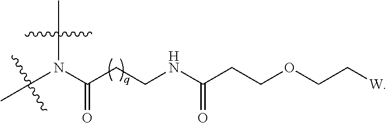

In some embodiments of Formula (I), q is 1. In some embodiments of Formula (I), q is 2. In some embodiments of Formula (I), q is 3.

In some embodiments of Formula (I), W is a PEG group having an average molecular weight of about 25 kDa. In some embodiments of Formula (I), W is a PEG group having an average molecular weight of about 30 kDa. In some embodiments of Formula (I), W is a PEG group having an average molecular weight of about 35 kDa.

In some embodiments of Formula (I), q is 1 and structure of Formula (I) is the structure of Formula (Ia):

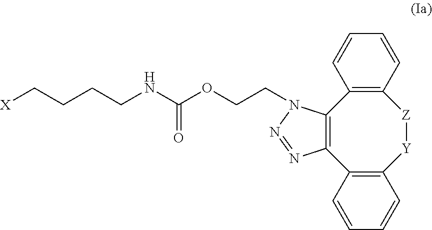

wherein:
Z is CH₂ and Y is

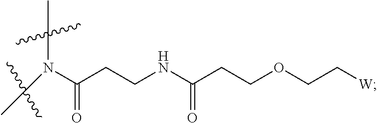

Y is CH₂ and Z is

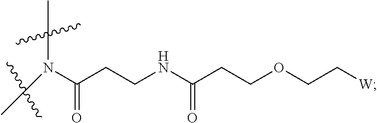

Z is CH₂ and Y is or

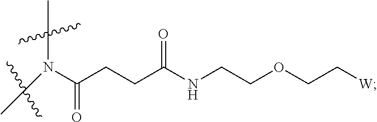

Y is CH₂ and Z is

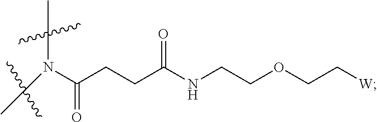

W is a PEG group having an average molecular weight of about 25 kDa-35 kDa;
X is an L-amino acid having the structure:

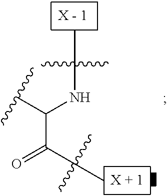

X−1 indicates the point of attachment to the preceding amino acid residue; and
X+1 indicates the point of attachment to the following amino acid residue.

In some embodiments of Formula (Ia), Z is CH₂ and Y is

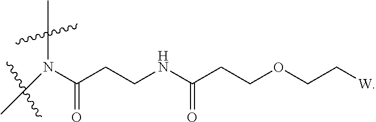

In some embodiments of Formula (Ia), Y is CH₂ and Z is

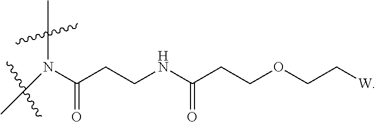

In other embodiments of Formula (Ia), Z is CH₂ and Y is

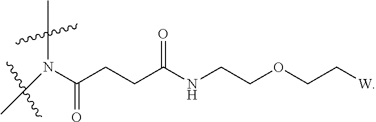

In some embodiments of Formula (Ia), Y is CH₂ and Z is

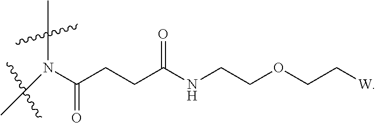

In some embodiments of Formula (Ia), the PEG group has an average molecular weight of about 30 kDa.

In some embodiments, the IL-2 conjugate comprises the sequence of SEQ ID NO: 2:

(SEQ ID NO: 2)
PTSSSTKKTQLQLEHLLLDLQMILNGINNYKNPKLTRMLTFKFYMPKKAT
ELKHLQCLEEELK[AzK L1 PEG30kD]LEEVLNLAQSKNFHLRPRDLI
SNINVIVLELKGSETTFMCEYADETATIVEFLNRWITFSQSIISTLT

wherein [AzK_L1_PEG30 kD] is N6-((2-azidoethoxy)-carbonyl)-L-lysine stably-conjugated to PEG via DBCO-mediated click chemistry to form a compound comprising a structure of Formula (IV) or Formula (V), wherein q is 1 (such as Formula (IVa) or Formula (Va)), and wherein the PEG group has an average molecular weight of about 25-35 kiloDaltons (e.g., about 30 kDa), capped with a methoxy group. The term “DBCO” means a chemical moiety comprising a dibenzocyclooctyne group, such as comprising the mPEG-DBCO compound illustrated in Schemes 1 and 2 of Example 1.

The ratio of regioisomers generated from the click reaction is about 1:1 or greater than 1:1.

PEGs typically comprise a number of (OCH₂CH₂) monomers (or (CH₂CH₂O) monomers, depending on how the PEG is defined). In some embodiments, the number of (OCH₂CH₂) monomers (or (CH₂CH₂O) monomers) is such that the average molecular weight of the PEG group is about 30 kDa.

In some instances, the PEG is an end-capped polymer, that is, a polymer having at least one terminus capped with a relatively inert group, such as a lower C₁₋₆ alkoxy group, or a hydroxyl group. In some embodiments, the PEG group is a methoxy-PEG (commonly referred to as mPEG), which is a linear form of PEG wherein one terminus of the polymer is a methoxy (—OCH₃) group, and the other terminus is a hydroxyl or other functional group that can be optionally chemically modified.

In some embodiments, the PEG group is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. For example, included within the scope of the present disclosure are IL-2 conjugates comprising a PEG group having a molecular weight of 30,000 Da±3,000 Da, or 30,000 Da±4,500 Da, or 30,000 Da±5,000 Da.

In some embodiments, the IL-2 conjugate comprises the amino acid sequence of SEQ ID NO: 1 in which the amino acid residue P64 is replaced by the structure of Formula (IV) or Formula (V), or a mixture of Formula (IV) and Formula (V):

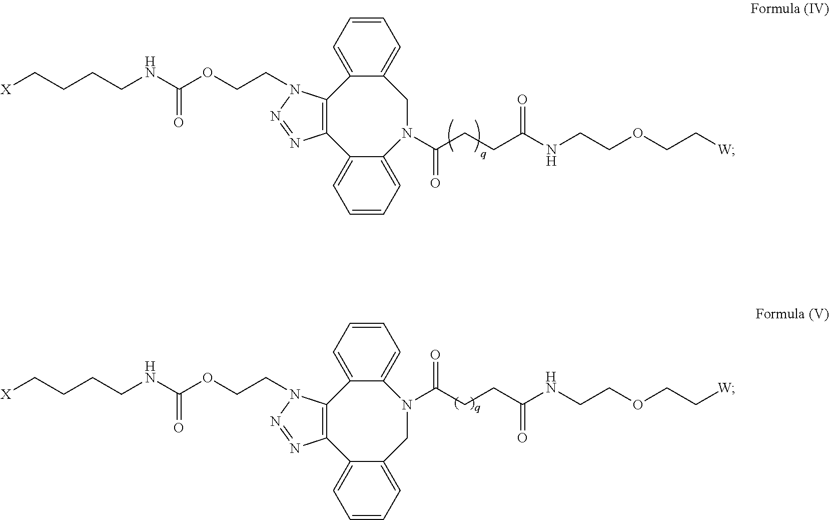

wherein:
W is a PEG group having an average molecular weight of about 25 kDa-35 kDa;
q is 1, 2, or 3; and
X has the structure:

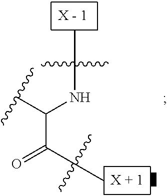

X−1 indicates the point of attachment to the preceding amino acid residue; and
X+1 indicates the point of attachment to the following amino acid residue.

In some embodiments of Formula (IV) or Formula (V), or a mixture of Formula (IV) or Formula (V), q is 1. In some embodiments of Formula (IV) or Formula (V), or a mixture of Formula (IV) or Formula (V), q is 2. In some embodiments of Formula (IV) or Formula (V), or a mixture of Formula (IV) or Formula (V), q is 3.

In some embodiments of Formula (IV) or Formula (V), or a mixture of Formula (IV) or Formula (V), W is a PEG group having an average molecular weight of about 25 kDa. In some embodiments of Formula (IV) or Formula (V), or a mixture of Formula (IV) or Formula (V), W is a PEG group having an average molecular weight of about 30 kDa. In some embodiments of Formula (IV) or Formula (V), or a mixture of Formula (IV) or Formula (V), W is a PEG group having an average molecular weight of about 35 kDa.

In any of the embodiments described herein, the structure of Formula (I) has the structure of Formula (IV) or Formula (V), or is a mixture of Formula (IV) and Formula (V). In some embodiments, the structure of Formula (I) has the structure of Formula (IV). In some embodiments, the structure of Formula (I) has the structure of Formula (V). In some embodiments, the structure of Formula (I) is a mixture of Formula (IV) and Formula (V).

In some embodiments of Formula (IV) or Formula (V), or a mixture of Formula (IV) and Formula (V), q is 1, the structure of Formula (IV) is the structure of Formula (IVa), and the structure of Formula (V) is the structure of Formula (Va):

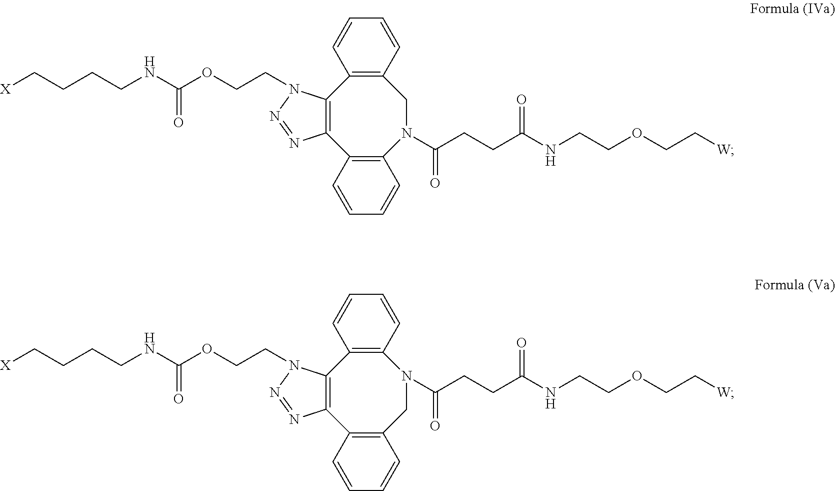

wherein:
W is a PEG group having an average molecular weight of about 25 kDa-35 kDa; and
X has the structure:

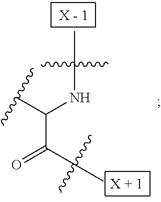 [SMILES: *NC(*)C(*)=O]

X−1 indicates the point of attachment to the preceding amino acid residue; and
X+1 indicates the point of attachment to the following amino acid residue.

In some embodiments of Formula (IVa) or Formula (Va), or a mixture of Formula (IVa) and Formula (Va), the PEG group has an average molecular weight of about 30 kDa.

In any of the embodiments described herein, the structure of Formula (I) has the structure of Formula (IVa) or Formula (Va), or is a mixture of Formula (IVa) and Formula (Va). In some embodiments, the structure of Formula (I) has the structure of Formula (IVa). In some embodiments, the structure of Formula (I) has the structure of Formula (Va). In some embodiments, the structure of Formula (I) is a mixture of Formula (IVa) and Formula (Va).

In some embodiments, the IL-2 conjugate comprises the amino acid sequence of SEQ ID NO: 1 in which the amino acid residue P64 is replaced by the structure of Formula (XII) or Formula (XIII), or a mixture of Formula (XII) and Formula (XIII):

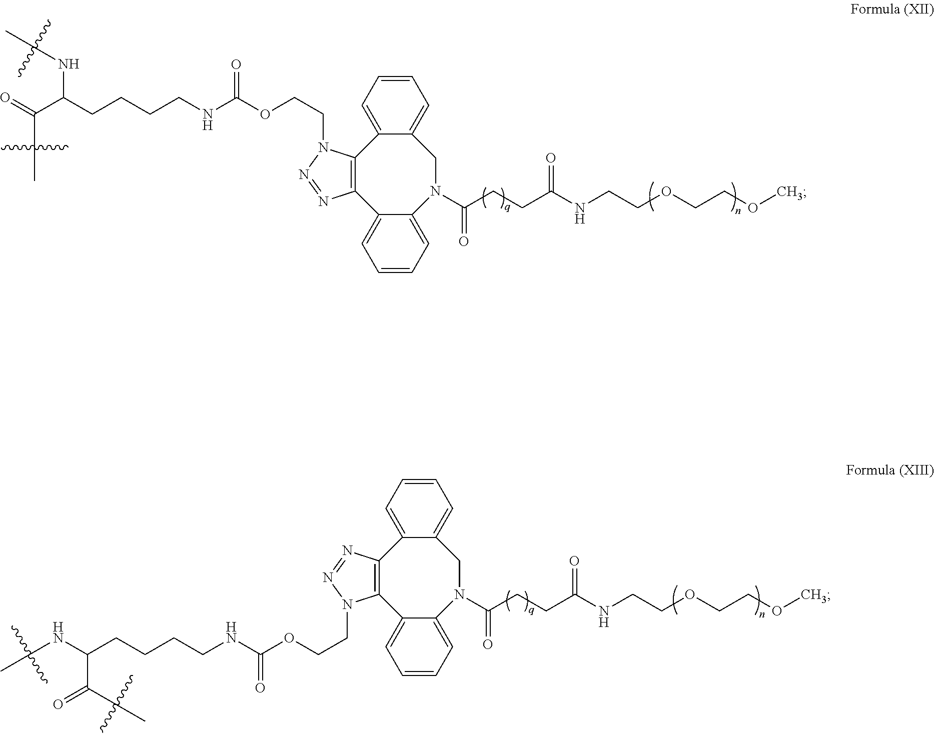

wherein:
n is is an integer such that —(OCH₂CH₂)ₙ—OCH₃ has a molecular weight of about 25 kDa-35 kDa;
q is 1, 2, or 3; and
the wavy lines indicate convalent bonds to amino acid residues within SEQ ID NO: 1 that are not replaced.

In some embodiments of Formula (XII) or Formula (XIII), or a mixture of Formula (XII) and Formula (XIII), q is 1. In some embodiments of Formula (XII) or Formula (XIII), or a mixture of Formula (XII) and Formula (XIII), q is 2. In some embodiments of Formula (XII) or Formula (XIII), or a mixture of Formula (XII) and Formula (XIII), q is 3.

In some embodiments of Formula (XII) or Formula (XIII), or a mixture of Formula (XII) and Formula (XIII), n is is an integer such that —(OCH₂CH₂)ₙ—OCH₃ has a molecular weight of about 30 kDa.

In any of the embodiments described herein, the structure of Formula (I) has the structure of Formula (XII) or Formula (XIII), or is a mixture of Formula (XII) and Formula (XIII). In some embodiments, the structure of Formula (I) has the structure of Formula (XII). In some embodiments, the structure of Formula (I) has the structure of Formula (XIII). In some embodiments, the structure of Formula (I) is a mixture of Formula (XII) and Formula (XIII).

In some embodiments of Formula (XII) or Formula (XIII), or a mixture of Formula (XII) and Formula (XIII), q is 1, the structure of Formula (XII) is the structure of Formula (XIIa), and the structure of Formula (XIII) is the structure of Formula (XIIIa):

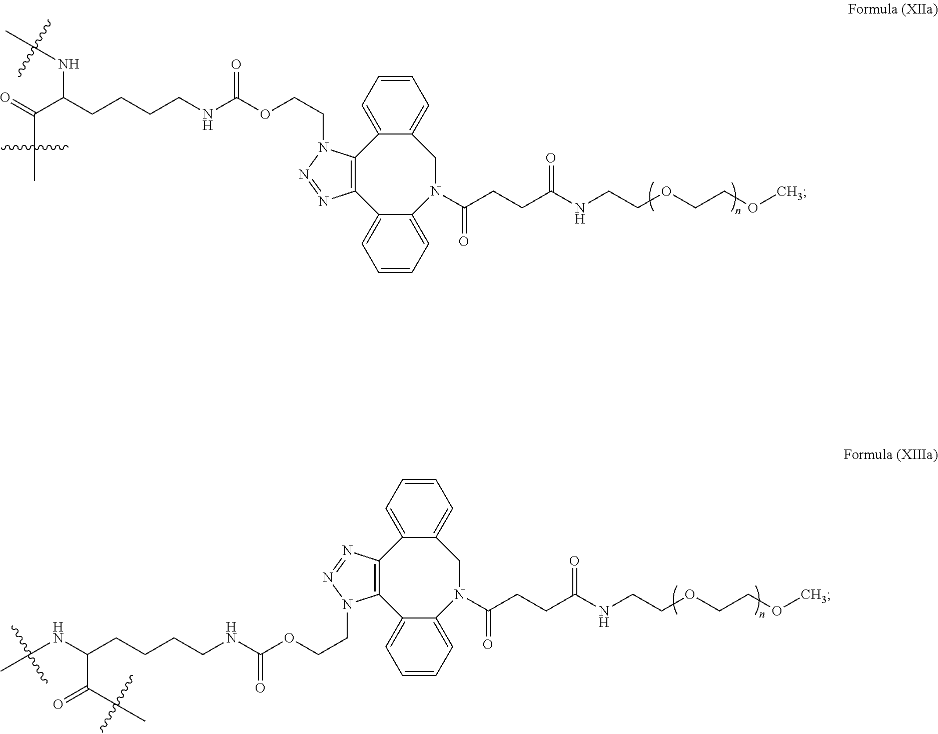

wherein:
n is is an integer such that —(OCH₂CH₂)ₙ—OCH₃ has a molecular weight of about 25 kDa-35 kDa; and
the wavy lines indicate convalent bonds to amino acid residues within SEQ ID NO: 1 that are not replaced.

In some embodiments of Formula (XIIa) or Formula (XIIIa), or a mixture of Formula (XIIa) and Formula (XIIIa), n is is an integer such that —(OCH₂CH₂)ₙ—OCH₃ has a molecular weight of about 30 kDa.

In any of the embodiments described herein, the structure of Formula (I) has the structure of Formula (XIIa) or Formula (XIIIa), or is a mixture of Formula (XIIa) and Formula (XIIIa). In some embodiments, the structure of Formula (I) has the structure of Formula (XIIa). In some embodiments, the structure of Formula (I) has the structure of Formula (XIIIa). In some embodiments, the structure of Formula (I) is a mixture of Formula (XIIa) and Formula (XIIIa).

In some embodiments, the IL-2 conjugate comprises the amino acid sequence of SEQ ID NO: 1 in which the amino acid residue P64 is replaced by the structure of Formula (XIV) or Formula (XV), or a mixture of Formula (XIV) and Formula (XV):

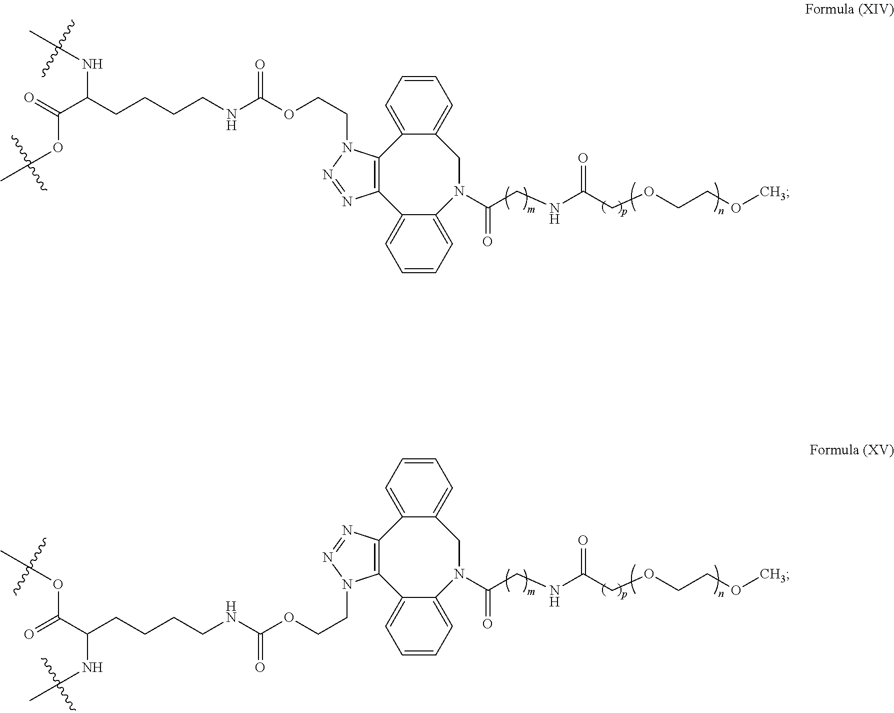

wherein:
m is an integer from 0 to 20;
p is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XIV) or Formula (XV), or a mixture of Formula (XIV) and Formula (XV), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In some embodiments, p is an integer from 0 to 15. In some embodiments, p is an integer from 0 to 10. In some embodiments, p is an integer from 0 to 5. In some embodiments, p is an integer from 1 to 5. In some embodiments, p is 1. In some embodiments, p is 2. In some embodiments, p is 3. In some embodiments, p is 4. In some embodiments, p is 5.

In some embodiments, m and p are each 2.

In any of the embodiments described herein, the structure of Formula (I) has the structure of Formula (XIV) or Formula (XV), or is a mixture of Formula (XIV) and Formula (XV). In some embodiments, the structure of Formula (I) has the structure of Formula (XIV). In some embodiments, the structure of Formula (I) has the structure of Formula (XV). In some embodiments, the structure of Formula (I) is a mixture of Formula (XIV) and Formula (XV).

In some embodiments, the IL-2 conjugate comprises the amino acid sequence of SEQ ID NO: 1 in which the amino acid residue P64 is replaced by the structure of Formula (XVI) or Formula (XVII), or a mixture of Formula (XVI) and Formula (XVII):

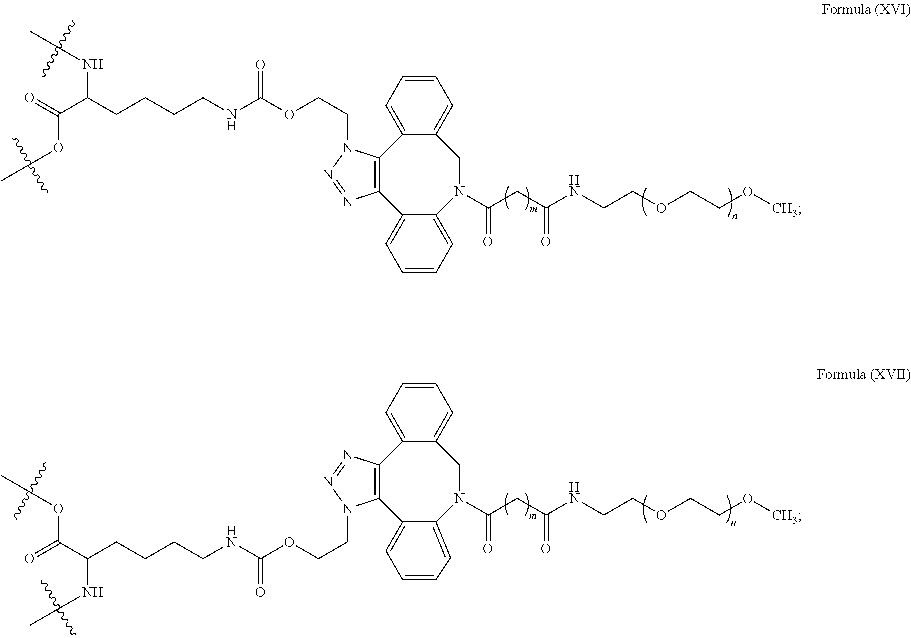

wherein:
m is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XVI) or Formula (XVII), or a mixture of Formula (XVI) and Formula (XVII), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In any of the embodiments described herein, the structure of Formula (I) has the structure of Formula (XVI) or Formula (XVII), or is a mixture of Formula (XVI) and Formula (XVII). In some embodiments, the structure of Formula (I) has the structure of Formula (XVI). In some embodiments, the structure of Formula (I) has the structure of Formula (XVII). In some embodiments, the structure of Formula (I) is a mixture of Formula (XVI) and Formula (XVII).

### Conjugation Chemistry

In some embodiments, the IL-2 conjugates described herein can be prepared by a conjugation reaction comprising a 1,3-dipolar cycloaddition reaction. In some embodiments, the 1,3-dipolar cycloaddition reaction comprises reaction of an azide and an alkyne (“Click” reaction). In some embodiments, a conjugation reaction described herein comprises the reaction outlined in Scheme I, wherein X is an unnatural amino acid at position P64 of SEQ ID NO: 1.

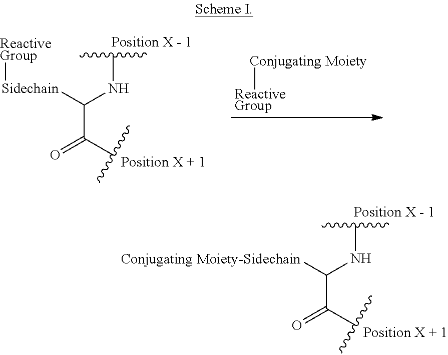

In some embodiments, the conjugating moiety comprises a PEG group as described herein. In some embodiments, a reactive group comprises an alkyne or azide.

In some embodiments, a conjugation reaction described herein comprises the reaction outlined in Scheme II, wherein X is an unnatural amino acid at position P64 of SEQ ID NO: 1.

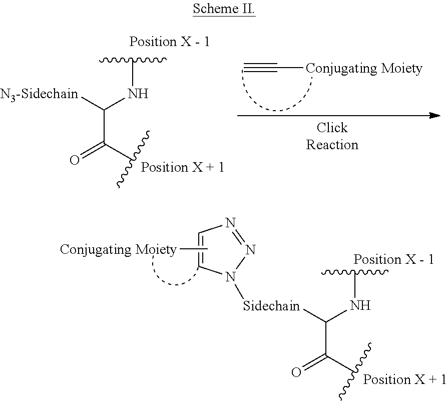

In some embodiments, a conjugation reaction described herein comprises the reaction outlined in Scheme III, wherein X is an unnatural amino acid at position P64 of SEQ ID NO: 1.

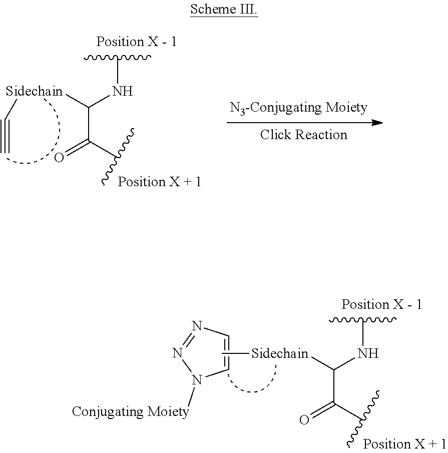

In some embodiments a conjugation reaction described herein comprises the reaction outlined in Scheme IV, wherein X is an unnatural amino acid at position P64 of SEQ ID NO: 1.

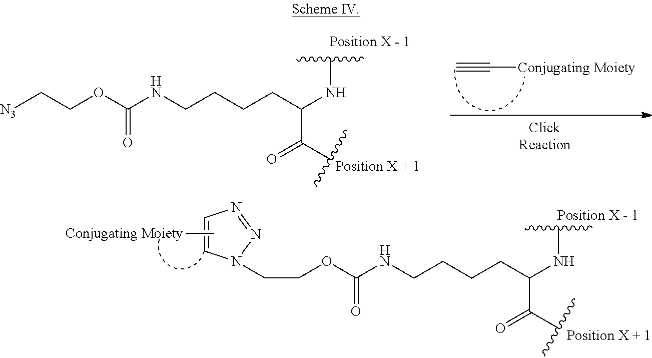

In some embodiments, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods known to those of ordinary skill in the art. Exemplary reactions are shown in Schemes V and VI.

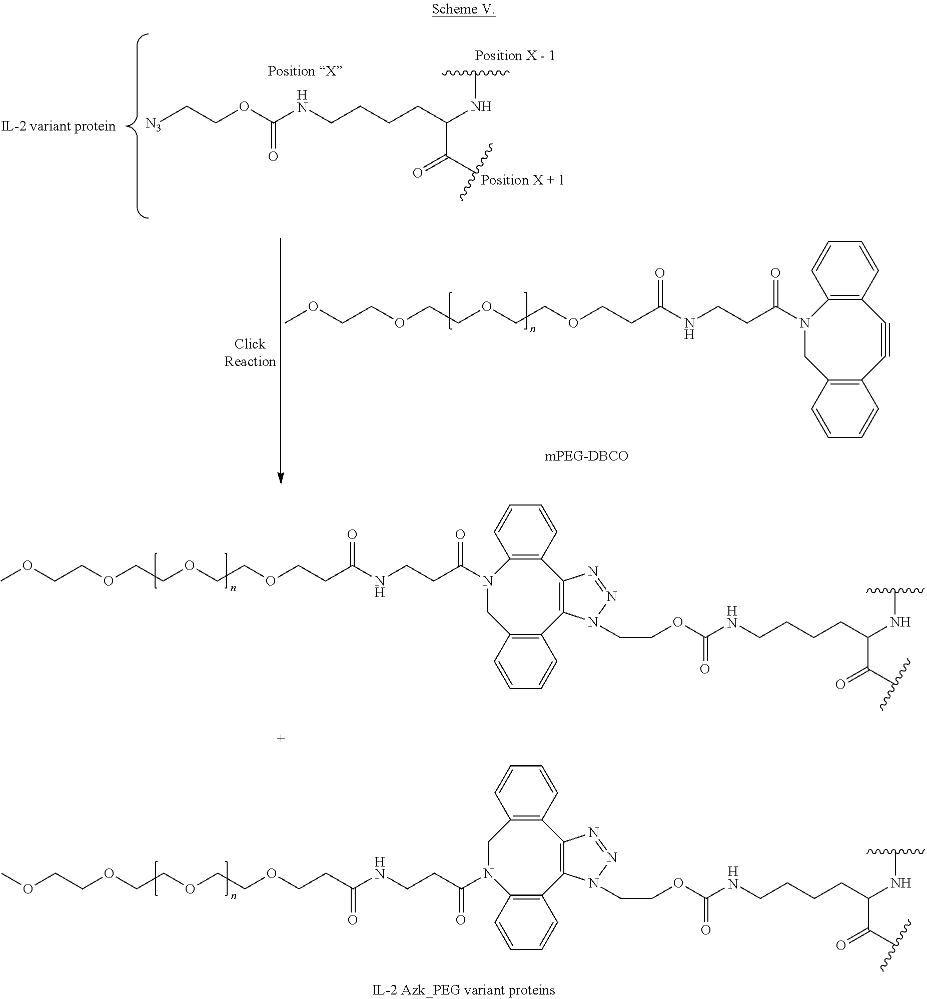

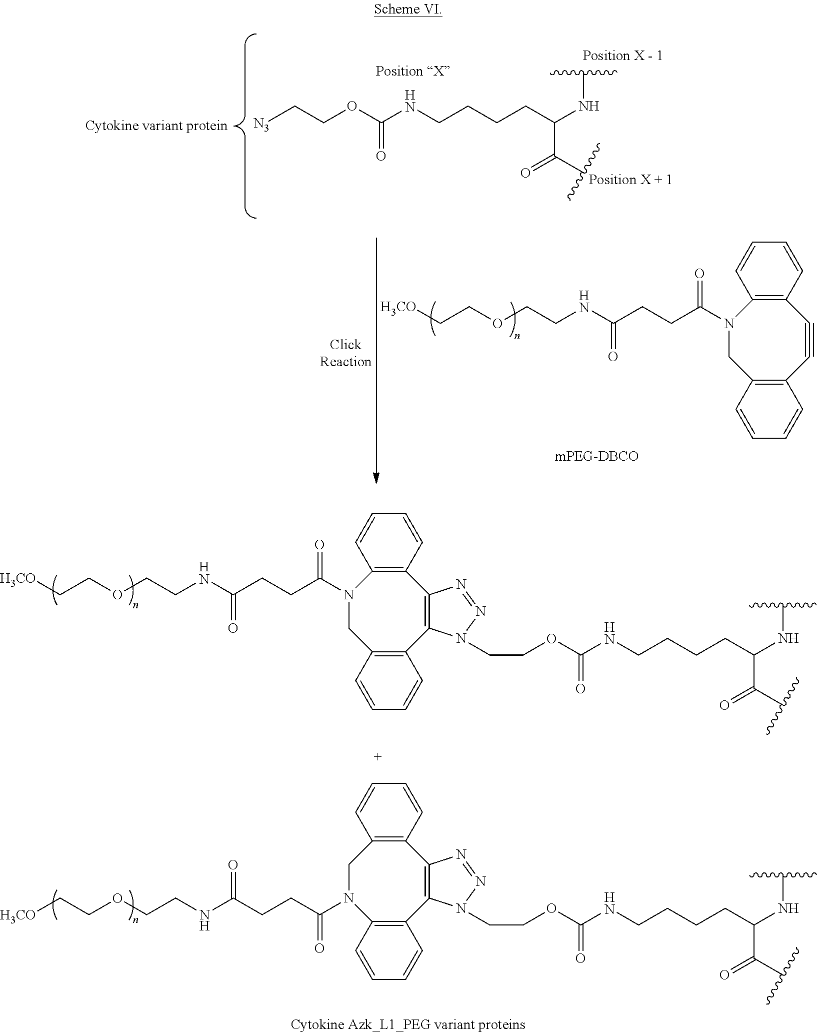

Conjugation reactions such as a click reaction described herein may generate a single regioisomer, or a mixture of regioisomers. In some instances the ratio of regioisomers is about 1:1. In some instances the ratio of regioisomers is about 2:1. In some instances the ratio of regioisomers is about 1.5:1. In some instances the ratio of regioisomers is about 1.2:1. In some instances the ratio of regioisomers is about 1.1:1. In some instances the ratio of regioisomers is greater than 1:1.

### IL-2 Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., Gram-positive bacterium or a Gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293 FT cell line, 293F cells, 293 H cells, A549 cells, MDCK cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REX™-CHO cell line, and T-REX™-HeLa cell line.

In some embodiments, a eukaryotic host cell is an insect host cell. Exemplary insect host cells include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris (K. phaffii) vector, pGAPZA, B, & C Pichia pastoris (K. phaffii) vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of an IL-2 polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, RNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631, the disclosure of each of which is incorporated herein by reference. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, an IL-2 polypeptide is generated as a circularly permuted form, either via an expression host system or through a cell-free system.

### Production of Cytokine Polypeptide Comprising an Unnatural Amino Acid

An orthogonal or expanded genetic code can be used in the present disclosure, in which one or more specific codons present in the nucleic acid sequence of an IL-2 polypeptide are allocated to encode the unnatural amino acid so that it can be genetically incorporated into the IL-2 by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464 (7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113 (38): E5588-5597 (2016), the disclosure of which is incorporated herein by reference. In some cases, the recoded codon is as described in Ostrov et al., “Design, synthesis, and testing toward a 57-codon genome,” Science 353 (6301): 819-822 (2016), the disclosure of which is incorporated herein by reference.

In some instances, unnatural nucleic acids are utilized leading to incorporation of one or more unnatural amino acids into the IL-2. Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine ([5,4-b][1,4]benzoxazin-2 (3H)-one), phenothiazine cytidine (1H-pyrimido[5,4-b][1,4]benzothiazin-2 (3H)-one), G-clamps, phenoxazine cytidine (e.g. 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J.I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993; the disclosure of each of which is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo[2,3-d]pyrimidin-5-yl, 2-amino-4-oxopyrolo[2,3-d]pyrimidin-5-yl, 2-amino-4-oxopyrrolo[2,3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050; the disclosure of each of which is incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10 (1-3), 339-343; Leonid et al., 1995, 14 (3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337); the disclosure of each of which is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′—OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19 (8), 1029-1033). Unnatural nucleic acids can include 5′-phosphonate deoxyribonucleoside monomers and dimers having a 5′-phosphate group (Nawrot et al., Oligonucleotides, 2006, 16 (1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105 (26), 7638-7640; and Hampton et al., J. Am. Chem. Soc, 1973, 95 (13), 4404-4414). The disclosure of each reference listed in this paragraph is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181, the disclosure of each of which is incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group.

The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl, 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is incorporated herein by reference.

Examples of nucleic acids having modified sugar moieties include, without limitation, nucleic acids comprising 5′-vinyl, 5′-methyl (R or S), 4′-S, 2′-F, 2′—OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129 (26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393. The disclosure of each reference listed in this paragraph is incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24:1841-1848; Chaturvedi et al., 1996, Nucleic Acids Res. 24:2318-2323; Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8:1157-1179; the disclosure of each of which is incorporated herein by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8:1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574, 142; 5,585,481; 5,587,371; 5,595,726; 5,597,696; 5,599,923; 5,599,928 and 5,688,941. The disclosure of each reference listed in this paragraph is incorporated herein by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

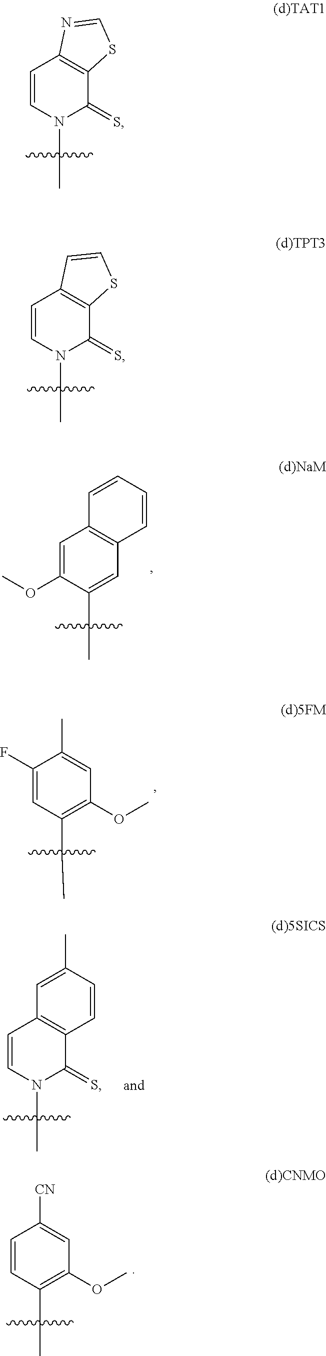 [SMILES: *n1ccc2ncsc2c1=S ; *n1ccc2ccsc2c1=S ; *c1cc2ccccc2cc1OC ; *c1cc(F)c(C)cc1OC ; *n1ccc2cc(C)ccc2c1=S ; *c1ccc(C#N)cc1OC]

Exemplary unnatural base pairs include: (d)TPT3-(d)NaM; (d)5SICS-(d)NaM; (d)CNMO-(d)TAT1; (d)NaM-(d)TAT1; (d)CNMO-(d)TPT3; and (d)5FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is incorporated herein by reference. In some embodiments, unnatural nucleotides include:

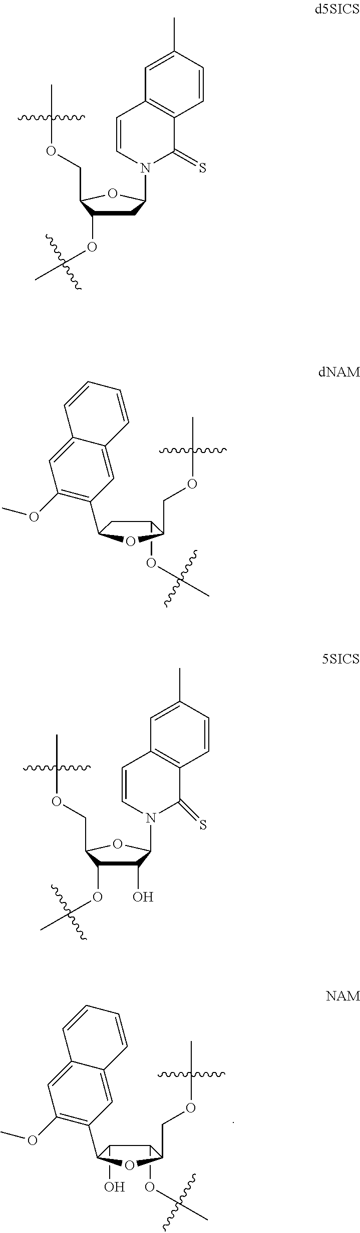

In some embodiments, the unnatural nucleotides that may be used to prepare the IL-2 conjugates disclosed herein may be derived from a compound of the formula

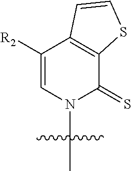

wherein R² is selected from the group consisting of hydrogen, alkyl, alkenyl, alkynyl, methoxy, methanethiol, methaneseleno, halogen, cyano, and azido; and
the wavy line indicates a bond to a ribosyl or 2′-deoxyribosyl, wherein the 5′-hydroxy group of the ribosyl or 2′-deoxyribosyl moiety is in free form, is connected to a monophosphate, diphosphate, triphosphate, α-thiotriphosphate, β-thiotriphosphate, or γ-thiotriphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

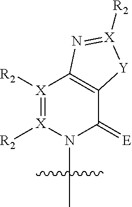

wherein:
each X is independently carbon or nitrogen;
R₂ is absent when X is nitrogen, and is present when X is carbon and is independently hydrogen, alkyl, alkenyl, alkynyl, methoxy, methanethiol, methaneseleno, halogen, cyano, or azide;
Y is sulfur, oxygen, selenium, or secondary amine;
E is oxygen, sulfur, or selenium; and
the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R₂ is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂—CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

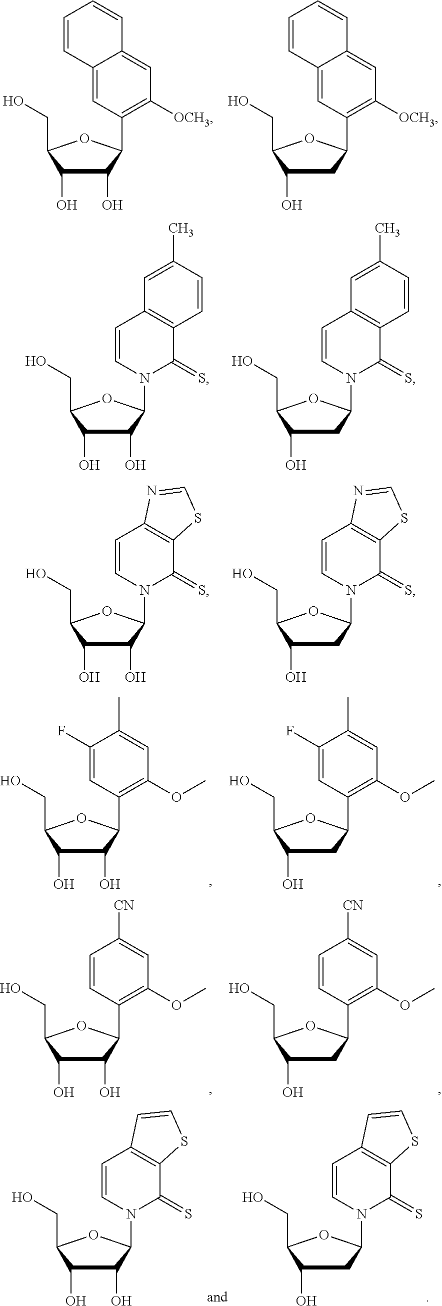

In some embodiments, the unnatural nucleotides that may be used to prepare the IL-2 conjugates disclosed herein include

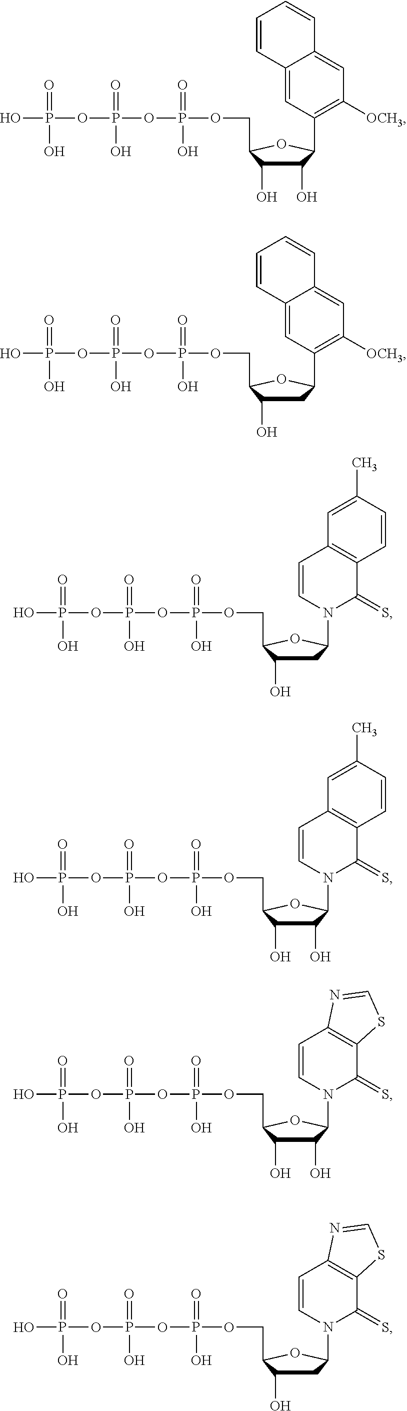

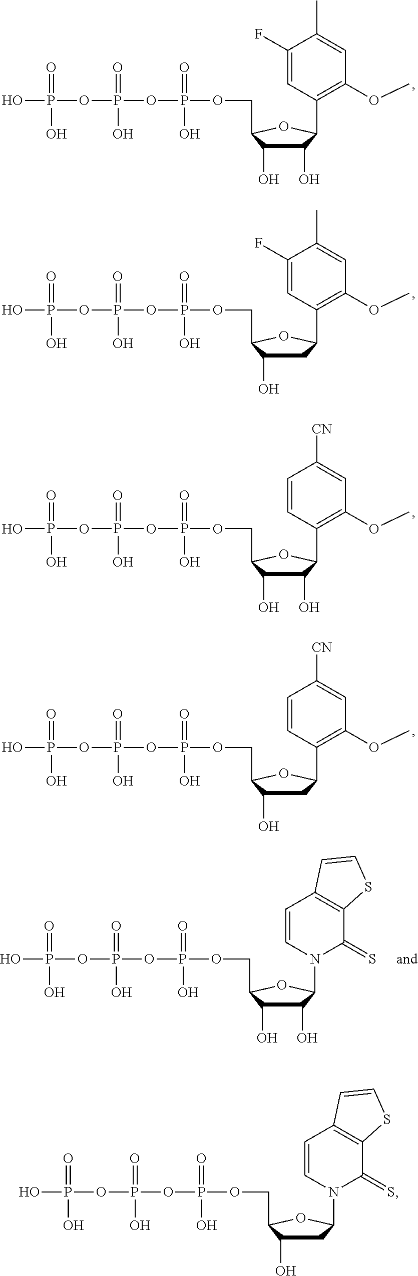

or salts thereof.

In some embodiments, an unnatural base pair generate an unnatural amino acid described in Dumas et al., “Designing logical codon reassignment-Expanding the chemistry in biology,” Chemical Science, 6:50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNACUA pairs, E. coli LeuRS (Ec-Leu)/B. stearothermophilus tRNACUA pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such as p-aminophenylalanine and p-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the cytokine (e.g., the IL polypeptide) by a Ec-Tyr/tRNACUA or a Ec-Leu/tRNACUA pair. Exemplary UAAs that can be incorporated by a Ec-Tyr/tRNACUA or a Ec-LeultRNACUA pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido) hexanoic acid, N-ε-D-prolyl-L-lysine, and N-8-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; and N-ε-(1-methylcyclopro-2-enecarboxamido) lysine. In some embodiments, the IL-2 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PyIRS). Other methods are known to those of ordinary skill in the art, such as those disclosed in Zhang et al., Nature 2017, 551 (7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosure of each of which is incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551 (7682): 644-647; Malyshev et al. Nature 2014 (509 (7500), 385-388; and Zhang et al. Proc Natl Acad Sci USA, 2017, 114:1317-1322.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNACUA pairs, E. coli LeuRS (Ec-Leu)/B. stearothermophilus tRNACUA pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is incorporated herein by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PyIRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cell is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at position 64 of the sequence that encodes the protein having SEQ ID NO: 1, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

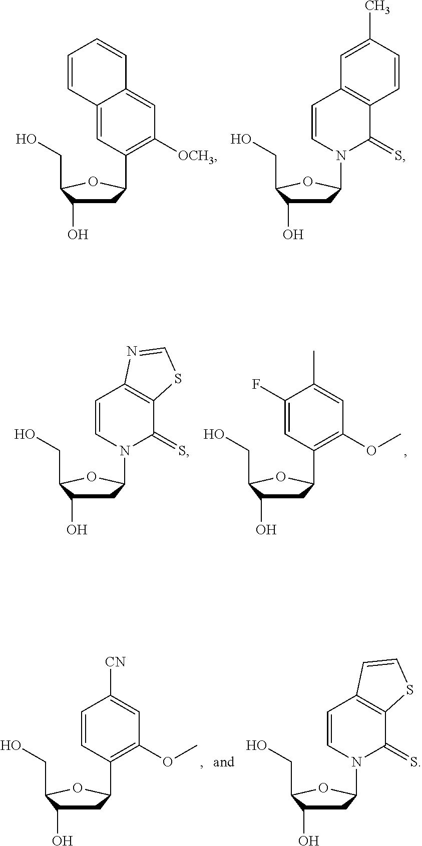

In some embodiments, the first and second unnatural nucleotides comprising the unnatural base pair in the double-stranded oligonucleotide may be derived from

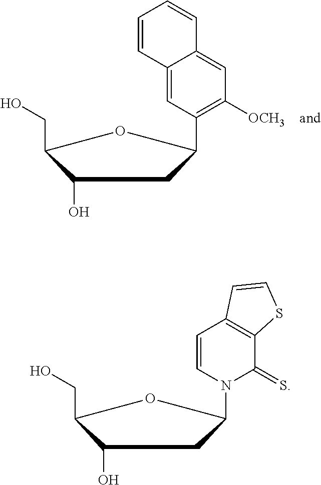

In some embodiments, the triphosphates of the first and second unnatural nucleotides include,

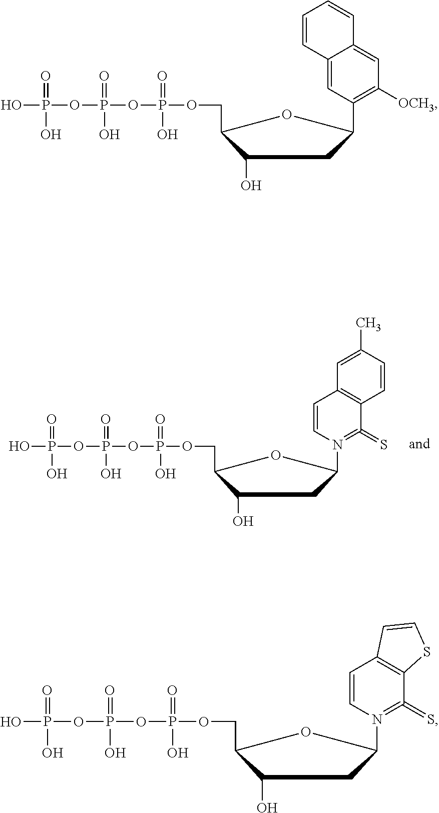

or salts thereof. In some embodiments, the triphosphates of the first and second unnatural nucleotides include,

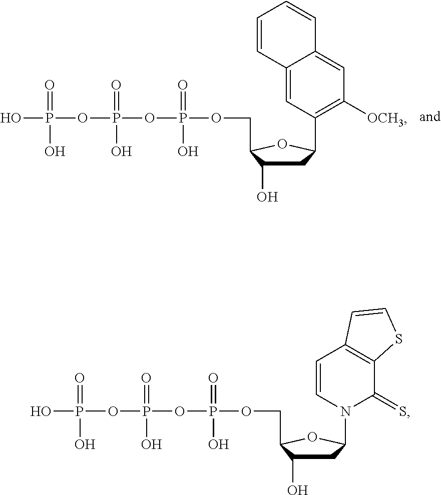

or salts thereof. In some embodiments, the mRNA derived the double-stranded oligonucleotide comprising a first unnatural nucleotide and a second unnatural nucleotide may comprise a codon comprising an unnatural nucleotide derived from

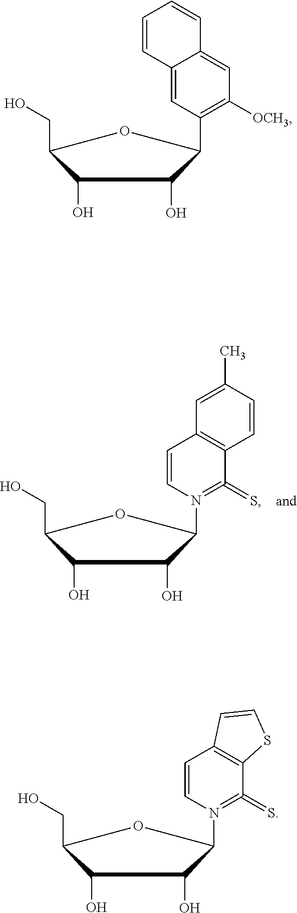

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

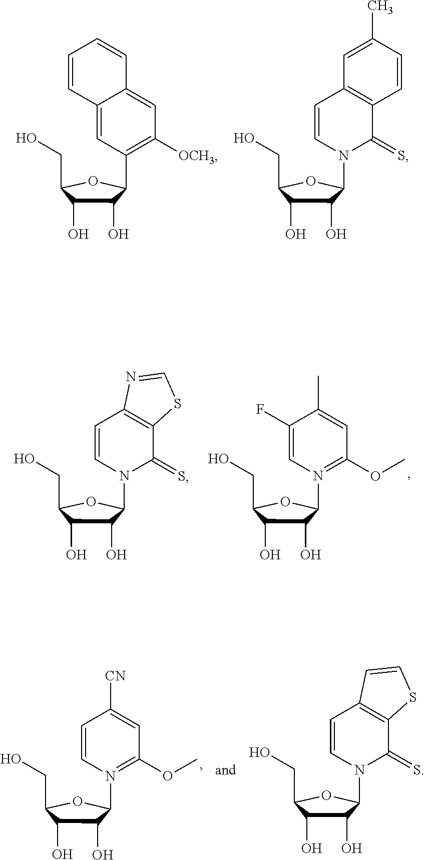

In some embodiments, the mRNA comprises an unnatural nucleotide derived from

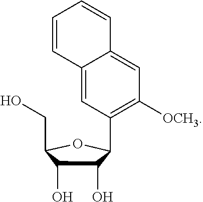

In some embodiments, the mRNA comprises an unnatural nucleotide derived from

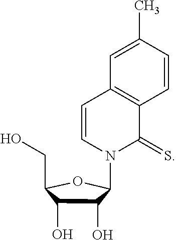

In some embodiments, the mRNA comprises an unnatural nucleotide derived from

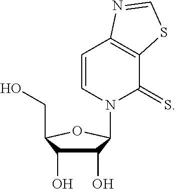

In some embodiments, the mRNA comprises an unnatural nucleotide derived from

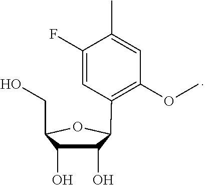

In some embodiments, the mRNA comprises an unnatural nucleotide derived from

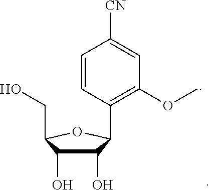

In some embodiments, the mRNA comprises an unnatural nucleotide derived from

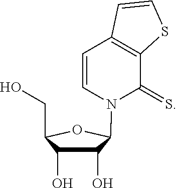

In some embodiments, the tRNA comprises an unnatural nucleotide derived from

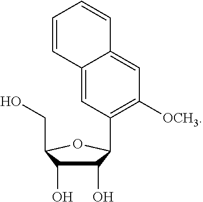

In some embodiments, the tRNA comprises an unnatural nucleotide derived from

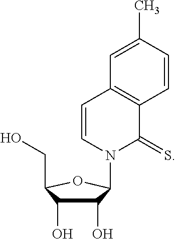

In some embodiments, the tRNA comprises an unnatural nucleotide derived from

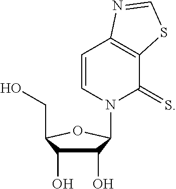

In some embodiments, the tRNA comprises an unnatural nucleotide derived from

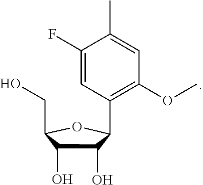

In some embodiments, the tRNA comprises an unnatural nucleotide derived from

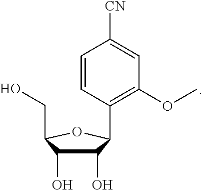

In some embodiments, the tRNA comprises an unnatural nucleotide derived from

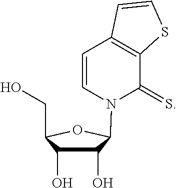

In some embodiments, the mRNA comprises an unnatural nucleotide derived from

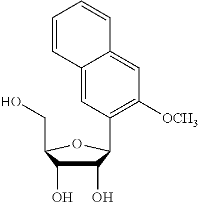

and the tRNA comprises an unnatural nucleotide derived from

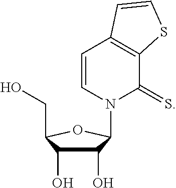

In some embodiments, the mRNA comprises an unnatural nucleotide derived from

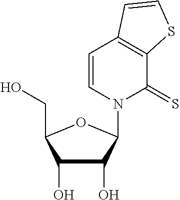

and the tRNA comprises an unnatural nucleotide derived from

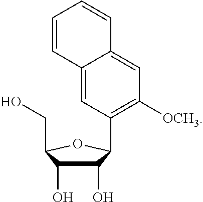

In some embodiments, the mRNA comprises an unnatural nucleotide derived from

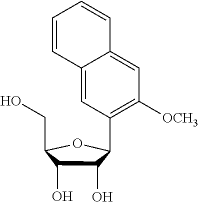

and the tRNA comprises an unnatural nucleotide derived from

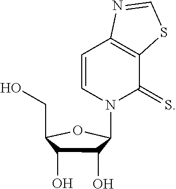

In some embodiments, the mRNA comprises an unnatural nucleotide derived from

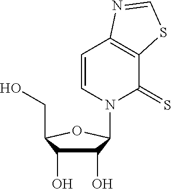

and the tRNA comprises an unnatural nucleotide derived from

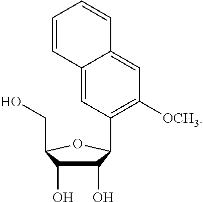

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551 (7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551 (7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, an IL-2 polypeptide comprising an unnatural amino acid(s) is prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the IL-2 polypeptide. Alternatively, two or more unnatural amino acids may be incorporated into the IL-2 polypeptide at two or more sites in the protein.

Once the IL-2 polypeptide incorporating the unnatural amino acid(s) has been produced in the host cell it can be extracted therefrom by a variety of techniques known in the art, including enzymatic, chemical and/or osmotic lysis and physical disruption. The IL-2 polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21 (DE3)), but most suitably host cells are eukaryotic cells, for example insect cells (e.g., Drosophila such as Drosophila melanogaster), yeast cells, nematodes (e.g., C. elegans), mice (e.g., Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell-such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### Chimeric Antigen Receptor Therapies

In one embodiment, a chimeric antigen receptor (CAR) therapy described in any of the methods provided herein comprises T cells expressing a CAR. In one embodiment, a CAR therapy described in any of the methods provided herein comprises gamma delta T cells expressing a CAR. In one embodiment, a CAR therapy described in any of the methods provided herein comprises natural killer (NK) T cells expressing a CAR. In one embodiment, a CAR therapy described in any of the methods provided herein comprises natural killer (NK) cells expressing a CAR. In one embodiment, the CAR binds CD19, CD22, CD19 and CD22, or B-cell maturation antigen (BCMA). In one embodiment, the CAR binds CD20, Claudin-6, or Her2. In some embodiments, immune cells used in a CAR therapy described herein can be autologous. In some embodiments, immune cells used in a CAR therapy described herein can be allogeneic. In some embodiments, immune cells used in a CAR therapy described herein can be “off-the-shelf” immune cells.

In one embodiment, the CAR (e.g., expressed by a T cell, a gamma delta T cell, an NK T cell, or an NK cell) comprises an anti-CD19 domain comprising an anti-CD19 heavy chain variable domain (VH) and an anti-CD19 light chain variable domain (VL). In one embodiment, the anti-CD19 VH comprises a CDR-H1 comprising the amino acid sequence of SEQ ID NO: 3, a CDR-H2 comprising the amino acid sequence of SEQ ID NO: 4, and a CDR-H3 comprising the amino acid sequence of SEQ ID NO: 5. In one embodiment, the anti-CD19 VH comprises an amino acid sequence at least 80%, 85%, 90%, 95%, 96%, 97%, 98%, or 99% identical to the amino acid sequence of SEQ ID NO: 6. In one embodiment, the anti-CD19 VH comprises the amino acid sequence of SEQ ID NO: 6. In one embodiment, the anti-CD19 VL comprises a CDR-L1 comprising the amino acid sequence of SEQ ID NO: 7, a CDR-L2 comprising the amino acid sequence of SEQ ID NO: 8, and a CDR-L3 comprising the amino acid sequence of SEQ ID NO: 9. In one embodiment, the anti-CD19 VL comprises an amino acid sequence at least 80%, 85%, 90%, 95%, 96%, 97%, 98%, or 99% identical to the amino acid sequence of SEQ ID NO: 10. In one embodiment, the anti-CD19 VL comprises the amino acid sequence of SEQ ID NO: 10.

In one embodiment, the CAR (e.g., expressed by a T cell, a gamma delta T cell, an NK T cell, or an NK cell) comprises an anti-CD19 domain, e.g., comprising an anti-CD19 VH and an anti-CD19 VL. In one embodiment, the anti-CD19 VH comprises a CDR-H1 comprising the amino acid sequence of SEQ ID NO: 11, a CDR-H2 comprising the amino acid sequence of SEQ ID NO: 12, and a CDR-H3 comprising the amino acid sequence of SEQ ID NO: 13. In one embodiment, the anti-CD19 VH comprises an amino acid sequence at least 80%, 85%, 90%, 95%, 96%, 97%, 98%, or 99% identical to the amino acid sequence of SEQ ID NO: 14. In one embodiment, the anti-CD19 VH comprises the amino acid sequence of SEQ ID NO: 14. In one embodiment, the anti-CD19 VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17. In one embodiment, the anti-CD19 VL comprises an amino acid sequence at least 80%, 85%, 90%, 95%, 96%, 97%, 98%, or 99% identical to the amino acid sequence of SEQ ID NO: 18. In one embodiment, the anti-CD19 VL comprises the amino acid sequence of SEQ ID NO: 18.

In one embodiment, the CAR (e.g., expressed by a T cell, a gamma delta T cell, an NK T cell, or an NK cell) comprises an anti-CD19 domain comprising an anti-CD19 VH and an anti-CD19 VL. In one embodiment, the anti-CD19 VH comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21. In one embodiment, the anti-CD19 VH comprises an amino acid sequence at least 80%, 85%, 90%, 95%, 96%, 97%, 98%, or 99% identical to the amino acid sequence of SEQ ID NO: 22. In one embodiment, the anti-CD19 VH comprises the amino acid sequence of SEQ ID NO: 22. In one embodiment, the anti-CD19 VL comprises a CDR-L1 comprising the amino acid sequence of SEQ ID NO: 23, a CDR-L2 comprising the amino acid sequence of SEQ ID NO: 24, and a CDR-L3 comprising the amino acid sequence of SEQ ID NO: 25. In one embodiment, the anti-CD19 VL comprises an amino acid sequence at least 80%, 85%, 90%, 95%, 96%, 97%, 98%, or 99% identical to the amino acid sequence of SEQ ID NO: 26. In one embodiment, the anti-CD19 VL comprises the amino acid sequence of SEQ ID NO: 26.

In one embodiment, in addition to or in lieu of comprising an anti-CD19 domain, the CAR (e.g., expressed by a T cell, a gamma delta T cell, an NK T cell, or an NK cell) comprises an anti-CD22 domain, e.g., comprising an anti-CD22 VH and an anti-CD22 VL. In one embodiment, the anti-CD22 VH comprises a CDR-H1 comprising the amino acid sequence of SEQ ID NO: 27, a CDR-H2 comprising the amino acid sequence of SEQ ID NO: 28, and a CDR-H3 comprising the amino acid sequence of SEQ ID NO: 29. In one embodiment, the anti-CD22 VH comprises an amino acid sequence at least 80%, 85%, 90%, 95%, 96%, 97%, 98%, or 99% identical to the amino acid sequence of SEQ ID NO: 30. In one embodiment, the anti-CD22 VH comprises the amino acid sequence of SEQ ID NO: 30. In one embodiment, the anti-CD22 VL comprises a CDR-L1 comprising the amino acid sequence of SEQ ID NO: 31, a CDR-L2 comprising the amino acid sequence of SEQ ID NO: 32, and a CDR-L3 comprising the amino acid sequence of SEQ ID NO: 33. In one embodiment, the anti-CD22 VL comprises an amino acid sequence at least 80%, 85%, 90%, 95%, 96%, 97%, 98%, or 99% identical to the amino acid sequence of SEQ ID NO: 34. In one embodiment, the anti-CD22 VL comprises the amino acid sequence of SEQ ID NO: 34.

In one embodiment, in lieu of comprising an anti-CD19 domain or an anti-CD22 domain, the CAR (e.g., expressed by a T cell, a gamma delta T cell, an NK T cell, or an NK cell) comprises an anti-B-cell maturation antigen (BCMA) domain, e.g., comprising an anti-BCMA VH and an anti-BCMA VL, or comprising a first anti-BCMA single domain antibody (V_{H}H) and/or a second anti-BCMA V_{H}H. In one embodiment, the anti-BCMA VH comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 51. In one embodiment, the anti-BCMA VH comprises an amino acid sequence at least 80%, 85%, 90%, 95%, 96%, 97%, 98%, or 99% identical to the amino acid sequence of SEQ ID NO: 52. In one embodiment, the anti-BCMA VH comprises the amino acid sequence of SEQ ID NO: 52. In one embodiment, the anti-BCMA VL comprises a CDR-L1 comprising the amino acid sequence of SEQ ID NO: 53, a CDR-L2 comprising the amino acid sequence of SEQ ID NO: 54, and a CDR-L3 comprising the amino acid sequence of SEQ ID NO: 55. In one embodiment, the anti-BCMA VL comprises an amino acid sequence at least 80%, 85%, 90%, 95%, 96%, 97%, 98%, or 99% identical to the amino acid sequence of SEQ ID NO: 56. In one embodiment, the anti-BCMA VL comprises the amino acid sequence of SEQ ID NO: 56. In one embodiment, the CAR comprises an anti-BCMA domain comprising a first anti-BCMA V_{H}H and/or a second anti-BCMA V_{H}H. In one embodiment, the first anti-BCMA V_{H}H comprises a CDR-H1 comprising the amino acid sequence of SEQ ID NO: 59, a CDR-H2 comprising the amino acid sequence of SEQ ID NO: 60, and a CDR-H3 comprising the amino acid sequence of SEQ ID NO: 61. In one embodiment, the first anti-BCMA V_{H}H comprises an amino acid sequence at least 80%, 85%, 90%, 95%, 96%, 97%, 98%, or 99% identical to the amino acid sequence of SEQ ID NO: 62. In one embodiment, the first anti-BCMA V_{H}H comprises the amino acid sequence of SEQ ID NO: 62. In one embodiment, the second anti-BCMA V_{H}H comprises a CDR-H1 comprising the amino acid sequence of SEQ ID NO: 63, a CDR-H2 comprising the amino acid sequence of SEQ ID NO: 64, and a CDR-H3 comprising the amino acid sequence of SEQ ID NO: 65. In one embodiment, the second anti-BCMA V_{H}H comprises an amino acid sequence at least 80%, 85%, 90%, 95%, 96%, 97%, 98%, or 99% identical to the amino acid sequence of SEQ ID NO: 66. In one embodiment, the second anti-BCMA V_{H}H comprises the amino acid sequence of SEQ ID NO: 66.

In one embodiment, the CAR (e.g., expressed by a T cell, a gamma delta T cell, an NK T cell, or an NK cell) comprises a CD8a chain, e.g., comprising an amino acid sequence at least 80%, 85%, 90%, 95%, 96%, 97%, 98%, or 99% identical to SEQ ID NO: 35, 36, 69, or 70. In one embodiment, the CAR comprises a CD8a chain, comprising the amino acid sequence of SEQ ID NO: 35, 36, 69, or 70. In one embodiment, the CAR comprises a 4-1BB domain, comprising an amino acid sequence at least 80%, 85%, 90%, 95%, 96%, 97%, 98%, or 99% identical to SEQ ID NO: 37 or 38. In one embodiment, the CAR comprises a 4-1BB domain, comprising the amino acid sequence of SEQ ID NO: 37 or 38. In one embodiment, the CAR comprises a CD28 domain, comprising an amino acid sequence at least 80%, 85%, 90%, 95%, 96%, 97%, 98%, or 99% identical to SEQ ID NO: 39. In one embodiment, the CAR comprises a CD28 domain, comprising the amino acid sequence of SEQ ID NO: 39. In one embodiment, the CAR comprises a CD137 domain, comprising an amino acid sequence at least 80%, 85%, 90%, 95%, 96%, 97%, 98%, or 99% identical to SEQ ID NO: 71. In one embodiment, the CAR comprises a CD137 domain, comprising the amino acid sequence of SEQ ID NO: 71. In one embodiment, the CAR comprises a CD32 domain, comprising an amino acid sequence at least 80%, 85%, 90%, 95%, 96%, 97%, 98%, or 99% identical to SEQ ID NO: 40, 41, 42, 72, or 73. In one embodiment, the CAR comprises a CD35 domain, comprising the amino acid sequence of SEQ ID NO: 40, 41, 42, 72, or 73.

In one embodiment, the CAR (e.g., expressed by a T cell, a gamma delta T cell, an NK T cell, or an NK cell) is an anti-CD19 CAR. In one embodiment, the anti-CD19 CAR comprises an amino acid sequence at least 80%, 85%, 90%, 95%, 96%, 97%, 98%, or 99% identical to the amino acid sequence of SEQ ID NO: 45. In one embodiment, the anti-CD19 CAR comprises the amino acid sequence of SEQ ID NO: 45. In one embodiment, the anti-CD19 CAR is encoded by a nucleic acid sequence at least 80%, 85%, 90%, 95%, 96%, 97%, 98%, or 99% identical to the nucleic acid sequence of SEQ ID NO: 46. In one embodiment, the anti-CD19 CAR is encoded by the nucleic acid sequence of SEQ ID NO: 46.

In one embodiment, the CAR (e.g., expressed by a T cell, a gamma delta T cell, an NK T cell, or an NK cell) is an anti-CD19 CAR. In one embodiment, the anti-CD19 CAR comprises an amino acid sequence at least 80%, 85%, 90%, 95%, 96%, 97%, 98%, or 99% identical to the amino acid sequence of SEQ ID NO: 47. In one embodiment, the anti-CD19 CAR comprises the amino acid sequence of SEQ ID NO: 47. In one embodiment, the anti-CD19 CAR is encoded by a nucleic acid sequence at least 80%, 85%, 90%, 95%, 96%, 97%, 98%, or 99% identical to the nucleic acid sequence of SEQ ID NO: 48. In one embodiment, the anti-CD19 CAR is encoded by the nucleic acid sequence of SEQ ID NO: 48.

In one embodiment, the CAR (e.g., expressed by a T cell, a gamma delta T cell, an NK T cell, or an NK cell) is an anti-CD19/CD22 CAR. In one embodiment, the anti-CD19/CD22 CAR comprises an amino acid sequence at least 80%, 85%, 90%, 95%, 96%, 97%, 98%, or 99% identical to the amino acid sequence of SEQ ID NO: 43. In one embodiment, the anti-CD19/CD22 CAR comprises the amino acid sequence of SEQ ID NO: 43. In one embodiment, the anti-CD19/CD22 CAR is encoded by a nucleic acid sequence at least 80%, 85%, 90%, 95%, 96%, 97%, 98%, or 99% identical to the nucleic acid sequence of SEQ ID NO: 44. In one embodiment, the anti-CD19/CD22 CAR is encoded by the nucleic acid sequence of SEQ ID NO: 44.

In one embodiment, the CAR (e.g., expressed by a T cell, a gamma delta T cell, an NK T cell, or an NK cell) is an anti-BCMA CAR. In one embodiment, the anti-BCMA CAR comprises an amino acid sequence at least 80%, 85%, 90%, 95%, 96%, 97%, 98%, or 99% identical to the amino acid sequence of SEQ ID NO: 57. In one embodiment, the anti-BCMA CAR comprises the amino acid sequence of SEQ ID NO: 57. In one embodiment, the anti-BCMA CAR is encoded by a nucleic acid sequence at least 80%, 85%, 90%, 95%, 96%, 97%, 98%, or 99% identical to the nucleic acid sequence of SEQ ID NO: 58. In one embodiment, the anti-BCMA CAR is encoded by the nucleic acid sequence of SEQ ID NO: 58.

In one embodiment, the CAR (e.g., expressed by a T cell, a gamma delta T cell, an NK T cell, or an NK cell) is an anti-BCMA CAR. In one embodiment, the anti-BCMA CAR comprises an amino acid sequence at least 80%, 85%, 90%, 95%, 96%, 97%, 98%, or 99% identical to the amino acid sequence of SEQ ID NO: 67. In one embodiment, the anti-BCMA CAR comprises the amino acid sequence of SEQ ID NO: 67. In one embodiment, the anti-BCMA CAR is encoded by a nucleic acid sequence at least 80%, 85%, 90%, 95%, 96%, 97%, 98%, or 99% identical to the nucleic acid sequence of SEQ ID NO: 68. In one embodiment, the anti-BCMA CAR is encoded by the nucleic acid sequence of SEQ ID NO: 68.

In one embodiment, the CAR therapy comprises 0.2-5.0×10⁶ or 0.5-1.0×10⁶ of the cells per kg body weight of the subject, e.g., as a single dose. In one embodiment, the CAR therapy comprises 0.2-5.0×10⁶ of the cells per kg body weight of the subject, e.g., as a single dose. In one embodiment, the CAR therapy comprises 0.5-1.0×10⁶ of the cells per kg body weight of the subject, e.g., as a single dose. In one embodiment, the CAR therapy comprises 1.0×10⁶ or 2.0×10⁶ of the cells per kg body weight of the subject, e.g., as a single dose. In one embodiment, the CAR therapy comprises 1.0×10⁶ of the cells per kg body weight of the subject, e.g., as a single dose. In one embodiment, the CAR therapy comprises 2.0×10⁶ of the cells per kg body weight of the subject, e.g., as a single dose. In one embodiment, the CAR therapy comprises 0.1-2.5×10⁸ or 0.6-6.0×10⁸ of the cells, e.g., as a single dose. In one embodiment, the CAR therapy comprises 0.1-2.5×10⁸ of the cells, e.g., as a single dose. In one embodiment, the CAR therapy comprises 0.6-6.0×10⁸ of the cells, e.g., as a single dose. In one embodiment, the CAR therapy comprises 0.9-1.1×10⁸, 0.5-1.1×10⁸, or 3.0-4.6×10⁸ of the cells, e.g., as a single dose. In one embodiment, the CAR therapy comprises 0.9-1.1×10⁸ of the cells. In one embodiment, the CAR therapy comprises 0.5-1.1×10⁸ of the cells, e.g., as a single dose. In one embodiment, the CAR therapy comprises 3.0-4.6×10⁸ of the cells, e.g., as a single dose.

### Methods of Treatment

In one aspect, provided herein is a method of treating a cancer in a subject in need thereof, comprising administering to the subject (a) an IL-2 conjugate as described herein, and (b) a chimeric antigen receptor (CAR) therapy.

In one aspect, the cancer is leukemia, myeloma, or lymphoma. In one aspect, the cancer is leukemia. In one aspect, the leukemia is acute lymphoblastic leukemia or chronic lymphocytic leukemia. In one aspect, the cancer is a myeloma. In one aspect, the myeloma is multiple myeloma. In one aspect, the lymphoma is non-Hodgkin lymphoma, follicular lymphoma, transformed follicular lymphoma, mantle cell lymphoma, primary mediastinal B-cell lymphoma, Burkitt's lymphoma, or diffuse large B-cell lymphoma (DLBCL). In one aspect, the lymphoma is Burkitt's lymphoma. In one aspect, the lymphoma is DLBCL. In one aspect, the cancer is a solid tumor, melanoma, or glioblastoma.

In one aspect, provided herein is a method of treating diffuse large B-cell lymphoma (DLBCL) in a subject in need thereof, comprising administering to the subject an IL-2 conjugate as described herein. In some embodiments, the method of treating DLBCL in a subject in need thereof comprises administering to the subject about 8 μg/kg IL-2 as an IL-2 conjugate as described herein. In some embodiments, the method of treating DLBCL in a subject in need thereof comprises administering to the subject about 16 μg/kg IL-2 as an IL-2 conjugate as described herein. In some embodiments, the method of treating DLBCL in a subject in need thereof comprises administering to the subject about 24 μg/kg IL-2 as an IL-2 conjugate as described herein. In some embodiments, the method of treating DLBCL in a subject in need thereof comprises administering to the subject about 32 μg/kg IL-2 as an IL-2 conjugate as described herein. In some embodiments, the method of treating DLBCL in a subject in need thereof further comprises administering to the subject a CAR therapy.

In a further aspect, provided herein is use of an IL-2 conjugate for the manufacture of a medicament for a method disclosed herein of treating cancer, including DLBCL, in a subject in need thereof.

The embodiments described in the following sections apply to any of the foregoing aspects.

### Administration

In some embodiments, the IL-2 conjugate is administered as at least a third or subsequent line of therapy.

In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to the subject by intravenous, subcutaneous, or intramuscular administration. In some embodiments, the IL-2 conjugate is administered to the subject by intravenous administration. In some embodiments, the IL-2 conjugate is administered to the subject by subcutaneous administration. In some embodiments, the IL-2 conjugate is administered to the subject by intramuscular administration. In some embodiments, the IL-2 conjugate is administered to the subject by intravenous administration.

The IL-2 conjugate may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate is administered to a subject in need thereof about once every week, about once every two weeks, about once every three weeks, or about once every 4 weeks. In some embodiments, the IL-2 conjugate is administered to a subject in need thereof once every week. In some embodiments, the IL-2 conjugate is administered to a subject in need thereof once every two weeks. In some embodiments, the IL-2 conjugate is administered to a subject in need thereof once every three weeks. In some embodiments, the IL-2 conjugate is administered to a subject in need thereof once every 4 weeks. In some embodiments, the IL-2 conjugate is administered about once every 14, 15, 16, 17, 18, 19, 20, or 21 days.

In some embodiments, the CAR therapy is administered to the subject by intravenous administration. In some embodiments, the CAR therapy is administered to the subject before any dose of the IL-2 conjugate is administered to the subject. In some embodiments, the CAR therapy is administered to the subject at least about one week before any dose of the IL-2 conjugate is administered to the subject. In some embodiments, the CAR therapy is administered to the subject at least about two weeks before any dose of the IL-2 conjugate is administered to the subject. In some embodiments, the CAR therapy is administered to the subject at least about three weeks before any dose of the IL-2 conjugate is administered to the subject. In some embodiments, the CAR therapy is administered to the subject at least about four weeks before any dose of the IL-2 conjugate is administered to the subject. In some embodiments, the CAR therapy is administered to the subject at least about five weeks before any dose of the IL-2 conjugate is administered to the subject. In some embodiments, the CAR therapy is administered to the subject at least about six weeks before any dose of the IL-2 conjugate is administered to the subject. In some embodiments, the CAR therapy is administered to the subject at least about seven weeks before any dose of the IL-2 conjugate is administered to the subject. In some embodiments, the CAR therapy is administered to the subject at least about eight weeks before any dose of the IL-2 conjugate is administered to the subject. In some embodiments, the CAR therapy is administered to the subject at least about nine weeks before any dose of the IL-2 conjugate is administered to the subject. In some embodiments, the CAR therapy is administered to the subject at least about ten weeks before any dose of the IL-2 conjugate is administered to the subject. In some embodiments, the CAR therapy is administered to the subject at least about 11 weeks before any dose of the IL-2 conjugate is administered to the subject. In some embodiments, the CAR therapy is administered to the subject at least about 12 weeks before any dose of the IL-2 conjugate is administered to the subject. In some embodiments, the CAR therapy is administered to the subject at least about 13 weeks before any dose of the IL-2 conjugate is administered to the subject. In some embodiments, the CAR therapy is administered to the subject at least about 14 weeks before any dose of the IL-2 conjugate is administered to the subject. In some embodiments, the CAR therapy is administered to the subject at least about 100 days before any dose of the IL-2 conjugate is administered to the subject. In some embodiments, the CAR therapy is administered to the subject about 1-10, 10-20, 20-30, 30-40, 40-50, 50-60, 60-70, 70-80, 80-90, or 90-100 days before any dose of the IL-2 conjugate is administered to the subject. In some embodiments, the CAR therapy is administered to the subject about 1-2, 2-3, 3-4, 4-5, 5-6, 6-7, 7-8, 8-9, 9-10, 10-11, 11-12, 12-13, 13-14, or 14-15 weeks before any dose of the IL-2 conjugate is administered to the subject. In some embodiments, one dose of the IL-2 conjugate is administered to the subject before the CAR therapy is administered to the subject, and wherein one or more additional doses of the IL-2 conjugate is administered to the subject after the CAR therapy is administered to the subject. In some embodiments, one dose of the IL-2 conjugate is administered to the subject one day before the CAR therapy is administered to the subject, and one or more additional doses of the IL-2 conjugate is administered to the subject about once every week after the one dose of the IL-2 conjugate is administered to the subject. In any of the above embodiments, after the first dose of the IL-2 conjugate is administered to the subject, the remaining doses of the IL-2 conjugate may be administered to the subject as described in any of the above embodiments, including but not limited to being administered about once every week, about once every two weeks, about once every three weeks, or about once every 4 weeks, or being administered about once every 14, 15, 16, 17, 18, 19, 20, or 21 days, and including but not limited to being administered once, twice, three times, four times, or five times in addition to the first dose, and/or having a total duration of treatment with the IL-2 conjugate (counting from the first dose of the IL-2 conjugate) of 1 month, 2 months, 3 months, 6 months, 9 months, 12 months, 15 months, 18 months, 21 months, or 24 months.

In some instances, the desired doses are conveniently presented in a single dose or as divided doses administered simultaneously (or over a short period of time) or at appropriate intervals, for example as two, three, four or more sub-doses per day.

In some embodiments, a method described herein further comprises administering one or more additional therapeutic agents. In some embodiments, the additional therapeutic agent comprises an antihistamine, such as diphenhydramine. In some embodiments, the additional therapeutic agent comprises a chemotherapeutic agent and an antihistamine, such as diphenhydramine. In some embodiments, the additional therapeutic agent comprises any one of the foregoing chemotherapeutic agents and an antihistamine, such as diphenhydramine.

In some embodiments, the additional therapeutic agent comprises an analgesic, such as acetaminophen. In some embodiments, the additional therapeutic agent comprises a chemotherapeutic agent and an analgesic, such as acetaminophen. In some embodiments, the additional therapeutic agent comprises any one of the foregoing chemotherapeutic agents and an analgesic, such as acetaminophen.

In some embodiments, the additional therapeutic agent comprises one or more vitamins, such as folic acid and/or vitamin B12. In some embodiments, the additional therapeutic agent comprises a chemotherapeutic agent and one or more vitamins, such as folic acid and/or vitamin B12. In some embodiments, the additional therapeutic agent comprises any one of the foregoing chemotherapeutic agents and one or more vitamins, such as folic acid and/or vitamin B12.

In some embodiments, the additional therapeutic agent comprises an antihistamine and an analgesic, such as diphenhydramine and acetaminophen. In some embodiments, the additional therapeutic agent comprises an antihistamine and one or more vitamins, such as diphenhydramine and one or both of folic acid and vitamin B12. In some embodiments, the additional therapeutic agent comprises an analgesic and one or more vitamins, such as acetaminophen and one or both of folic acid and vitamin B12. In some embodiments, the additional therapeutic agent comprises an antihistamine, an analgesic, and one or more vitamins, such as diphenhydramine, acetaminophen, and one or both of folic acid and vitamin B12. In any of the foregoing embodiments, the additional therapeutic agent can further comprise a chemotherapeutic agent, such as any one of the foregoing chemotherapeutic agents.

### Subject

In some embodiments, administration of the IL-2 conjugate is to an adult subject. In some embodiments, the adult subject is a male. In other embodiments, the adult subject is a female. In some embodiments, the adult subject is at least age 12, 15, 18, 19, 20, 25, 30, 35, 40, 45, 50, 55, 60, 65, 70, 75, 80, 85, 90, or 95 years of age. In some embodiments, the adult subject has relapsed or refractory cancer, such as relapsed or refractory DLBCL.

In some embodiments, the subject is ≥12 years of age. In some embodiments, the subject's disease location is amenable to tumor biopsy, e.g., at baseline or prior to initiation of therapy. In some embodiments, the subject has a measurable disease. In some embodiments, the subject, if female, is not pregnant or breastfeeding, is not a woman of childbearing potential (WOCBP) or is a WOCBP who agrees: (1) to use approved contraception method and submit to regular pregnancy testing prior to treatment and for at least 180 days after discontinuing study treatment, and (2) to refrain from donating or cryopreserving eggs for 180 days after discontinuing study treatment. In some embodiments, the subject, if male, agrees to refrain from donating or cryopreserving sperm, and either abstain from heterosexual intercourse or use approved contraception during study treatment and for at least 210 days after discontinuing study treatment. In some embodiments, the subject is capable of giving signed informed consent. In some embodiments, the subject has histologically or cytologically confirmed diagnosis of DLBCL. In some embodiments, the subject has adequate cardiovascular, hematological, liver, and renal function, as determined by a physician. In some embodiments, the subject has been determined (e.g., by a physician) to have a life expectancy greater than or equal to 12 weeks. In some embodiments, the subject has received at least two prior lines of systemic therapy for DLBCL before administration of the first treatment dose of the IL-2 conjugate. In some embodiments, the at least two prior lines of systemic therapy for DLBCL comprises an anthracycline, an anti-CD20 agent, or a combination of an anthracycline and an anti-CD20 agent. In some embodiments, the anti-CD20 agent comprises rituximab. In some embodiments, the at least two prior lines of systemic therapy for DLBCL comprises a CAR therapy, including any one of the CAR therapies disclosed herein. In some embodiments, the CAR therapy is the last line of the at least two prior lines of systemic therapy for DLBCL. In some embodiments, the subject meets each of the foregoing criteria.

In some embodiments, the subject does not have Eastern Cooperative Oncology Group (ECOG) performance status of >2 (for a subject ≥16 years old). In some embodiments, the subject does not have a Lansky Scale (for a subject <16 years old)<50%. In some embodiments, the subject does not have poor bone marrow reserve. In some embodiments, the subject does not have poor organ function. In some embodiments, the subject does not have baseline SpO2≤92%. In some embodiments, the subject does not have lymphomatous involvement of the central nervous system. In some embodiments, the subject does not have a history of allogenic or solid organ transplant. In some embodiments, the subject did not receive a final administration of prior antitumor therapy or any investigational treatment within 21 days or less than 5 times the half-life, whichever is shorter, of receiving the IL-2 conjugate. In some embodiments, the subject did not have major surgery or local intervention within 21 days of receiving the IL-2 conjugate. In some embodiments, the subject did not receive prior IL-2-based anticancer treatment. In some embodiments, the subject does not have a comorbidity requiring corticosteroid therapy. In some embodiments, the subject did not use an antibiotic (other than topical antibiotics)≤14 days prior to first dose of IL-2 conjugate. In some embodiments, the subject did not have a severe or unstable cardiac condition within 6 months prior to starting study treatment. In some embodiments, the subject does not have active, known, or suspected autoimmune disease that has required systemic treatment in the past 2 years. In some embodiments, the subject does not have a known second malignancy either progressing or requiring active treatment within the last 3 years. In some embodiments, the subject did not receive a live or live attenuated virus vaccination (except seasonal flu vaccines or SARS-CoV-2 vaccines that do not contain live virus) within 28 days of planned treatment start. In some embodiments, the subject does not have any of the characteristics listed in this paragraph. In some embodiments, the subject does not have any of the characteristics listed in this paragraph and meets each of the criteria in the preceding paragraph.

In some embodiments, the subject has no known hypersensitivity or contraindications to the IL-2 conjugate being administered or to PEG. In some embodiments, the subject has no known hypersensitivity or contraindications to the IL-2 conjugate being administered, PEG, or pegylated drugs.

In some embodiments, the subject does not have any serious medical condition (including pre-existing autoimmune disease or inflammatory disorder), laboratory abnormality, psychiatric condition, or any other significant or unstable concurrent medical illness that would preclude treatment or would make treatment inappropriate.

In some embodiments, the subject is not pregnant or breastfeeding. In some embodiments, the subject is not expecting to conceive or father children during the course of the treatment and following up to 1, 2, 3, 4, 5, 6, or 7 months after administration of the final treatment dose.

In some embodiments, the subject is not receiving a concurrent therapy with any investigational agent, vaccine, or device during the course of treatment. In some embodiments, the subject is receiving concurrent therapy with an investigational agent, vaccine, or device during the course of treatment after physician approval.

### Effects of Administration

In some embodiments, administration of the IL-2 conjugate provides a complete response, a partial response, or stable disease.

In some embodiments, following administration of the IL-2 conjugate, the subject experiences a response as measured by the Lugano response criteria 2014. In some embodiments, following administration of the IL-2 conjugate, the subject experiences an Objective Response Rate (ORR) according to the Lugano response criteria 2014. In some embodiments, following administration of the IL-2 conjugate, the subject experiences a Duration of Response (DOR) according to the Lugano response criteria 2014. In some embodiments, following administration of the IL-2 conjugate, the subject experiences Progression-Free Survival (PFS) according to the Lugano response criteria 2014. In some embodiments, following administration of the IL-2 conjugate, the subject experiences Overall Survival according to the Lugano response criteria 2014. In some embodiments, following administration of the IL-2 conjugate, the subject experiences a Time to Response (TTR) according to the Lugano response criteria 2014. In some embodiments, following administration of the IL-2 conjugate, the subject experiences a Clinical Benefit Rate (CBR) according to the Lugano response criteria 2014.

In some embodiments, administration of the IL-2 conjugate to the subject does not cause vascular leak syndrome in the subject. In some embodiments, administration of the IL-2 conjugate to the subject does not cause Grade 2, Grade 3, or Grade 4 vascular leak syndrome in the subject. In some embodiments, administration of the IL-2 conjugate to the subject does not cause Grade 2 vascular leak syndrome in the subject. In some embodiments, administration of the IL-2 conjugate to the subject does not cause Grade 3 vascular leak syndrome in the subject. In some embodiments, administration of the IL-2 conjugate to the subject does not cause Grade 4 vascular leak syndrome in the subject. In some embodiments, administration of the IL-2 conjugate to the subject does not cause loss of vascular tone in the subject.

In some embodiments, administration of the IL-2 conjugate to the subject does not cause extravasation of plasma proteins and fluid into the extravascular space in the subject.

In some embodiments, administration of the IL-2 conjugate to the subject does not cause hypotension and reduced organ perfusion in the subject.

In some embodiments, administration of the IL-2 conjugate to the subject does not cause impaired neutrophil function in the subject. In some embodiments, administration of the IL-2 conjugate to the subject does not cause reduced chemotaxis in the subject.

In some embodiments, administration of the IL-2 conjugate to the subject is not associated with an increased risk of disseminated infection in the subject. In some embodiments, the disseminated infection is sepsis or bacterial endocarditis. In some embodiments, the disseminated infection is sepsis. In some embodiments, the disseminated infection is bacterial endocarditis. In some embodiments, the subject is treated for any preexisting bacterial infections prior to administration of the IL-2 conjugate. In some embodiments, the subject is treated with an antibacterial agent selected from oxacillin, nafcillin, ciprofloxacin, and vancomycin prior to administration of the IL-2 conjugate.

In some embodiments, administration of the IL-2 conjugate to the subject does not exacerbate a pre-existing or initial presentation of an autoimmune disease or an inflammatory disorder in the subject. In some embodiments, the administration of the IL-2 conjugate to the subject does not exacerbate a pre-existing or initial presentation of an autoimmune disease in the subject. In some embodiments, the administration of the IL-2 conjugate to the subject does not exacerbate a pre-existing or initial presentation of an inflammatory disorder in the subject. In some embodiments, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments, the autoimmune disease or inflammatory disorder in the subject is Crohn's disease. In some embodiments, the autoimmune disease or inflammatory disorder in the subject is scleroderma. In some embodiments, the autoimmune disease or inflammatory disorder in the subject is thyroiditis. In some embodiments, the autoimmune disease or inflammatory disorder in the subject is inflammatory arthritis. In some embodiments, the autoimmune disease or inflammatory disorder in the subject is diabetes mellitus. In some embodiments, the autoimmune disease or inflammatory disorder in the subject is oculo-bulbar myasthenia gravis. In some embodiments, the autoimmune disease or inflammatory disorder in the subject is crescentic IgA glomerulonephritis. In some embodiments, the autoimmune disease or inflammatory disorder in the subject is cholecystitis. In some embodiments, the autoimmune disease or inflammatory disorder in the subject is cerebral vasculitis. In some embodiments, the autoimmune disease or inflammatory disorder in the subject is Stevens-Johnson syndrome. In some embodiments, the autoimmune disease or inflammatory disorder in the subject is bullous pemphigoid.

In some embodiments, administration of the IL-2 conjugate to the subject does not cause changes in mental status, speech difficulties, cortical blindness, limb or gait ataxia, hallucinations, agitation, obtundation, or coma in the subject. In some embodiments, administration of the IL-2 conjugate to the subject does not cause seizures in the subject. In some embodiments, administration of the IL-2 conjugate to the subject is not contraindicated in subjects having a known seizure disorder.

In some embodiments, administration of the IL-2 conjugate to the subject does not cause capillary leak syndrome in the subject. In some embodiments, administration of the IL-2 conjugate to the subject does not cause Grade 2, Grade 3, or Grade 4 capillary leak syndrome in the subject. In some embodiments, administration of the IL-2 conjugate to the subject does not cause Grade 2 capillary leak syndrome in the subject. In some embodiments, administration of the IL-2 conjugate to the subject does not cause Grade 3 capillary leak syndrome in the subject. In some embodiments, administration of the IL-2 conjugate to the subject does not cause Grade 4 capillary leak syndrome in the subject.

In some embodiments, administration of the IL-2 conjugate to the subject does not cause a drop in mean arterial blood pressure in the subject following administration. In some embodiments, administration of the IL-2 conjugate to the subject does cause hypotension in the subject. In some embodiments, administration of the IL-2 conjugate to the subject does not cause the subject to experience a systolic blood pressure below 90 mm Hg or a 20 mm Hg drop from baseline systolic pressure.

In some embodiments, administration of the IL-2 conjugate to the subject does not cause edema or impairment of kidney or liver function in the subject.

In some embodiments, administration of the IL-2 conjugate to the subject does not cause eosinophilia in the subject. In some embodiments, administration of the IL-2 conjugate to the subject does not cause the eosinophil count in the peripheral blood of the subject to exceed 500 per μL. In some embodiments, administration of the IL-2 conjugate to the subject does not cause the eosinophil count in the peripheral blood of the subject to exceed 500 μL to 1,500 per μL. In some embodiments, administration of the the IL-2 conjugate to the subject does not cause the eosinophil count in the peripheral blood of the subject to exceed 1,500 per μL to 5,000 per μL. In some embodiments, administration of the IL-2 conjugate to the subject does not cause the eosinophil count in the peripheral blood of the subject to exceed 5,000 per μL. In some embodiments, administration of the IL-2 conjugate to the subject is not contraindicated in subjects on an existing regimen of psychotropic drugs.

In some embodiments, administration of the IL-2 conjugate to the subject is not contraindicated in subjects on an existing regimen of nephrotoxic, myelotoxic, cardiotoxic, or hepatotoxic drugs. In some embodiments, administration of the IL-2 conjugate to the subject is not contraindicated in subjects on an existing regimen of aminoglycosides, cytotoxic chemotherapy, doxorubicin, methotrexate, or asparaginase. In some embodiments, administration of the IL-2 conjugate to the subject is not contraindicated in subjects receiving combination regimens containing antineoplastic agents. In some embodiments, the antineoplastic agent is selected from dacarbazine, cis-platinum, tamoxifen, and interferon-alpha.

In some embodiments, administration of the IL-2 conjugate to the subject does not cause one or more Grade 4 adverse events in the subject following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments, administration of the IL-2 conjugate to a group of subjects does not cause one or more Grade 4 adverse events in greater than 1% of the subjects following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments, administration of the IL-2 conjugate to a group of subjects does not cause one or more adverse events in greater than 1% of the subjects following administration, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments, administration of the IL-2 conjugate to a group of subjects does not cause one or more adverse events in greater than 1% of the subjects following administration, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments, administration of the IL-2 conjugate to the subject stimulates CD8+ cells in a subject. In some embodiments, administration of the IL-2 conjugate to the subject stimulates NK cells in a subject. Stimulation may comprise an increase in the number of CD8+ cells in the subject, e.g., about 4, 5, 6, or 7 days after administration, or about 1, 2, 3, or 4 weeks after administration. In some embodiments, the CD8+ cells comprise memory CD8+ cells. In some embodiments, the CD8+ cells comprise effector CD8+ cells. Stimulation may comprise an increase in the proportion of CD8+ cells that are Ki67 positive in the subject, e.g., about 4, 5, 6, or 7 days after administration, or about 1, 2, 3, or 4 weeks after administration. Stimulation may comprise an increase in the number of NK cells in the subject, e.g., about 4, 5, 6, or 7 days after administration, or about 1, 2, 3, or 4 weeks after administration.

In some embodiments, CD8+ cells are expanded in the subject following administration of the IL-2 conjugate by at least 1.5-fold, such as by at least 1.6-fold, 1.7-fold, 1.8-fold, 1.9-fold, 2.0-fold, 2.1-fold, 2.2-fold, 2.3-fold, 2.4-fold, or 2.5-fold. In some embodiments, NK cells are expanded in the subject following administration of the IL-2 conjugate by at least 3.5-fold, such as by at least 4.0-fold, 4.5-fold, 5.5-fold, 6-fold, 6.5-fold, 7.0-fold, or 7.5-fold. In some embodiments, eosinophils are expanded in the subject following administration of the IL-2 conjugate by no more than about 2-fold, such as no more than about 1.5-fold, 1.4-fold, or 1.3-fold. In some embodiments, CD4+ cells are expanded in the subject following administration of the IL-2 conjugate by no more than about 2-fold, such as no more than about 1.8-fold, 1.7-fold, or 1.6-fold. In some embodiments, the expansion of CD8+ cells and/or NK cells in the subject following administration of the IL-2 conjugate is greater than the expansion of CD4+ cells and/or eosinophils. In some embodiments, the expansion of CD8+ cells is greater than the expansion of CD4+ cells. In some embodiments, the expansion of NK cells is greater than the expansion of CD4+ cells. In some embodiments, the expansion of CD8+ cells is greater than the expansion of eosinophils. In some embodiments, the expansion of NK cells is greater than the expansion of eosinophils. Fold expansion is determined relative to a baseline value measured before administration of the IL-2 conjugate. In some embodiments, fold expansion is determined at any of the times after administration, such as about 4, 5, 6, or 7 days after administration, or about 1, 2, 3, or 4 weeks after administration.

In some embodiments, administration of the IL-2 conjugate to the subject increases the number of peripheral CD8+ T and NK cells in the subject without increasing the number of peripheral CD4+ regulatory T cells in the subject. In some embodiments, administration of the IL-2 conjugate to the subject increases the number of peripheral CD8+ T and NK cells in the subject without increasing the number of peripheral eosinophils in the subject. In some embodiments, administration of the IL-2 conjugate to the subject increases the number of peripheral CD8+ T and NK cells in the subject without increasing the number of intratumoral CD8+ T and NK cells in the subject and without increasing the number of intratumoral CD4+ regulatory T cells in the subject.

In some embodiments, administration of the IL-2 conjugate to the subject does not require the availability of an intensive care facility or skilled specialists in cardiopulmonary or intensive care medicine. In some embodiments, administration of the IL-2 conjugate to the subject does not require the availability of an intensive care facility or skilled specialists in cardiopulmonary or intensive care medicine. In some embodiments, administration of the IL-2 conjugate to the subject does not require the availability of an intensive care facility. In some embodiments, administration of the IL-2 conjugate to the subject does not require the availability of skilled specialists in cardiopulmonary or intensive care medicine.

In some embodiments, administration of the IL-2 conjugate does not cause dose-limiting toxicity. In some embodiments, administration of the IL-2 conjugate does not cause severe cytokine release syndrome. In some embodiments, the IL-2 conjugate does not induce anti-drug antibodies (ADAs), i.e., antibodies against the IL-2 conjugate. In some embodiments, a lack of induction of ADAs is determined by direct immunoassay for antibodies against PEG and/or ELISA for antibodies against the IL-2 conjugate. An IL-2 conjugate is considered not to induce ADAs if a measured level of ADAs is statistically indistinguishable from a baseline (pre-treatment) level or from a level in an untreated control.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Preparation of Pegylated IL-2 Conjugates

An exemplary method with details for preparing IL-2 conjugates described herein is provided in this Example.

IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which an unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) N6-((2-azidoethoxy)-carbonyl)-L-lysine (AzK); and (d) a truncated variant of nucleotide triphosphate transporter PtNTT2 in which the first 65 amino acid residues of the full-length protein were deleted. The double-stranded oligonucleotide that encodes the amino acid sequence of the desired IL-2 variant contained a codon AXC as codon 64 of the sequence that encodes the protein having SEQ ID NO: 1 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties to the AzK. Exemplary reactions are shown in Schemes 1 and 2 (wherein n indicates the number of repeating PEG units). The reaction of the AzK moiety with the DBCO alkynyl moiety may afford one regioisomeric product or a mixture of regioisomeric products.

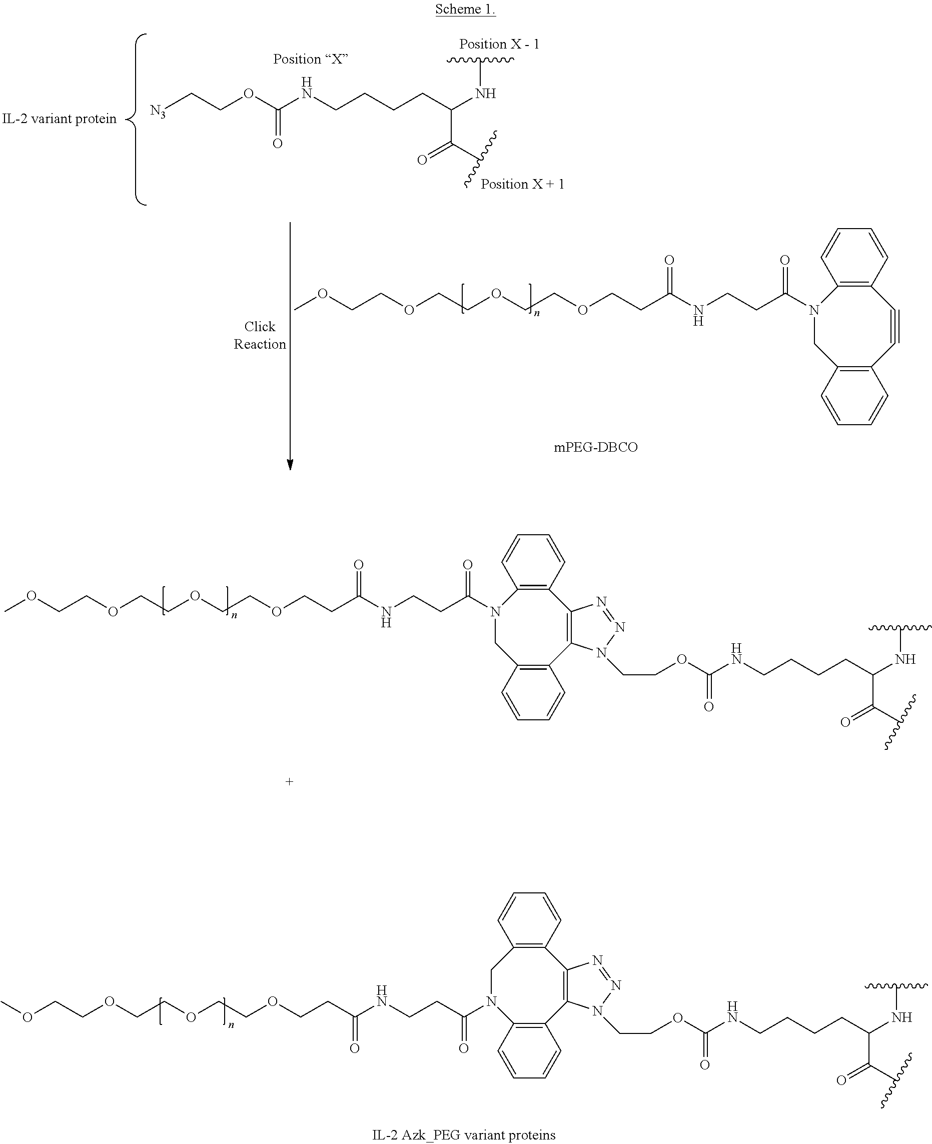

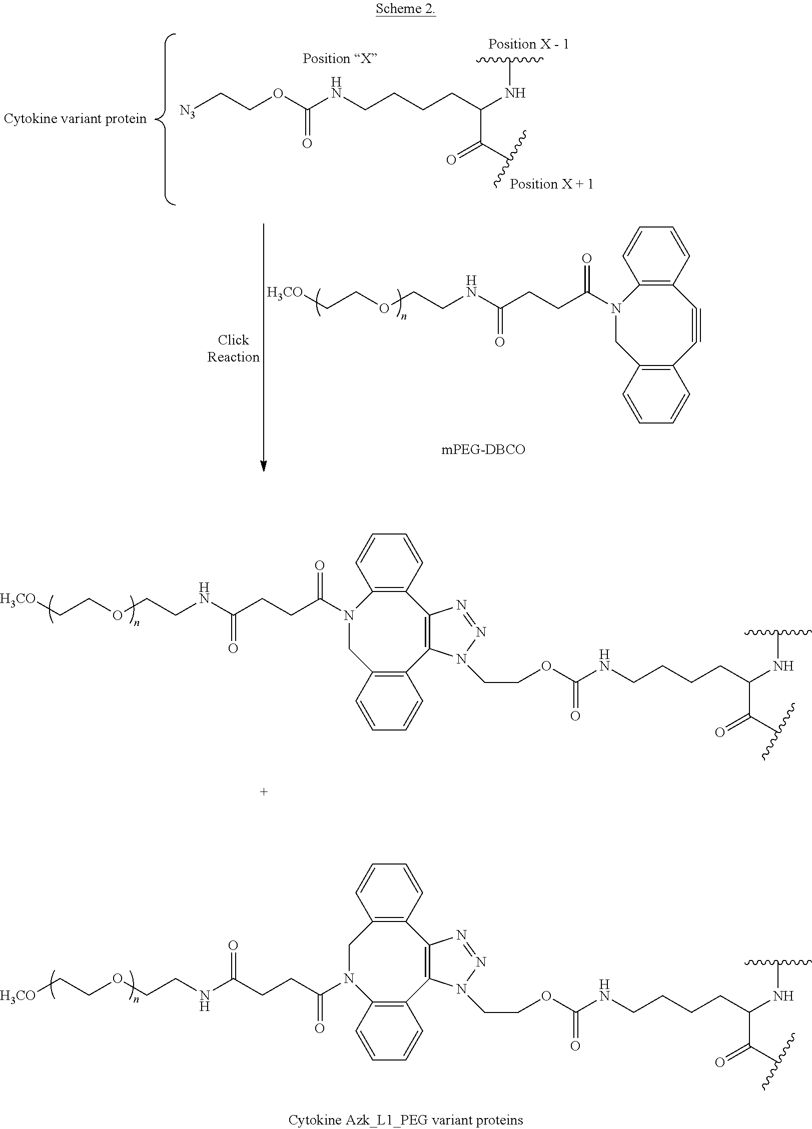

### Example 2. Clinical Study of Biomarker Effects Following IL-2 Conjugate Administration (8 μg/kg, 16 μg/kg, and 24 μg/kg [Q3W])

Cohort Using 24 μg/kg Dose [Q3W]

A study was performed to characterize immunological effects of in vivo administration of an IL-2 conjugate described herein. The IL-2 conjugate comprised SEQ ID NO: 2, wherein position 64 is AzK_L1_PEG30 kD, where AzK_L1_PEG30 kD is defined as a structure of Formula (IV) or Formula (V), or a mixture of Formula (IV) and Formula (V), and a 30 kDa, linear mPEG chain. This IL-2 conjugate can also be described as an IL-2 conjugate comprising SEQ ID NO: 1, wherein position 64 is replaced by the structure of Formula (IV) or Formula (V), or a mixture of Formula (IV) and Formula (V), and a 30 kDa, linear mPEG chain. The IL-2 conjugate can also be described as an IL-2 conjugate comprising SEQ ID NO: 1, wherein position 64 is replaced by the structure of Formula (XII) or Formula (XIII), or a mixture of Formula (XII) and Formula (XIII), and a 30 kDa, linear mPEG chain. This IL-2 conjugate has a proposed International Nonproprietary Name (pINN) of pegenzileukin. The compound was prepared using methods wherein a protein was first prepared having SEQ ID NO: 1 in which the proline at position 64 was replaced by N6-((2-azidoethoxy)-carbonyl)-L-lysine AzK. The AzK-containing protein was then allowed to react under click chemistry conditions with DBCO comprising a methoxy, linear PEG group having an average molecular weight of 30 kDa, followed by purification and formulation employing standard procedures. Here and throughout the examples, drug mass per kg subject (e.g., 24 μg/kg) refers to IL-2 mass exclusive of PEG and linker mass.

The IL-2 conjugate was administered via IV infusion at a dose of 24 μg/kg for 30 minutes every 3 weeks [Q3W]. Effects on the following biomarkers were analyzed as surrogate predictors of safety and/or efficacy:
Eosinophilia (elevated peripheral eosinophil count): Cell surrogate marker for IL-2-induced proliferation of cells (eosinophils) linked to vascular leak syndrome (VLS);
Interleukin 5 (IL-5): Cytokine surrogate marker for IL-2 induced activation of type 2 innate lymphoid cells and release of this chemoattractant that leads to eosinophilia and potentially VLS;
Interleukin 6 (IL-6): Cytokine surrogate marker for IL-2 induced cytokine release syndrome (CRS); and
Interferon γ (IFN-γ): Cytokine surrogate marker for IL-2 induced activation of CD8+ cytotoxic T lymphocytes.

Effects on the following biomarkers were analyzed as surrogate predictors of anti-tumor immune activity:
Peripheral CD8+ Effector Cells: Marker for IL-2-induced proliferation of these target cells in the periphery that upon infiltration become a surrogate marker of inducing a potentially latent therapeutic response;
Peripheral CD8+ Memory Cells: Marker for IL-2-induced proliferation of these target cells in the periphery that upon infiltration become a surrogate marker of inducing a potentially durable latent therapeutic and maintenance of the memory population;
Peripheral NK Cells: Marker for IL-2-induced proliferation of these target cells in the periphery that upon infiltration become a surrogate marker of inducing a potentially rapid therapeutic response; and
Peripheral CD4+ Regulatory Cells: Marker for IL-2-induced proliferation of these target cells in the periphery that upon infiltration become a surrogate marker of inducing an immunosuppressive TME and offsetting of an effector-based therapeutic effect.

Subjects were human males or females aged ≥18 years at screening. All subjects had been previously treated with an anti-cancer therapy and met at least one of the following: Treatment related toxicity resolved to grade 0 or 1 (alopecia excepted) according to NCI CTCAE v5.0; or Treatment related toxicity resolved to at least grade 2 according to NCI CTCAE v5.0 with prior approval of the Medical Monitor. The most common tumors were colorectal or melanoma.

Subjects also met the following criteria: Provided informed consent. Eastern Cooperative Oncology Group (ECOG) performance status of 0 or 1. Life expectancy greater than or equal to 12 weeks as determined by the Investigator. Histologically or cytologically confirmed diagnosis of advanced and/or metastatic solid tumors. Subjects with advanced or metastatic solid tumors who have refused standard of care; or for whom no reasonable standard of care exists that would confer clinical benefit; or for whom standard therapy is intolerable, not effective, or not accessible. Measurable disease per RECIST v1.1. Adequate laboratory parameters including: Absolute lymphocyte count ≥0.5 times lower limit of normal; Platelet count ≥100×10⁹/L; Hemoglobin ≥9.0 g/dL (absence of growth factors or transfusions within 2 weeks; 1-week washout for ESA and CSF administration is sufficient); Absolute neutrophil count ≥1.5×10⁹/L (absence of growth factors within 2 weeks); Prothrombin time (PT) and partial thromboplastin time (PTT)≤1.5 times upper limit of normal (ULN); Aspartate aminotransferase (AST) and alanine aminotransferase (ALT)≤2.5 times ULN except if liver metastases are present may be ≤5 times ULN; Total bilirubin ≤1.5×ULN. Premenopausal women and women less than 12 months after menopause had a negative serum pregnancy test within 7 days prior to initiating study treatment.

Q3W dosing. 11 individuals (4 [36.4%] male, 7 [63.6%] female, [9 [81.8%] Caucasian) having advanced or metastatic solid tumors and whose median age was 67, ranging from (37-78), received the IL-2 conjugate at a 24 μg/kg dose Q3W for up to nine cycles (1 dose per cycle). Here and throughout discussion of the first cohort of Example 2, drug mass per kg subject (e.g., 24 μg/kg) refers to IL-2 mass exclusive of PEG and linker mass.

One subject had partial response at initial scan confirmed on second and third scan (prior PD-1 exposure) ongoing for 6+ months; five subjects had initial disease stabilization (at the 6-week assessment), three subjects had progressive disease at first assessment, and one subject came off treatment for an adverse event. All subjects had peak post-dose CD8+ Ki67 expression levels that exceeded 50 percent (50%-85%).

One 73-year-old male subject with squamous cell carcinoma of unknown origin who received 7 cycles of treatment (24 μg/kg Q3W) and who had also received two lines of systemic therapy including anti-PD-1 (best response on anti-PD-1: SD) showed tumor reduction of 31% after two cycles. The maximal tumor responses in other patients with immune sensitive tumors were found to be renal cell carcinoma (RCC) (16% growth) and Melanoma (10% growth observed in two subjects; 2% reduction; and 20% reduction).

The peripheral expansion of CD8+ T effector cells averaged 4.47-fold above baseline. All subjects had elevated post-dose NK Cell Ki67 expression levels. The subjects had peak post-dose peripheral expansion of NK cells that averaged 7.67-fold above baseline.

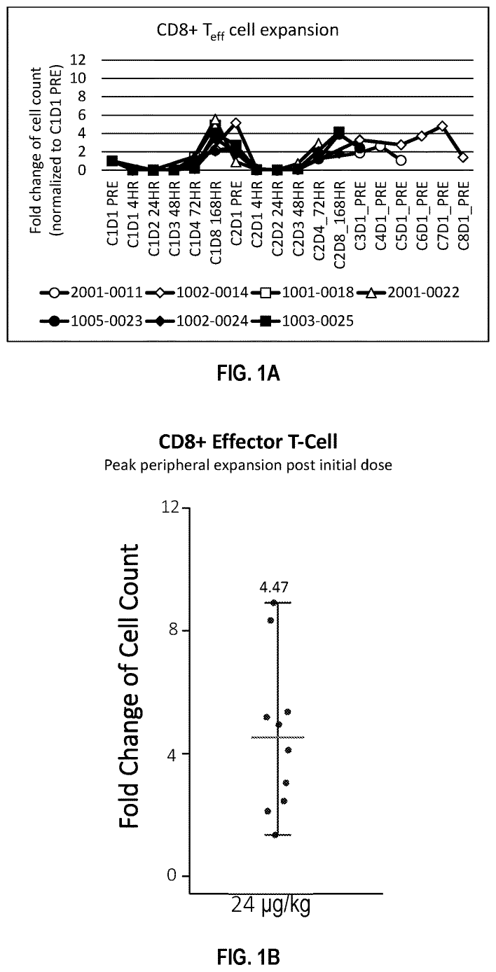

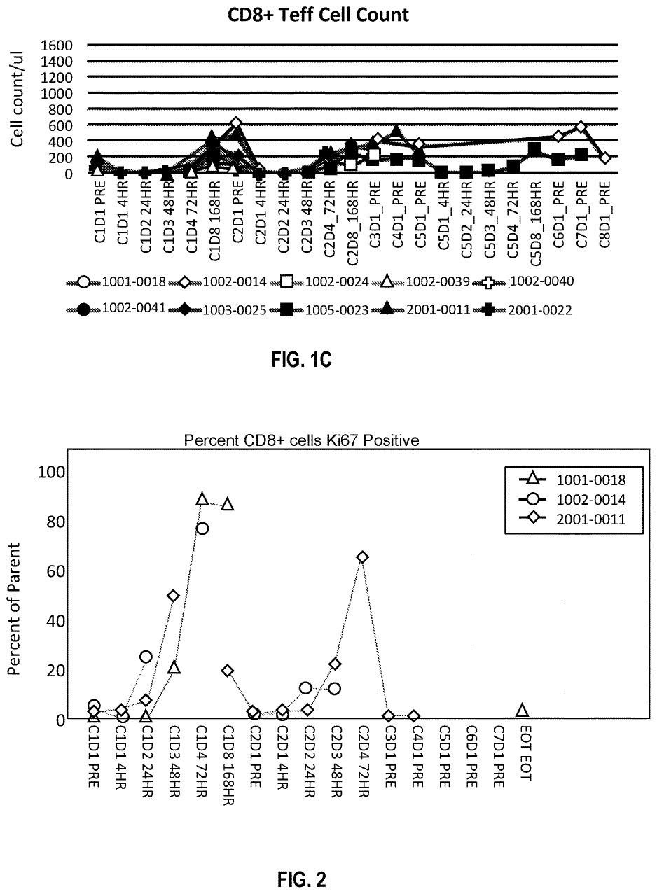

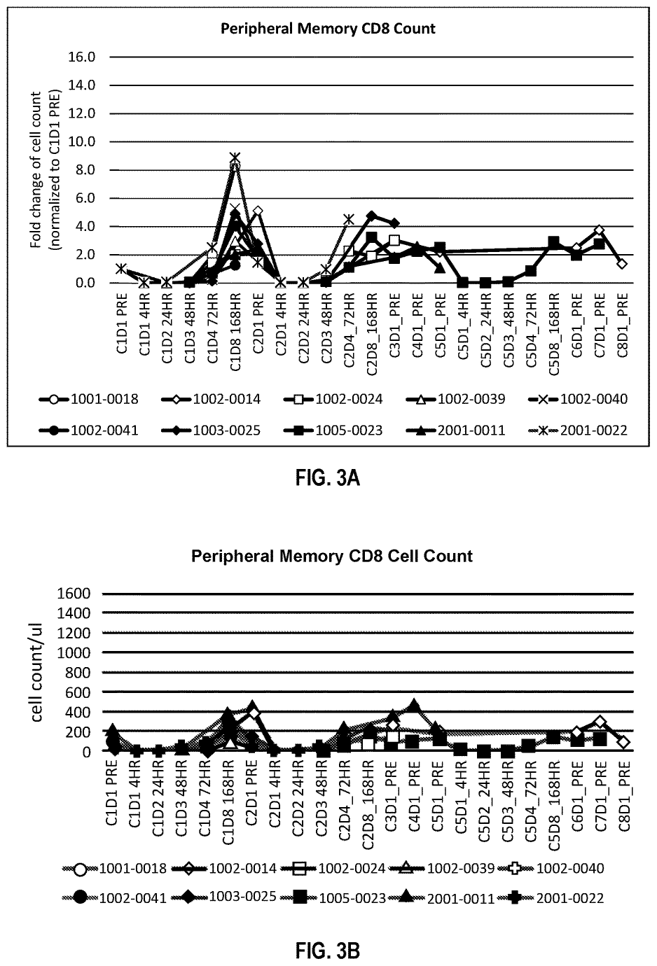

Efficacy biomarkers. Data for efficacy biomarkers was based on data available for up to 10 subjects receiving the IL-2 conjugate at 24 μg/kg. Peripheral CD8+ T_{eff} cell counts were measured (FIGS. 1A-C). Prolonged CD8+ expansion over baseline (e.g., greater than or equal to 2-fold change) was observed at 3 weeks after the previous dose in some subjects. The percentage of CD8+ T_{eff} cells expressing Ki67 was also measured (FIG. 2). Peripheral CD8+ memory cells counts are shown in FIGS. 3A-3B.

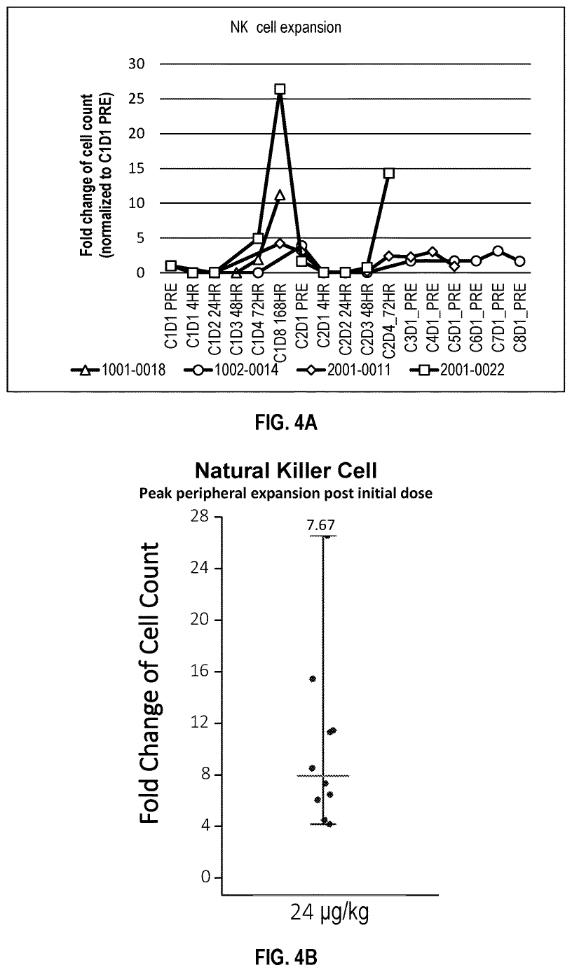

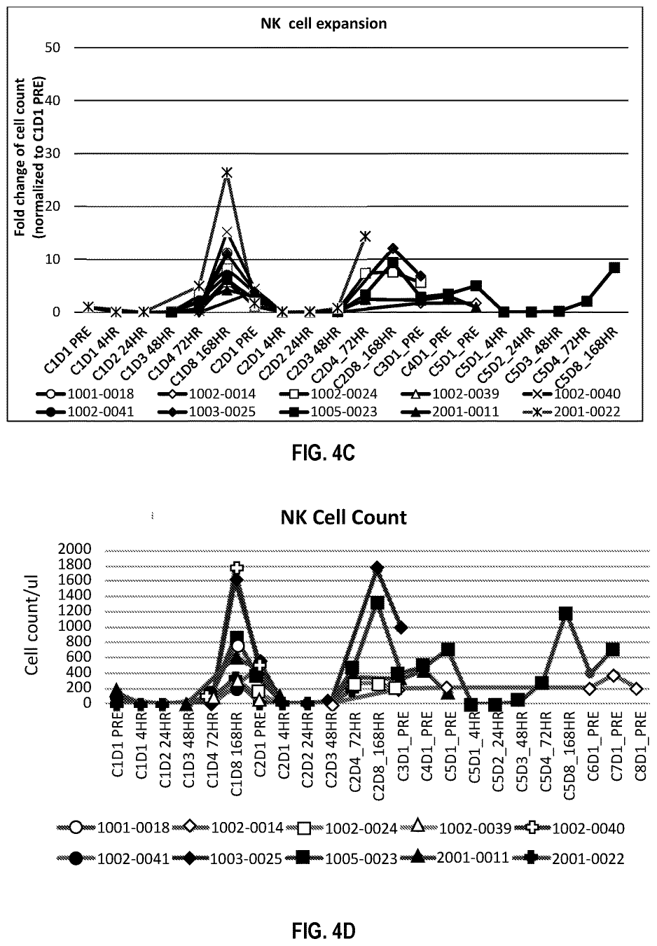

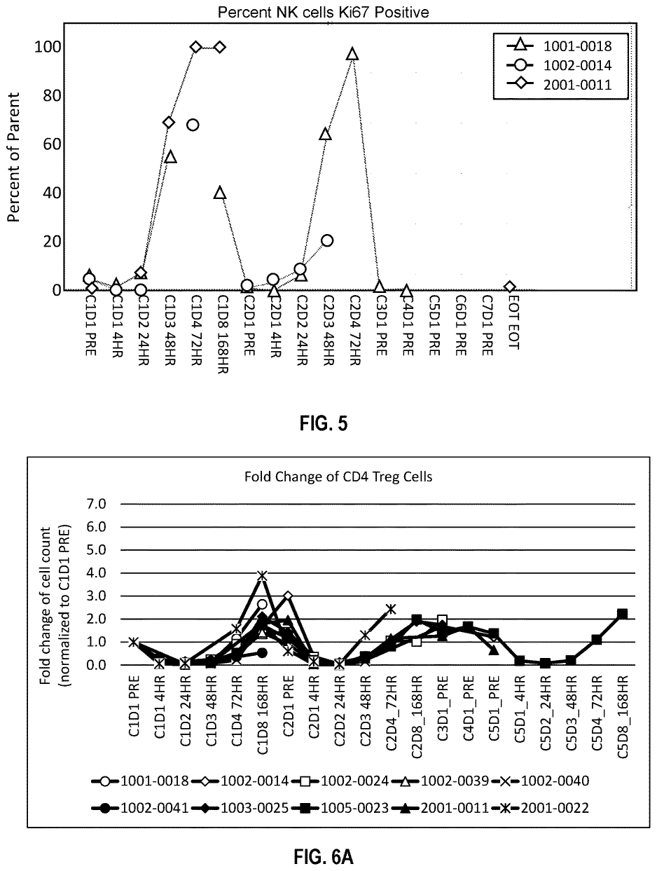

Peripheral NK cell counts are shown in FIGS. 4A-4D. An increase in NK cell count was observed in each subject. The percentage of NK cells expressing Ki67 was also measured (FIG. 5).

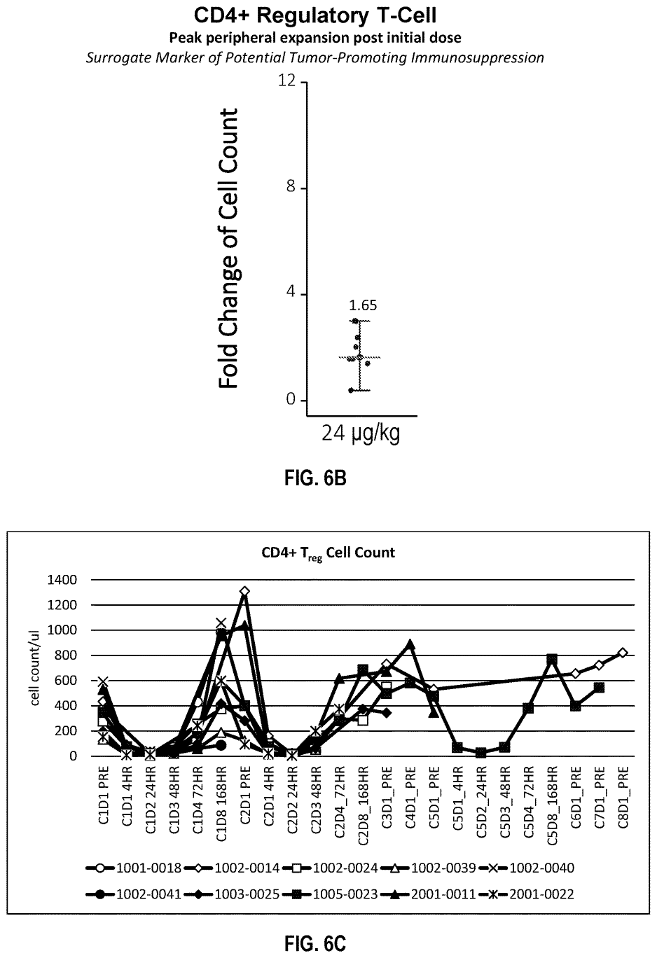

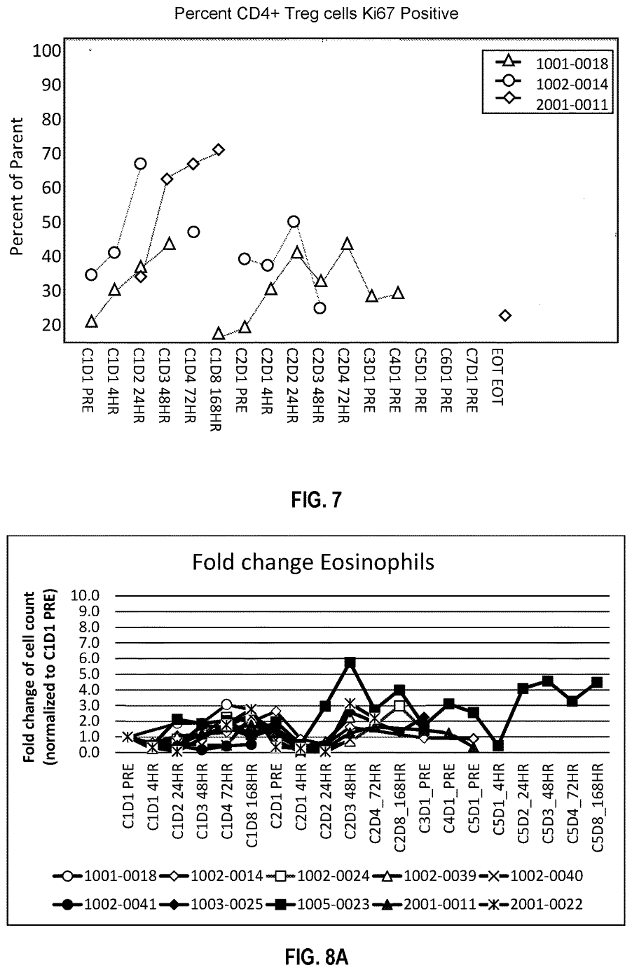

Peripheral CD4+ T_{reg} counts are shown in FIGS. 6A-6C. The percentage of CD4+ T_{reg} cells expressing Ki67 was also measured (FIG. 7).

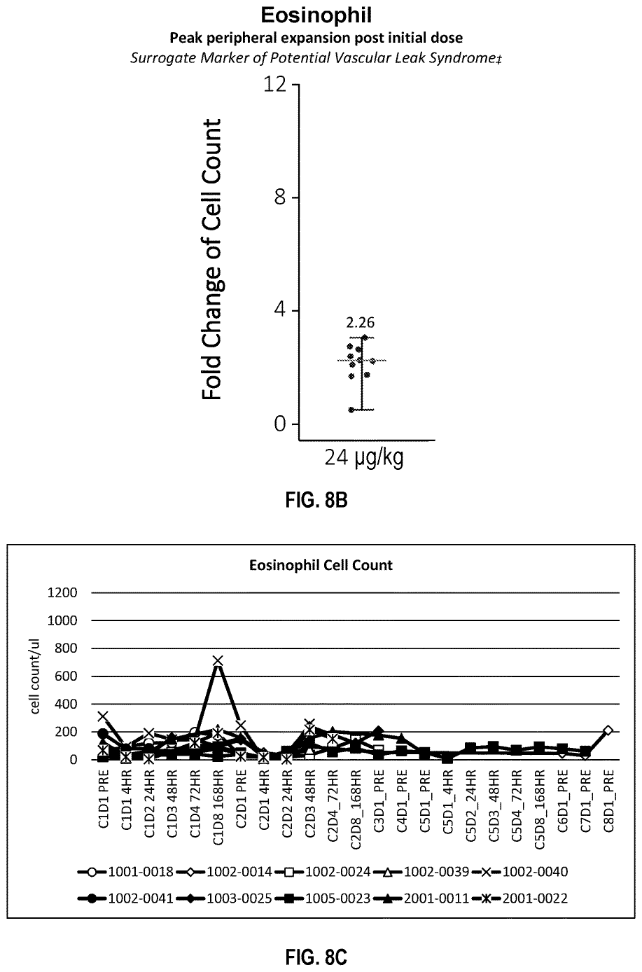

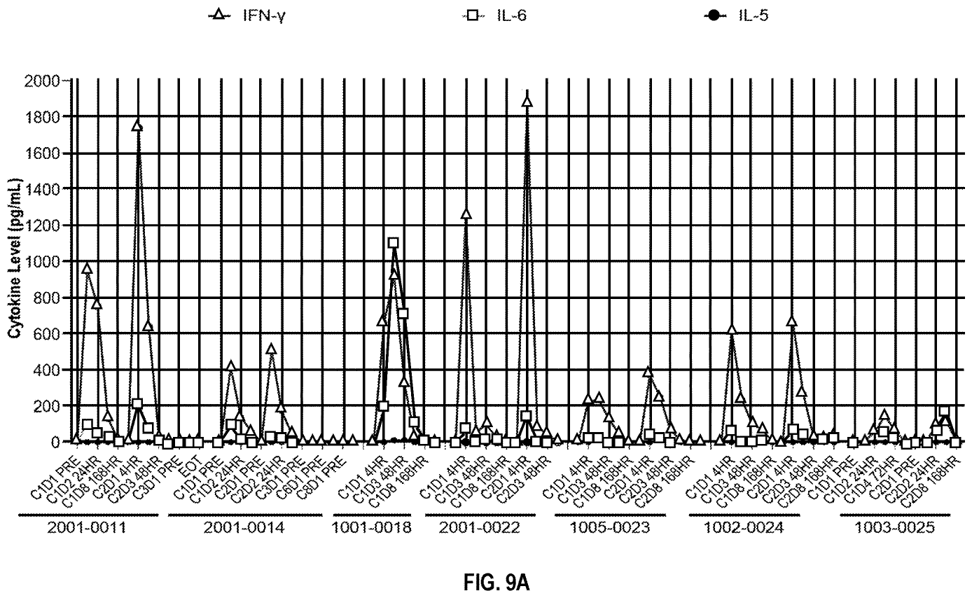

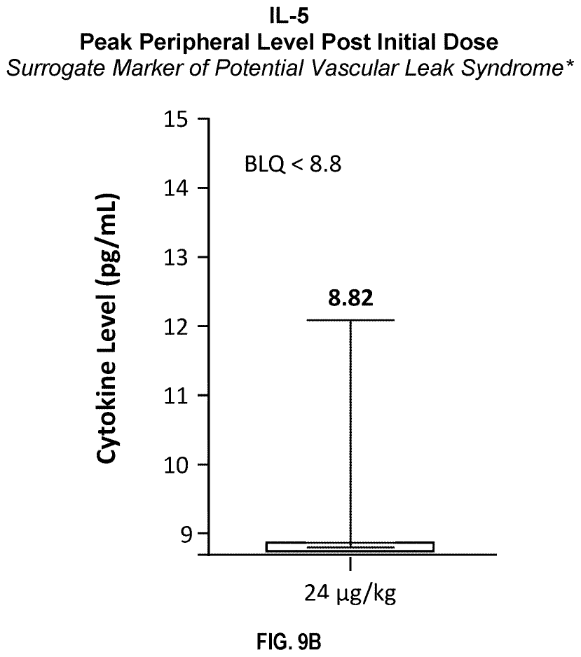

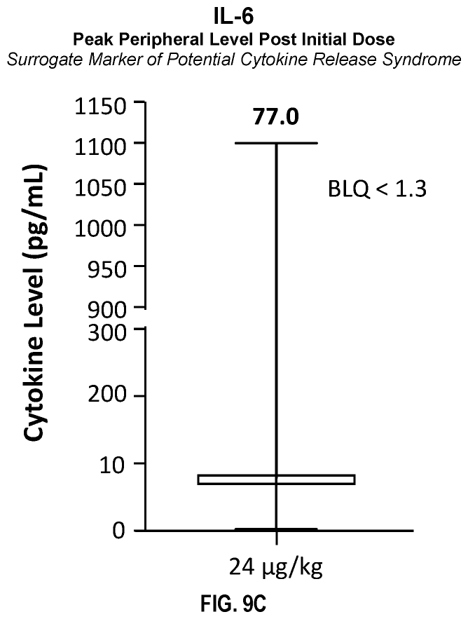

Eosinophil counts were measured (FIGS. 8A-8C). The measured values did not exceed a four-fold increase and were consistently below the range of 2,328-15,958 eosinophils/μL in patients with IL-2 induced eosinophilia as reported in Pisani et al., Blood 1991 Sep. 15; 78 (6): 1538-44. Levels of IFN-γ, IL-5, and IL-6 were also measured (FIGS. 9A-9C). The measured values show that IFN-γ was induced, but low amounts of IL-5 and IL-6, cytokines associated with VLS and CRS, respectively, were induced, except for one subject in whom IL-6 levels increased to about 1,100 μg/mL at 24 hours after treatment (after receiving tocilizumab) but decreased thereafter.

Anti-drug Antibodies (ADAs). Samples from treated subjects were assayed after each dose cycle for anti-drug antibodies (ADAs). Anti-polyethylene glycol autoantibodies were detected by direct immunoassays (detection limit: 36 ng/mL). A bridging MesoScale Discovery ELISA was performed with a labeled form of the IL-2 conjugate, having a detection limit of 4.66 ng/mL. Additionally, a cell-based assay for neutralizing antibodies against the IL-2 conjugate was performed using the CTLL-2 cell line, with STAT5 phosphorylation as the readout (detection limit: 6.3 μg/mL).

Samples were collected and analyzed after each dose cycle from two subjects who received 5 dose cycles and one subject who received 4 dose cycles. An assay-specific cut point was determined during assay qualification as a signal to negative ratio of 1.09 or higher for the IL-2 conjugate ADA assay and 2.08 for the PEG ADA assay. Samples that gave positive or inconclusive results in the IL-2 conjugate assay were subjected to confirmatory testing in which samples and controls were assayed in the presence and absence of confirmatory buffer (10 μg/mL IL-2 conjugate in blocking solution). Samples that gave positive or inconclusive results in the PEG assay were subjected to confirmatory testing in which samples and controls were assayed in the presence and absence of confirmatory buffer (10 μg/mL IL-2 conjugate in 6% horse serum). Samples will be considered “confirmed” if their absorbance signal is inhibited by equal to or greater than an assay-specific cut point determined during assay qualification (14.5% for the IL-2 conjugate or 42.4% for PEG) in the detection step. No confirmed ADA against the IL-2 conjugate or PEG were detected (data not shown).

Summary of Results; Discussion. All subjects tested had post-dose CD8+ Ki67 expression levels exceeding 50% (50%-85%) at one or more time points and peripheral expansion of CD8+ T effector (Teff) cells. All subjects tested also had post-dose NK cell Ki67 expression levels exceeding 50% (50%-100%) at one or more time points with peripheral expansion of NK cells. There were no meaningful elevations in IL-5 levels and the subject whose IL-6 level was increased at day 3 showed a reduction the following day. No ADAs were induced in any of the tested subjects.

An AE was any untoward medical occurrence in a clinical investigation subject administered a pharmaceutical product, regardless of causal attribution. Dose-limiting toxicities were defined as an AE occurring within Day 1 through Day 29 (inclusive)+1 day of a treatment cycle that was not clearly or incontrovertibly solely related to an extraneous cause and that met at least one of the following criteria:
Grade 3 neutropenia (absolute neutrophil count <1,000/mm³>500/mm³) lasting ≥7 days, or Grade 4 neutropenia of any duration
Grade 3+ febrile neutropenia
Grade 4+ thrombocytopenia (platelet count <25,000/mm³)
Grade 3+ thrombocytopenia (platelet count <50,000-25,000/mm³) lasting ≥5 days, or associated with clinically significant bleeding or requiring platelet transfusion
Failure to meet recovery criteria of an absolute neutrophil count of at least 1,000 cells/mm³ and a platelet count of at least 75,000 cells/mm³ within 10 days
Any other grade 4+ hematologic toxicity lasting ≥5 days
Grade 3+ ALT or AST in combination with a bilirubin >2 times ULN with no evidence of cholestasis or another cause such as viral infection or other drugs (i.e., Hy's law)
Grade 3 infusion-related reaction that occurs with premedication; Grade 4 infusion-related reaction
Grade 3 Vascular Leak Syndrome defined as hypotension associated with fluid retention and pulmonary edema
Grade 3+ anaphylaxis
Grade 3+ hypotension
Grade 3+ AE that does not resolve to grade <2 within 7 days of starting accepted standard of care medical management
Grade 3+ cytokine release syndrome

The following exceptions applied to non-hematologic AEs:
Grade 3 fatigue, nausea, vomiting, or diarrhea that resolves to grade ≤2 with optimal medical management in ≤3 days
Grade 3 fever (as defined by >40° C. for ≤24 hours)
Grade 3 infusion-related reaction that occurs without premedication; subsequent doses should use premedication and if reaction recurs then it will be a DLT
Grade 3 arthralgia or rash that resolves to grade ≤2 within 7 days of starting accepted standard of care medical management (e.g., systemic corticosteroid therapy)
   If a subject had grade 1 or 2 ALT or AST elevation at baseline considered secondhand to liver metastases, a grade 3 elevation must also be ≥3 times baseline and last >7 days.

Serious AEs were defined as any AE that results in any of the following outcomes: Death; Life-threatening AE; Inpatient hospitalization or prolongation of an existing hospitalization; A persistent or significant incapacity or substantial disruption of the ability to conduct normal life functions; or a congenital anomaly/birth defect. Important medical events that may not result in death, be life-threatening, or require hospitalization may be considered serious when, based upon appropriate medical judgment, they may jeopardize the subject and may require medical or surgical intervention to prevent one of the outcomes listed above. Examples of such medical events include allergic bronchospasm requiring intensive treatment in an emergency room or at home, blood dyscrasias or convulsions that do not result in inpatient hospitalization, or the development of drug dependency or drug abuse.

There were no dose-limiting toxicities reported. There was no cumulative toxicity. There were two treatment-related SAEs (1 G3 acute kidney injury and 1 G4 cytokine release syndrome) which resolved with accepted standard of care. Overall, the IL-2 conjugate was considered well-tolerated.

All subjects had at least one treatment-emergent AE (TEAE). TEAEs are detailed in Table 1. No TEAEs were grade 5. Two subjects had a grade 3 event and three subjects had grade 4 events. The grade 3 events included: 1 ALT/AST elevation, 1 neutrophil count decrease, and 1 acute kidney injury. The grade 4 events included: 1 CRS, 1 lymphocyte count increase, and 2 lymphocyte count decreases.

TABLE 1
Treatment Emergent Adverse Events (TEAE) (n=11)

| | IL-2 conjugate | | | |
|---|---|---|---|---|
| | 24 μg/kg | | | |
| | Q3W (N = 11) | | | |
| Primary system organ class | | | | |
| Preferred Term n (%) | All Grades | | Grade ≥3 | |
| Number of Participants with TEAE | 11 | (100) | 8 | (72.7) |
| INFECTIONS AND INFESTATIONS | 1 | (9.1) | 0 | |
| BLOOD AND LYMPHATIC | 2 | (18.2) | 1 | (9.1) |
| SYSTEM DISORDERS | | | | |
| IMMUNE SYSTEM DISORDERS | 2 | (18.2) | 1 | (9.1) |
| METABOLISM AND NUTRITION | 4 | (36.4) | 0 | |
| DISORDERS | | | | |
| PSYCHIATRIC DISORDERS | 1 | (9.1) | 0 | |
| NERVOUS SYSTEM DISORDERS | 3 | (27.3) | 0 | |
| CARDIAC DISORDERS | 2 | (18.2) | 0 | |
| VASCULAR DISORDERS | 3 | (27.3) | 1 | (9.1) |
| RESPIRATORY, THORACIC | 4 | (36.4) | 1 | (9.1) |
| AND MEDIASTINAL | | | | |
| DISORDERS | | | | |
| GASTROINTESTINAL DISORDERS | 4 | (36.4) | 0 | |
| SKIN AND SUBCUTANEOUS | 2 | (18.2) | 0 | |
| TISSUE DISORDERS | | | | |
| MUSCULOSKELETAL AND | 1 | (9.1) | 0 | |
| CONNECTIVE TISSUE | | | | |
| DISORDERS | | | | |
| RENAL AND URINARY DISORDERS | 1 | (9.1) | 1 | (9.1) |
| GENERAL DISORDERS AND | 9 | (81.8) | 1 | (9.1) |
| ADMINISTRATION SITE | | | | |
| CONDITIONS | | | | |
| INVESTIGATIONS | 9 | (81.8) | 6 | (54.5) |
| INJURY, POISONING AND | 0 | | 0 | |
| PROCEDURAL COMPLICATIONS | | | | |

TEAEs mostly consisted of flu-like symptoms, nausea, or vomiting. The TEAEs resolved with accepted standard of care. Treatment-related AEs were transient. AEs of fever, hypotension, and hypoxia did not correlate with IL-5/IL-6 cytokine elevation. One subject presented with IL-6 elevation at 24 hours to 1,000 μg/mL (post tocilizumab treatment), which declined to below 100 μg/mL by 72 hours. There was no notable impact to vital signs, no QTc prolongation, or other cardiac toxicity.

Accordingly, the IL-2 conjugate demonstrated encouraging PD data and was generally well-tolerated. It was determined that the in vivo half-life of the IL-2 conjugate was about 10 hours. Overall, the results are considered to support non-alpha preferential activity of the IL-2 conjugate, with a tolerable safety profile, encouraging PD and preliminary evidence of activity in patients with immune-sensitive tumors.

Cohorts Using 8 μg/kg and 16 μg/kg Doses [Q3W]

The IL-2 conjugate was administered via IV infusion at a dose of 8 μg/kg or 16 μg/kg for 30 minutes every 3 weeks [Q3W]. Effects on the same biomarkers described above for the studies using a 24 μg/kg dose were analyzed as surrogate predictors of safety and/or efficacy. Subjects in these studies met the same criteria as the subjects receiving the 24 μg/kg dose.

Cohort 1: 8 μg/kg [Q3W] dosing.

Cohort 1 (individuals having malignant solid tumors) received the IL-2 conjugate at an 8 μg/kg dose Q3W for five dose cycles.

Four individuals that had initial disease stabilization (at the 6-week assessment; one patient had ~12% tumor regression) were treated with the IL-2 conjugate. These four subjects had post-dose CD8+ Ki67 expression levels that exceeded 60 percent (65%-80%).

Biomarkers were determined for 4 individuals in Cohort 1 as follows. The peripheral expansion of CD8+ T effector cells averaged 1.53-fold above baseline; one subject was 2.1-fold above baseline. All four subjects had post-dose NK Cell Ki67 expression levels of nearly 100 percent. All four subjects had post-dose peripheral expansion of NK cells that averaged 3.9-fold above baseline at day 3; one subject was 5.0-fold above baseline at day 3. There were no changes in the PK parameters from cycle 1 to cycle 2. There were no anti-drug antibodies detected in the first three subjects; these were measured out to cycle 5 for two subjects and out to cycle 4 for one subject.

Serum IFNγ, IL-6, and IL-5 levels were measured at 1, 2, and 3 days post-dosing during cycles 1 and 2. Means and ranges are shown in Table 2. The top values of the range were observed 1 day post-dosing for all subjects.

TABLE 2
Safety/Toxicity Biomarkers - Cytokine
Summary Cohort 1: Q3W, 8 μg/kg

| | IFNγ pg/mL | IL-6 pg/mL | IL-5 pg/mL |
|---|---|---|---|
| | Mean (range) | Mean (range) | Mean (range) |
| Subject | BLQ <3.5 | BLQ <1.3 | BLQ <8.8 |
| 2001-0001 | 29.6 (BLQ-66.9) | 2.6 (BLQ-6.5) | BLQ |
| | | | (All points BLQ) |
| 2001-0002 | 19.9 (3.7-90.4) | 3.1 (1.33-11.6) | BLQ |
| | | | (All points BLQ) |
| 2001-0003 | 21.3 (5.6-53.5) | 3.3 (BLQ-5.96) | BLQ |
| | | | (All points BLQ) |
| 2001-0004 | 38.1 (BLQ-60.8) | 4 (BLQ-6.5) | BLQ |
| | | | (All points BLQ) |

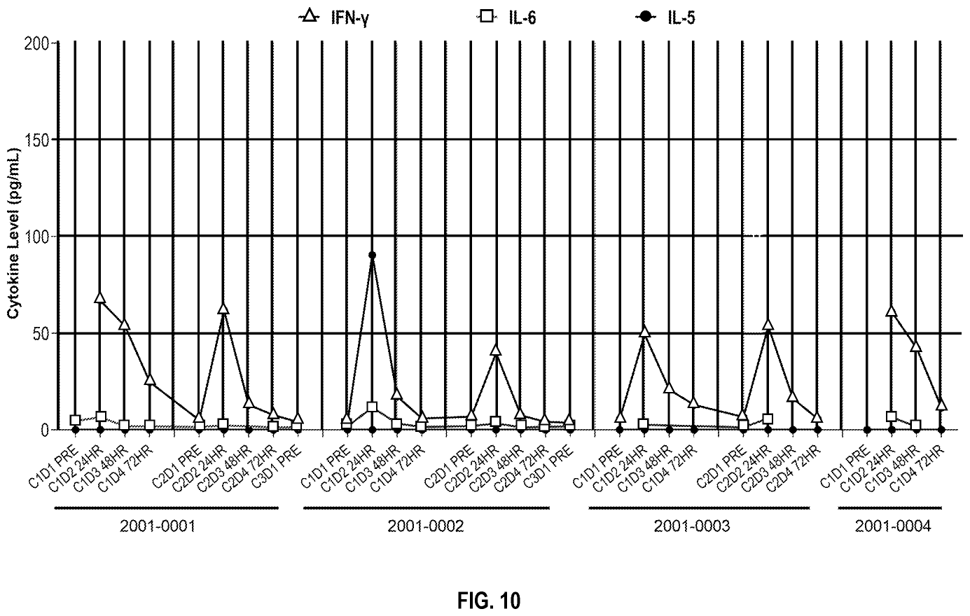

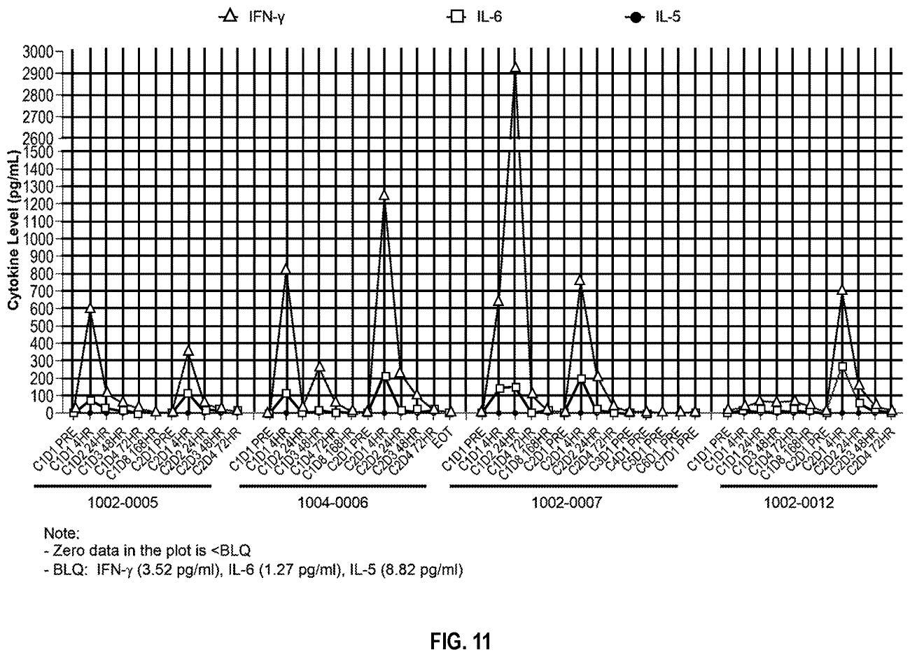

Measured cytokine levels are shown graphically in FIG. 10.

It was reported in Teachey et al., Cancer Discov. 2016; 6 (6); 664-79, that in acute lymphoblastic leukemia patients treated with CAR-T cells, severe cytokine release syndrome (CRS level 4 or 5) was associated with higher values of each of the three cytokines measured in the present study than nonsevere cytokine release syndrome (CRS level 0-3). Data from Teachey et al. for IFNγ, IL-6, and IL-5, expressed as median (range), are reproduced in Table 3.

TABLE 3
IFNγ, IL-6, and IL-5 levels reported
as associated with CRS levels 0-3 and 4-5.

| CRS | IFNγ | | IL-6 | | IL-5 | |
|---|---|---|---|---|---|---|
| level | pg/mL | | pg/mL | | pg/mL | |
| 0-3 | 34.1 | (2.14-8,233) | 122 | (0.40-20,892) | 4.25 | (0.39-264) |
| 4-5 | 3,119 | (160-15,482) | 8,309 | (580-102,476) | 15.3 | (1.71-333) |

Accordingly, the results in Table 2 and FIG. 10 are consistent with absence of severe CRS.

The IL-2 conjugate demonstrated encouraging PD data and was generally well-tolerated. Overall, the results are considered to support non-alpha preferential activity of the IL-2 conjugate, with a tolerable safety profile, encouraging PD and preliminary evidence of activity in patients with immune-sensitive tumors.

Cohort 2: 16 μg/kg [Q3W] dosing.

This example reports results for up to 6 individuals having malignant solid tumors who received the IL-2 conjugate at a 16 μg/kg dose Q3W for at least 2 cycles. After the first dose, one subject had a post dose peripheral expansion of CD8+ T effector cells of 4.1-fold; the average across the three patients was 2.2-fold expansion. All three subjects had post-dose peripheral expansion of NK cells that exceeded 4-fold above baseline at day 3; one subject was 11.4-fold above baseline and the average was 7.2-fold.

Serum IFNγ, IL-6, and IL-5 levels were measured at 1, 2, and 3 days post-dosing during Cycles 1 and 2. Means and ranges are shown in Table 4. The top values of the range were observed 1 day post-dosing for the indicated 3 subjects.

TABLE 4
Safety/Toxicity Biomarkers - Cytokine
Summary Cohort 2: Q3W, 16 μg/kg

| | IFNγ pg/mL | | IL-6 pg/mL | | IL-5 pg/mL |
|---|---|---|---|---|---|
| | Mean (range) | | Mean (range) | | Mean (range) |
| Subject | BLQ <3.5 | | BLQ <1.3 | | BLQ <8.8 |
| 1002-0005 | 91 | (BLQ-349) | 29.2 | (3-114) | BLQ (All |
| | | | | | points BLQ) |
| 1004-0006 | 196.8 | (BLQ-817) | 21 | (1.5-112) | BLQ (All |
| | | | | | points BLQ) |
| 1002-0007 | 190.1 | (5.4-641) | 62.5 | (BLQ-153) | BLQ (All |
| | | | | | points BLQ) |

Measured cytokine levels for 4 subjects are shown graphically in FIG. 11. These results are also consistent with absence of severe CRS.

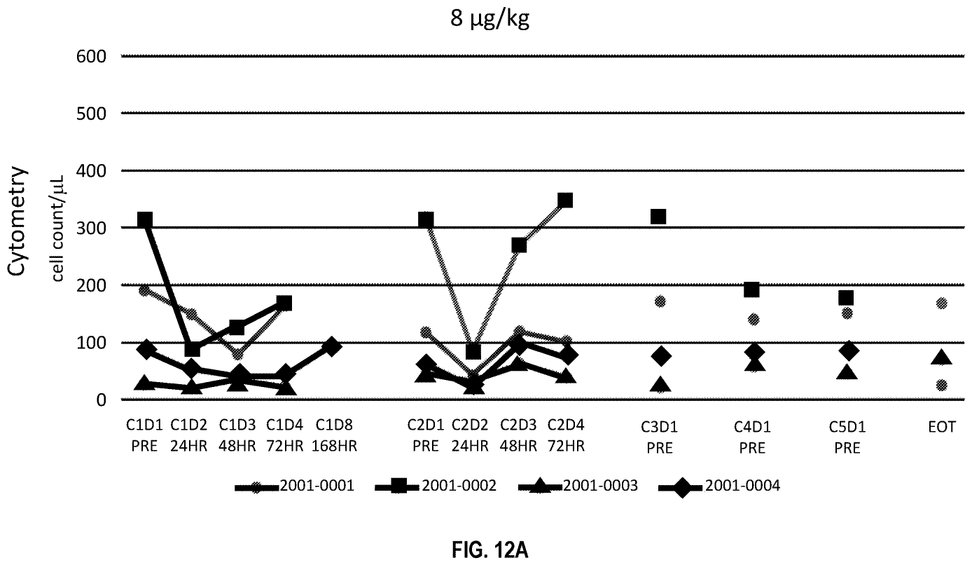

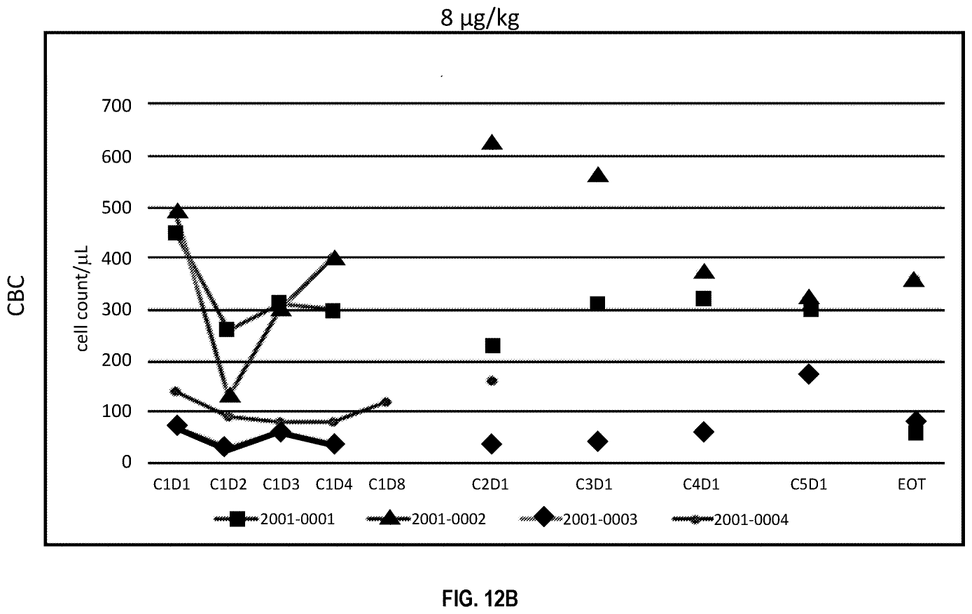

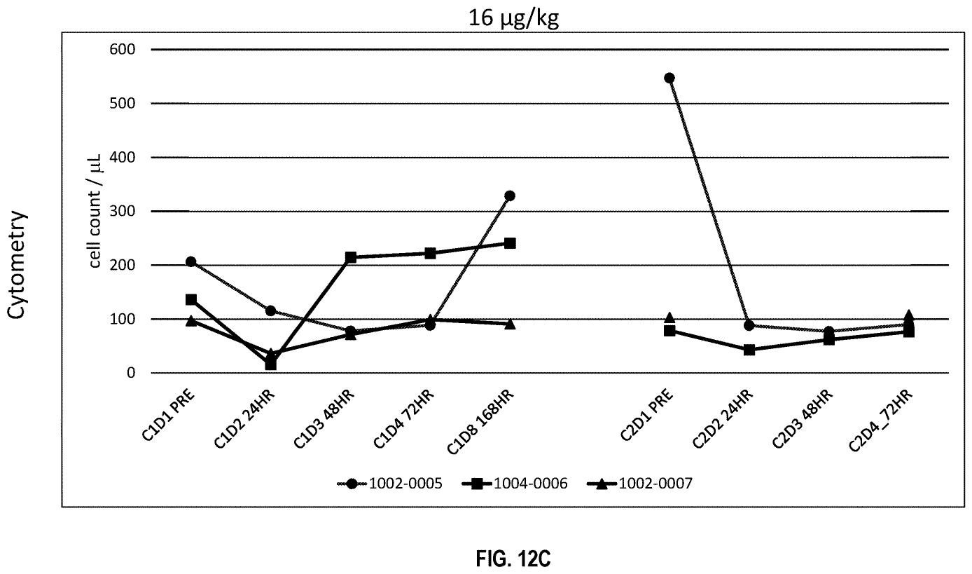

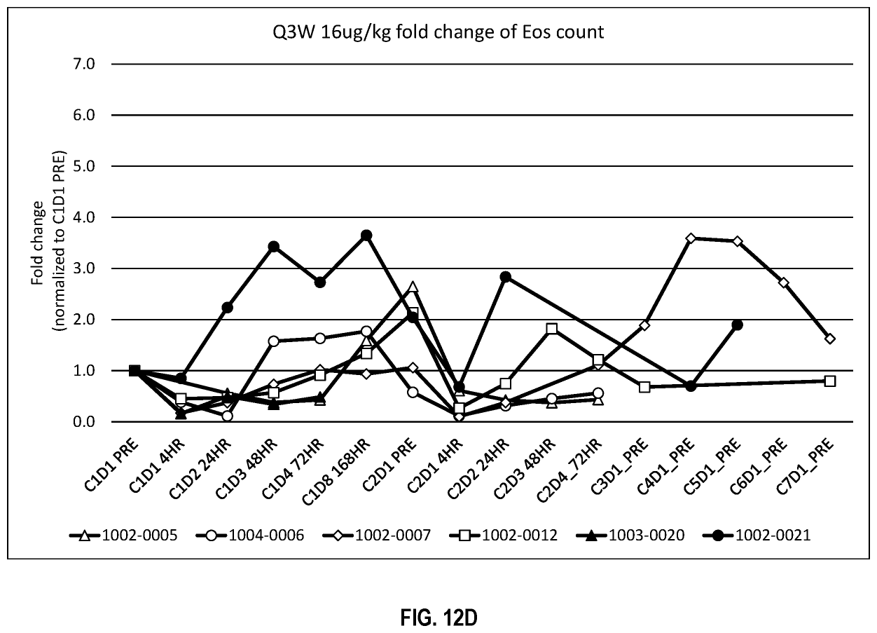

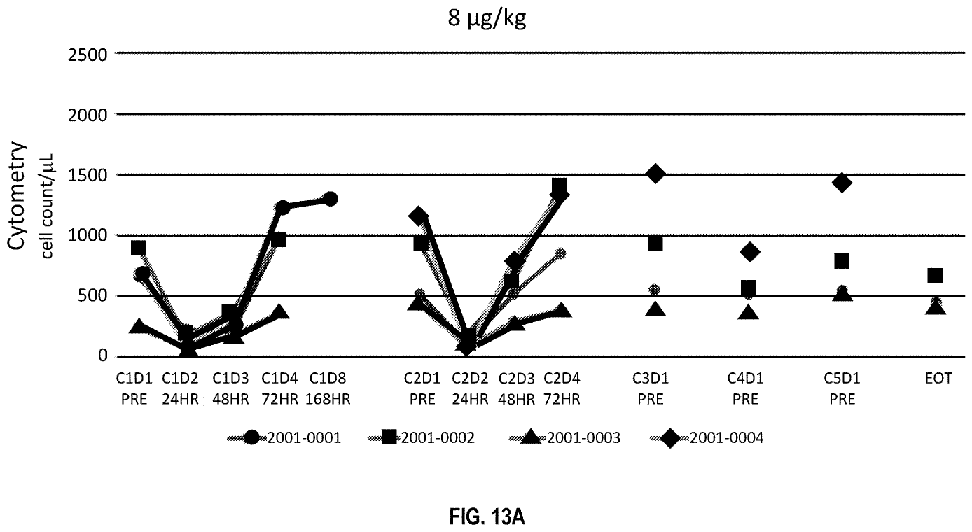

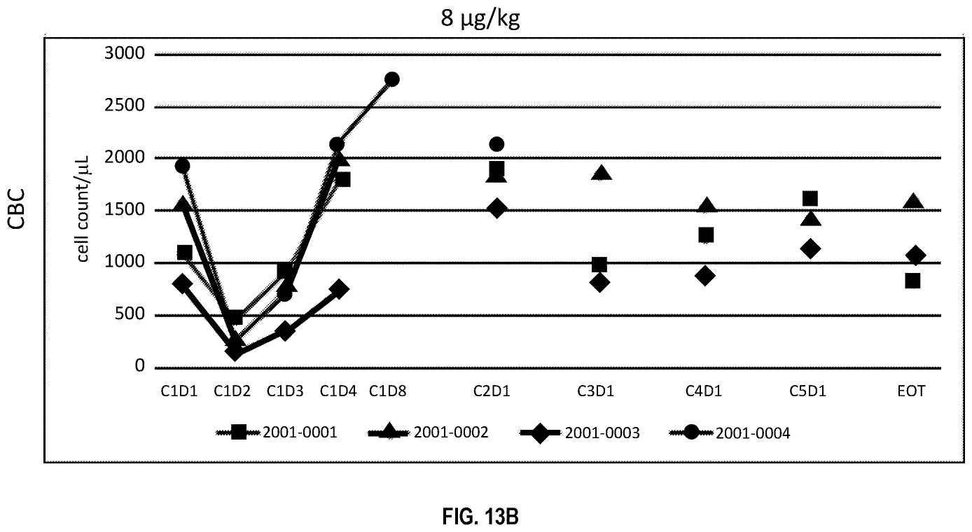

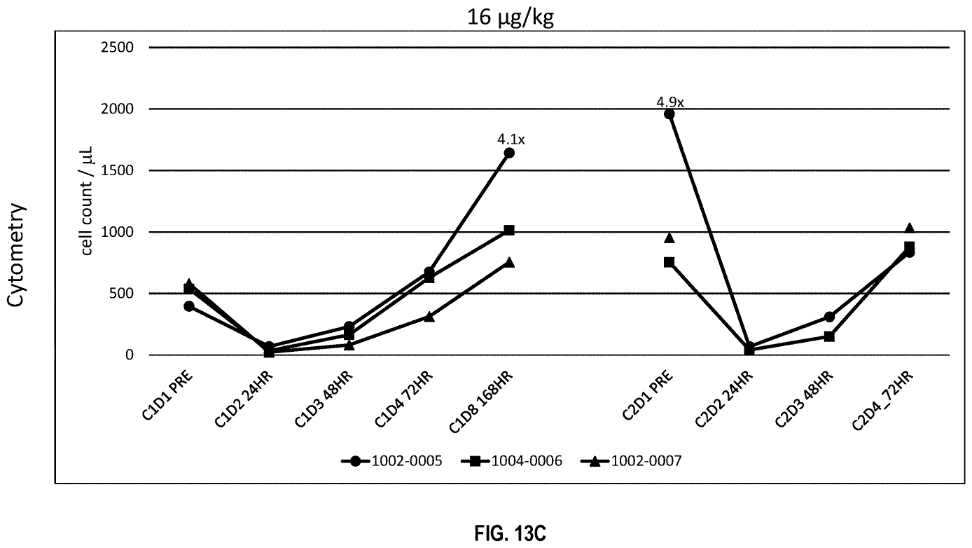

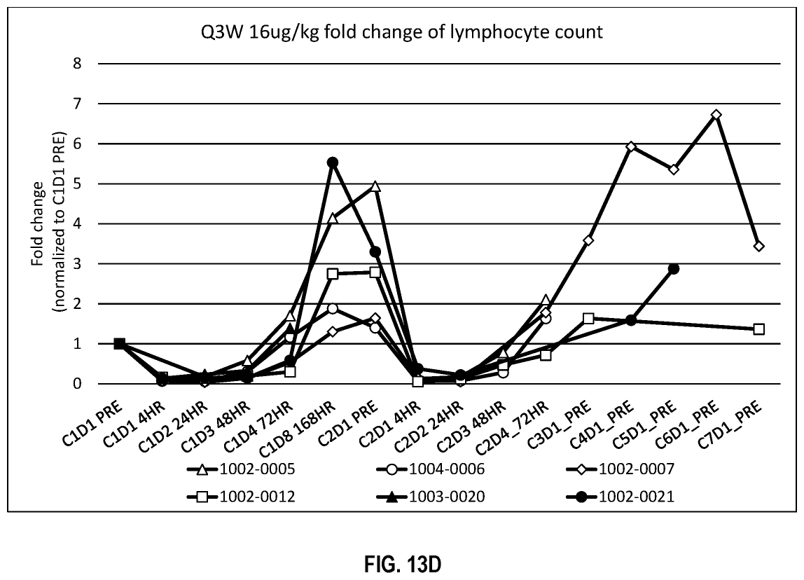

Eosinophil counts were measured by FACS and CBC for cohorts 1-2 (FIGS. 12A-12D). The measured values were consistently below the range of 2328-15958 eosinophils/μL in patients with IL-2 induced eosinophilia as reported in Pisani et al., Blood 1991 Sep. 15; 78 (6): 1538-44. Peripheral lymphocyte count was also measured for Cohorts 1 and 2 (FIGS. 13A-13D).

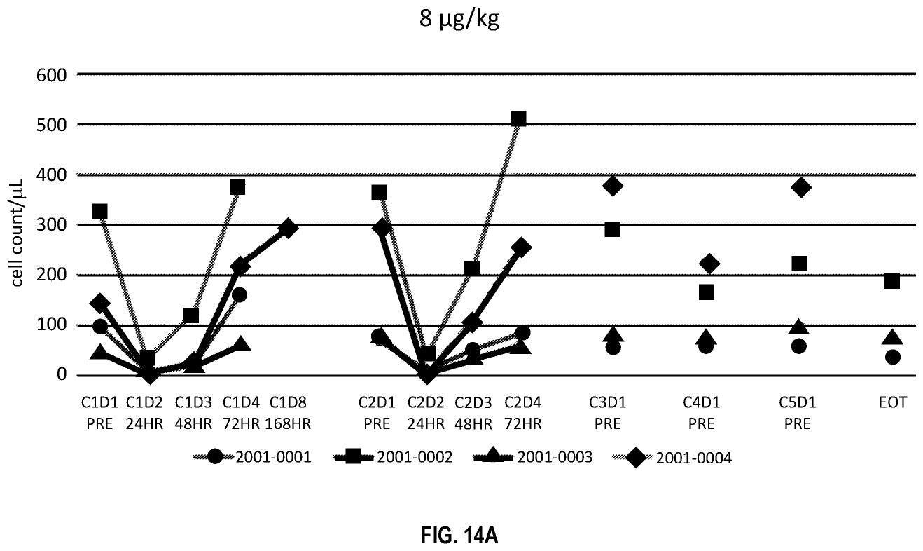

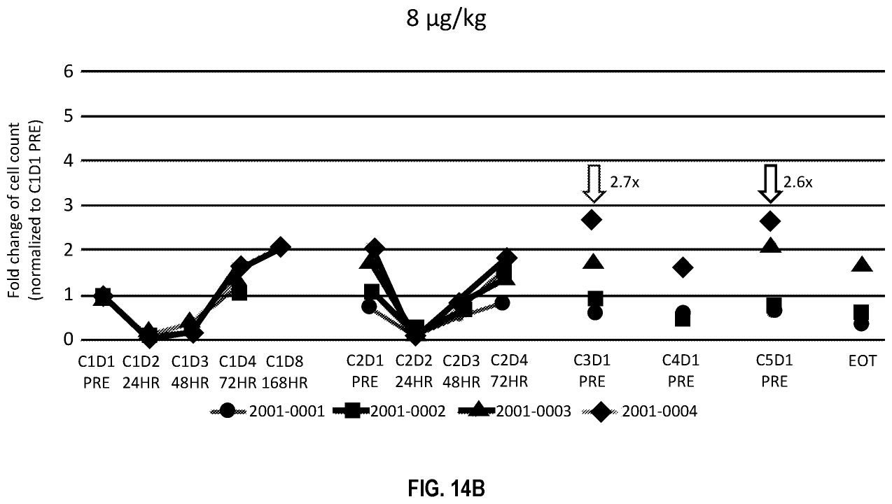

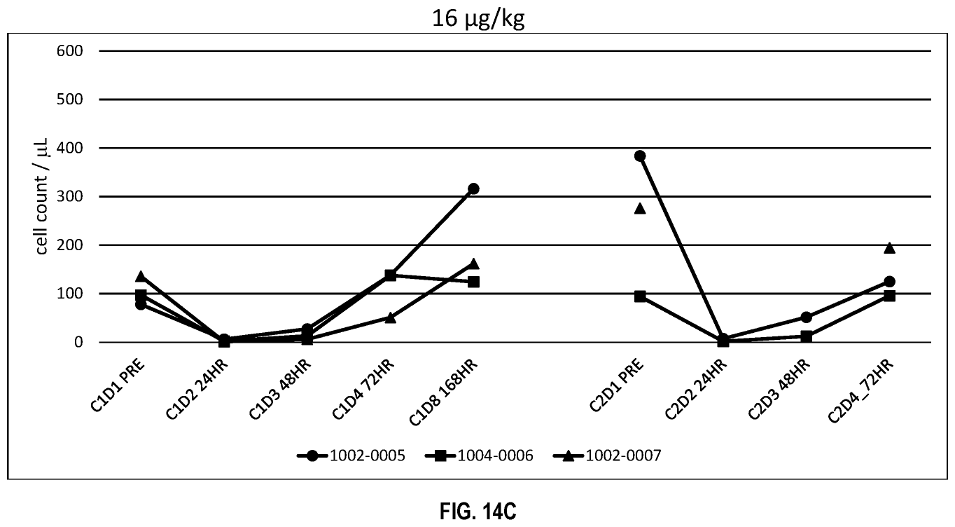

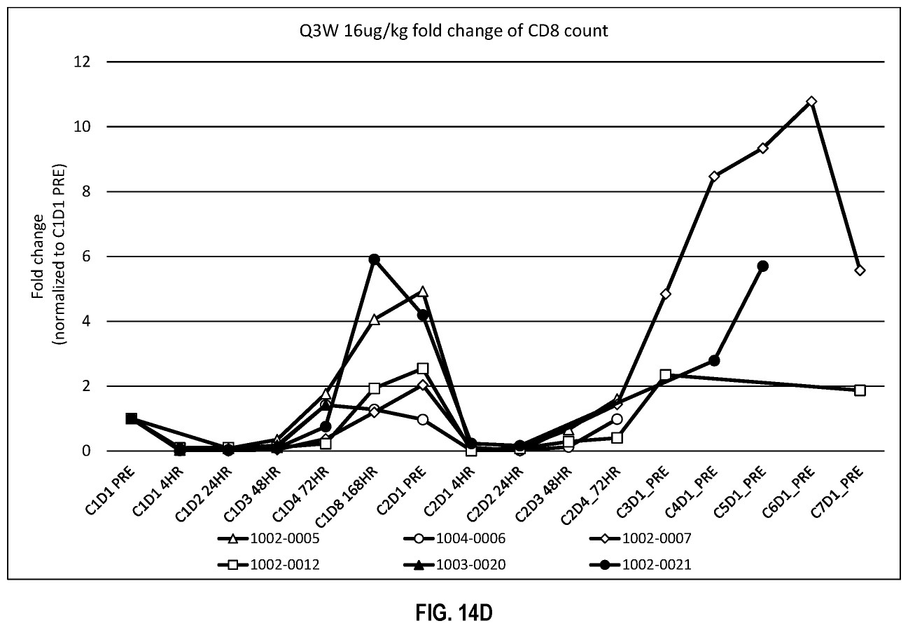

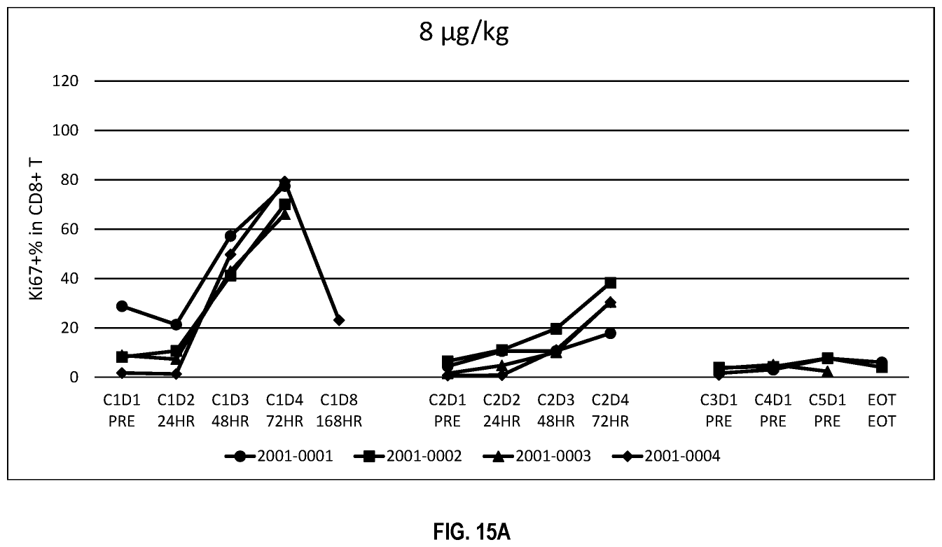

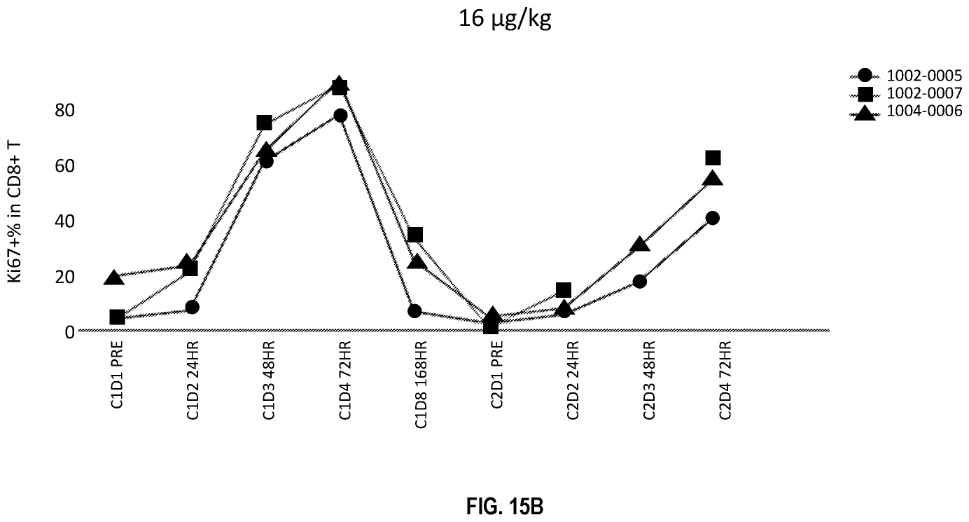

Efficacy biomarkers for Cohorts 1 and 2. Peripheral CD8+ T_{eff} Counts were measured for Cohorts 1 and 2 (FIGS. 14A-14D). Prolonged CD8+ expansion over baseline (e.g., greater than or equal to 2-fold change) was observed at 3 weeks after the previous dose in some subjects. The percentage of CD8+ T_{eff} cells expressing Ki67 was also measured for Cohorts 1 and 2 (FIGS. 15A-15B).

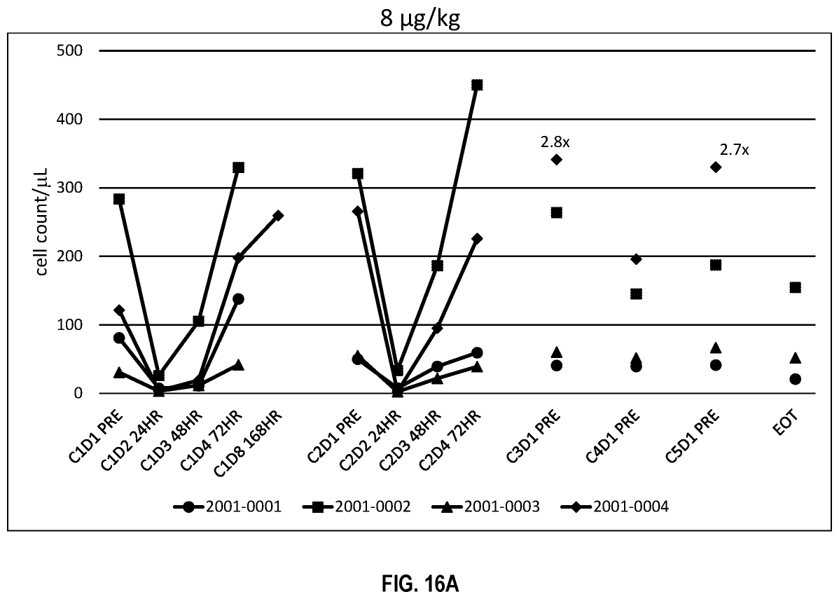

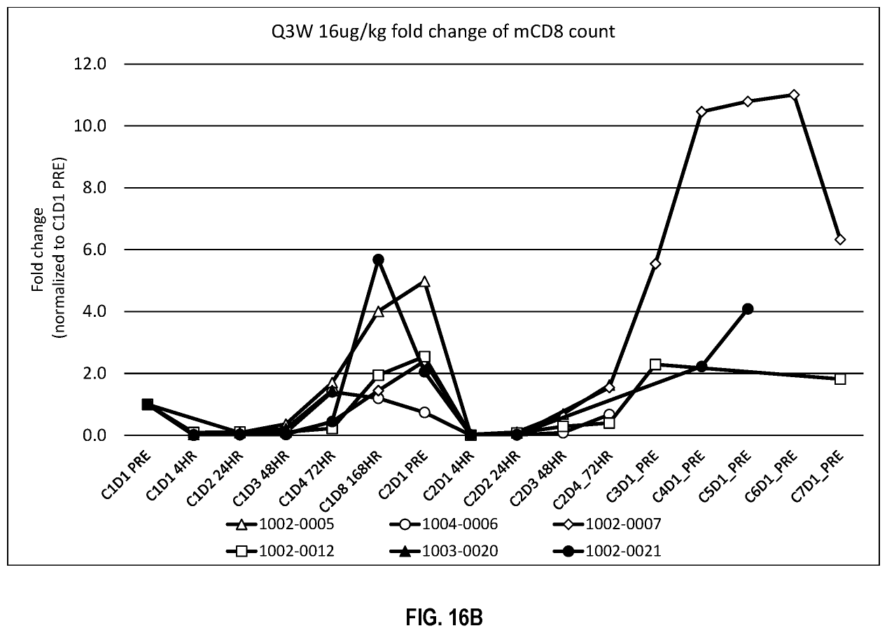

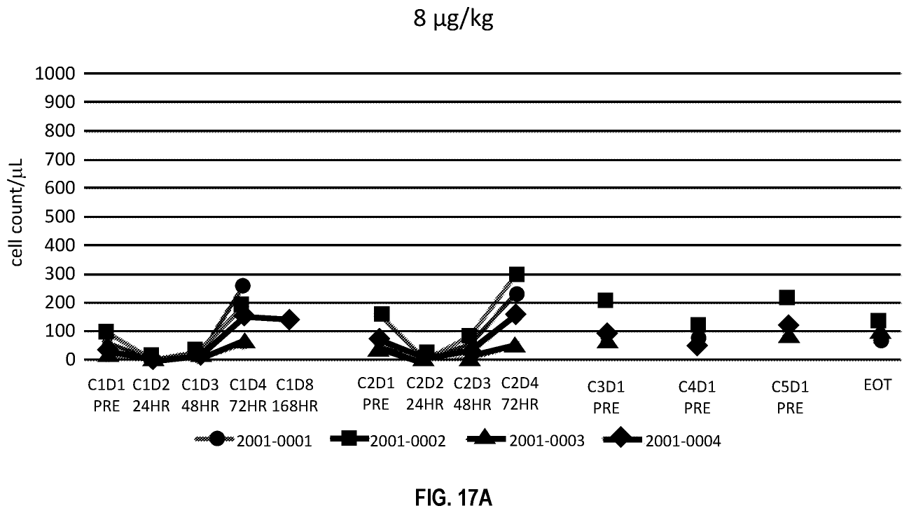

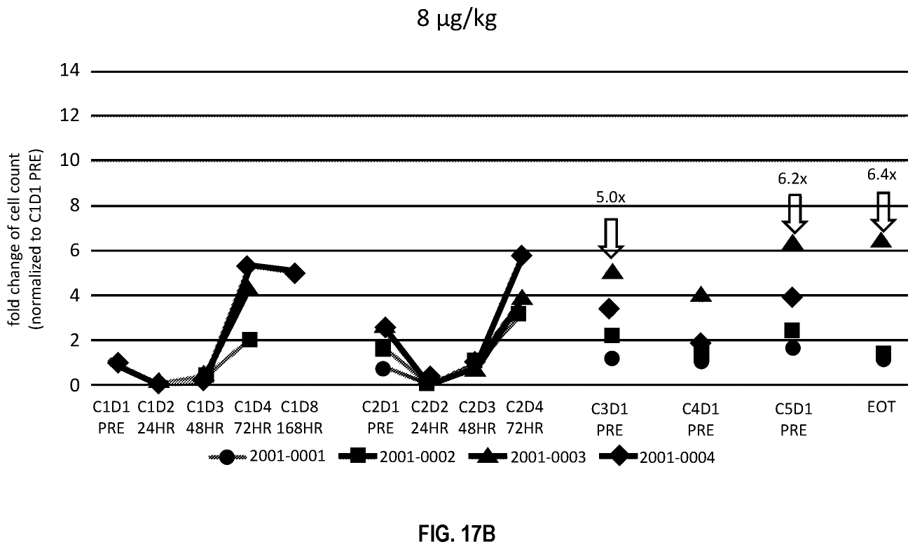

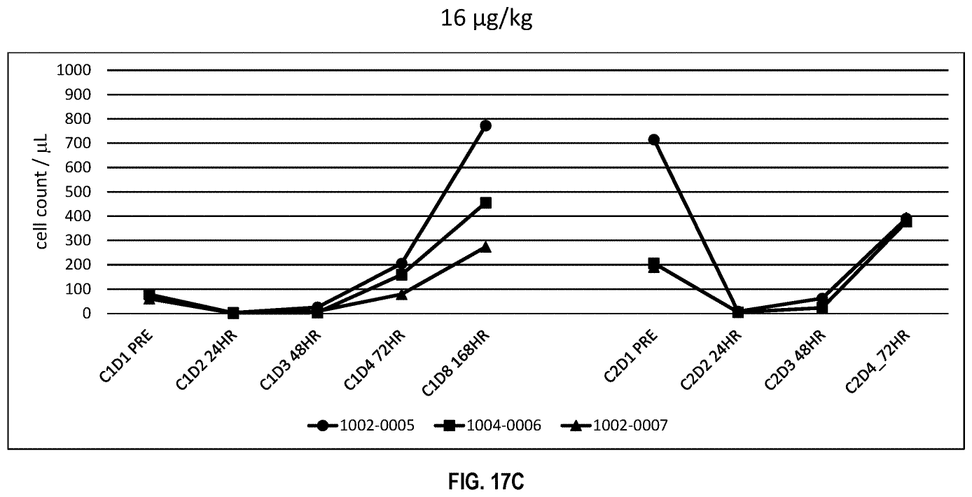

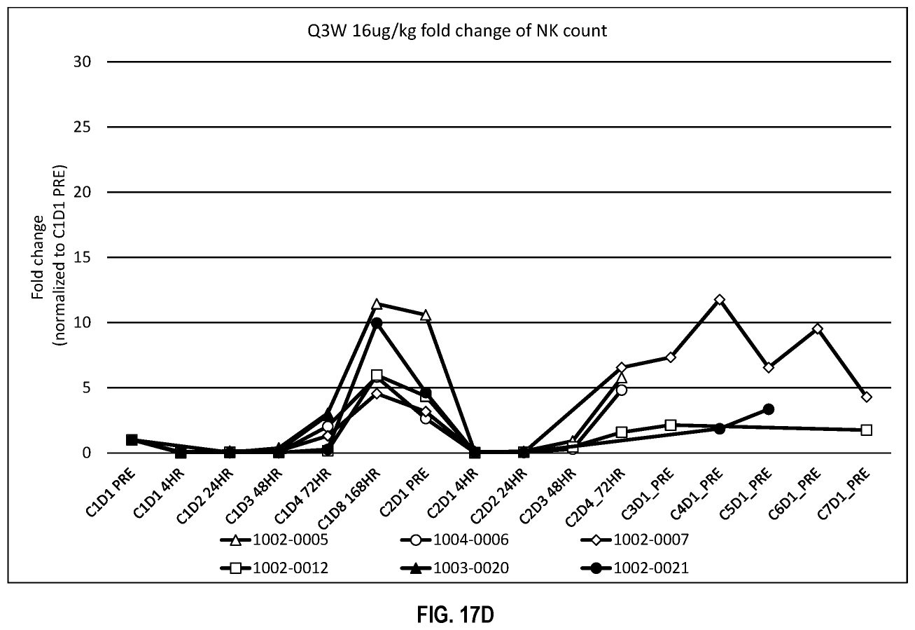

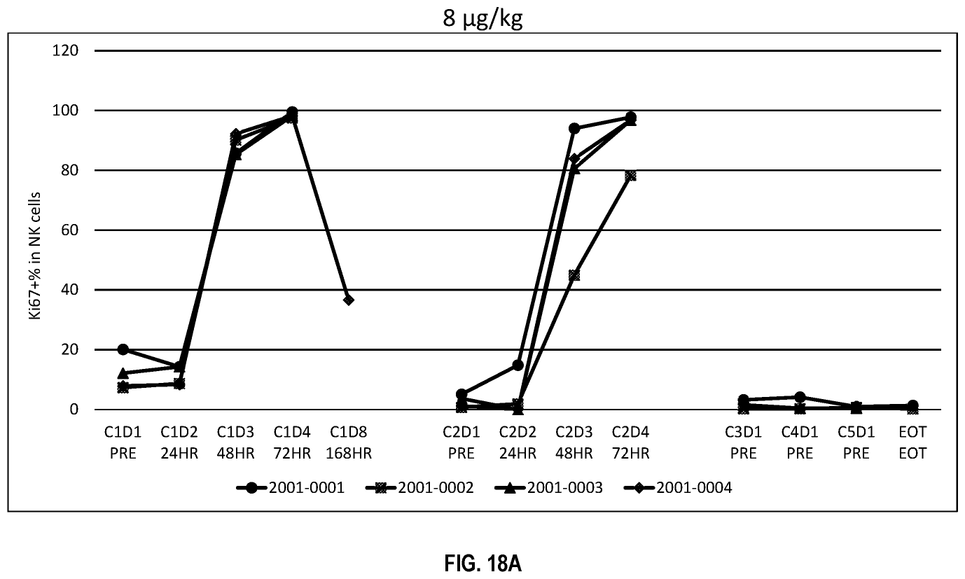

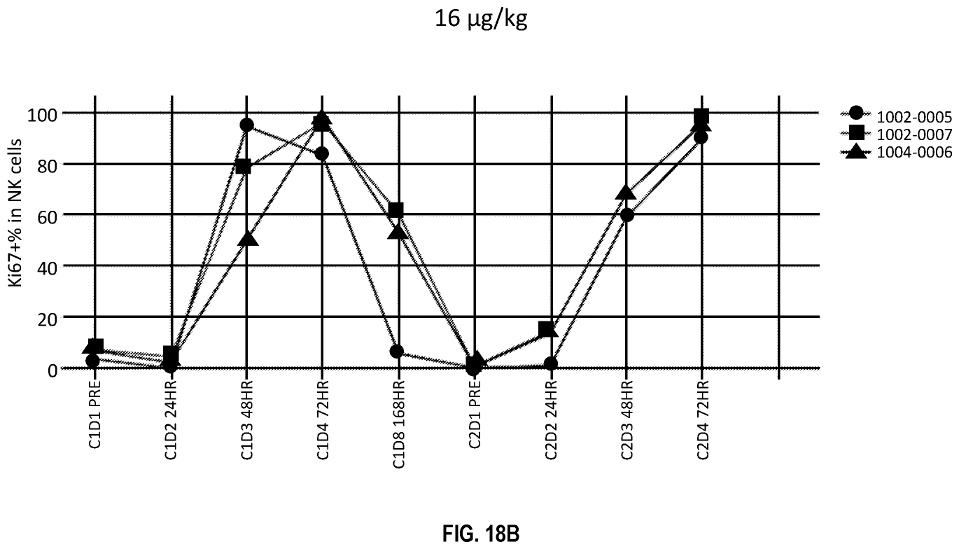

Peripheral memory CD8+ counts are shown in FIGS. 16A-16B. Peripheral NK cell counts are shown in FIGS. 17A-17D. Prolonged NK cell expansion over baseline (e.g., greater than or equal to 5-fold change) was observed at 3 weeks after the previous dose in some subjects. The percentage of NK cells expressing Ki67 was also measured for Cohorts 1 and 2 (FIGS. 18A-18B).

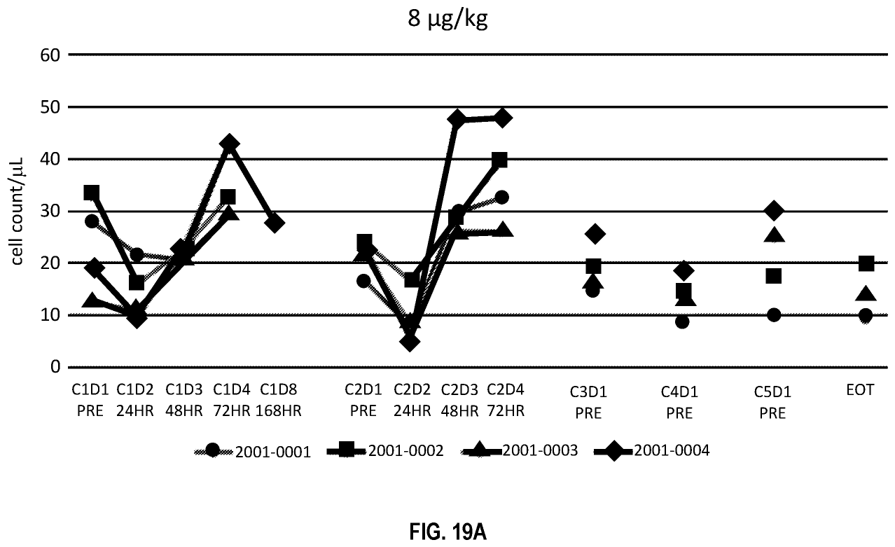

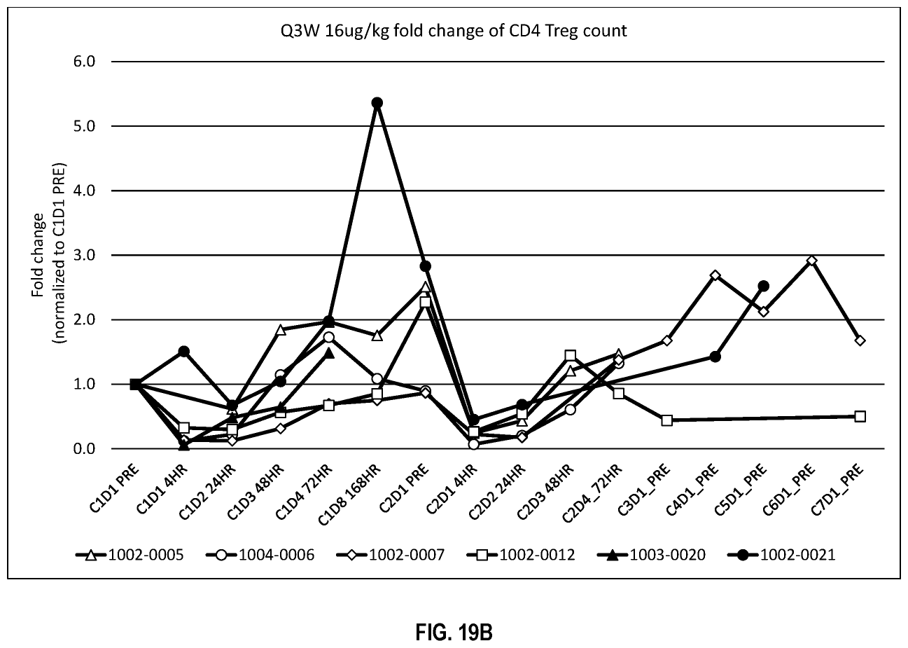

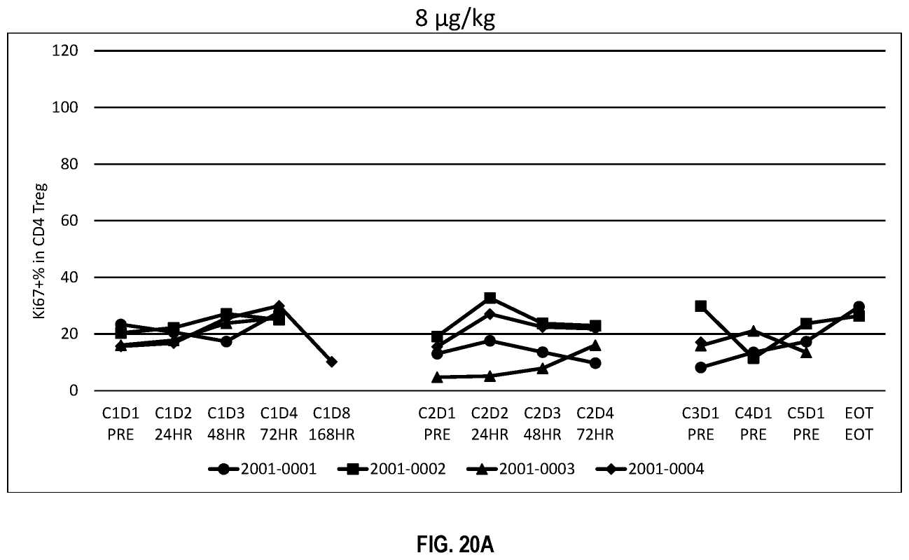

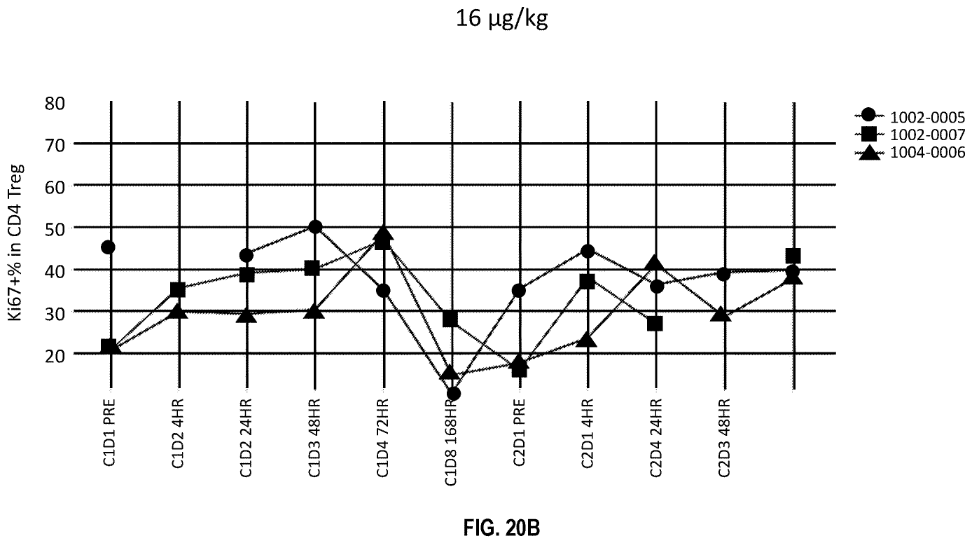

Peripheral CD4+ T_{reg} counts for Cohorts 1 and 2 are shown in FIGS. 19A-19B. The percentage of CD4+ T_{reg} cells expressing Ki67 was also measured for Cohorts 1 and 2 (FIGS. 20A-B).

Summary of Results; Discussion. The subjects discussed above receiving the 8 μg/kg dose had post-dose CD8+ Ki67 expression levels exceeding 60% (65%-80%), with peripheral expansion of CD8+ T effector (Teff) cells averaging 1.53-fold above baseline. All 4 subjects also had post-dose NK cell Ki67 expression levels of nearly 100%, with peripheral expansion of NK cells averaging 3.9-fold above baseline at day 3. Of the 3 subjects discussed above who received 16 μg/kg doses, 1 had a post-dose peripheral expansion of CD8+ Teff cells 4.1-fold above baseline at day 7; the average expansion across the 3 subjects was 2.2-fold. There was no observation of anti-drug antibodies (IL-2 or PEG), and no meaningful elevations in IL-5 and IL-6 levels. Also, the PK data does not indicate a decrease in AUC from cycle 1 to cycle 2 (data not shown).

There were no dose-limiting toxicities reported at either dose and there were no treatment-related adverse events (TRAE) leading to discontinuation or treatment-related serious AEs reported.

TEAEs for 10 subjects receiving Q3W 8 or 16 μg/kg doses are detailed in Table 5. No TEAEs were Grade 5. Two subjects had a Grade 4 event (one AST elevation and one lymphocyte count decrease).

TABLE 5
Treatment Emergent Adverse Events (TEAE)

| | IL-2 conjugate | | | | IL-2 conjugate | | | |
|---|---|---|---|---|---|---|---|---|
| | 8 μg/kg | | | | 16 μg/kg | | | |
| | Q3W (N = 4) | | | | Q3W (N = 6) | | | |
| Primary system organ class | | | | | | | | |
| Preferred Term n (%) | All Grades | | Grade ≥3 | | All Grades | | Grade ≥3 | |
| Number of Participants with | 4 | (100) | 1 | (25.0) | 6 | (100) | 2 | (33.3) |
| TEAE | | | | | | | | |
| INFECTIONS AND | 0 | | 0 | | 0 | | 0 | |
| INFESTATIONS | | | | | | | | |
| BLOOD AND LYMPHATIC | 1 | (25.0) | 0 | | 2 | (33.3) | 0 | |
| SYSTEM DISORDERS | | | | | | | | |
| IMMUNE SYSTEM | 0 | | 0 | | 0 | | 0 | |
| DISORDERS | | | | | | | | |
| METABOLISM AND | 0 | | 0 | | 1 | (16.7) | 0 | |
| NUTRITION DISORDERS | | | | | | | | |
| PSYCHIATRIC DISORDERS | 0 | | 0 | | 0 | | 0 | |
| NERVOUS SYSTEM | 0 | | 0 | | 2 | (33.3) | 0 | |
| DISORDERS | | | | | | | | |
| CARDIAC DISORDERS | 1 | (25.0) | 0 | | 2 | (33.3) | 0 | |
| VASCULAR DISORDERS | 1 | (25.0) | 0 | | 1 | (16.7) | 0 | |
| RESPIRATORY, THORACIC | 2 | (50.0) | 0 | | 1 | (16.7) | 0 | |
| AND MEDIASTINAL | | | | | | | | |
| DISORDERS | | | | | | | | |
| GASTROINTESTINAL | 4 | (100) | 0 | | 4 | (66.7) | 0 | |
| DISORDERS | | | | | | | | |
| SKIN AND SUBCUTANEOUS | 4 | (100) | 1 | (25.0) | 2 | (33.3) | 0 | |
| TISSUE DISORDERS | | | | | | | | |
| MUSCULOSKELETAL AND | 1 | (25.0) | 0 | | 0 | | 0 | |
| CONNECTIVE TISSUE | | | | | | | | |
| DISORDERS | | | | | | | | |
| RENAL AND URINARY | 0 | | 0 | | 0 | | 0 | |
| DISORDERS | | | | | | | | |
| GENERAL DISORDERS AND | 4 | (100) | 0 | | 5 | (83.3) | 0 | |
| ADMINISTRATION SITE | | | | | | | | |
| CONDITIONS | | | | | | | | |
| INVESTIGATIONS | 0 | | 0 | | 5 | (83.3) | 2 | (33.3) |
| INJURY, POISONING AND | 0 | | 0 | | 0 | | 0 | |
| PROCEDURAL | | | | | | | | |
| COMPLICATIONS | | | | | | | | |

TEAEs mostly consisted of flu-like symptoms, nausea, or vomiting. The TEAEs resolved with accepted standard of care. Treatment-related AEs were transient. AEs of fever, hypotension, and hypoxia did not correlate with IL-5/IL-6 cytokine elevation. There was no notable impact to vital signs, no QTc prolongation, or other cardiac toxicity. Accordingly, the IL-2 conjugate demonstrated encouraging PD data and was generally well-tolerated. It was determined that the in vivo half-life of the IL-2 conjugate was about 10 hours. Overall, the results are considered to support non-alpha preferential activity of the IL-2 conjugate, with a tolerable safety profile, encouraging PD and preliminary evidence of activity in patients with immune-sensitive tumors.

Selected individual results. One subject having prostate adenocarcinoma received 10 cycles of Q3W 16 μg/kg doses and showed stable disease (24% decrease after two cycles). This subject came off treatment after the 10th cycle due to rising PSA.

One subject having non-small cell lung cancer received at least 6 cycles of Q3W 16 μg/kg doses and showed stable disease (17.9% decrease after 5 cycles).

Anti-drug Antibodies (ADAs). Samples from treated subjects were assayed after each dose cycle for anti-drug antibodies (ADAs). Anti-polyethylene glycol autoantibodies were detected by direct immunoassays (detection limit: 36 ng/mL). A bridging MesoScale Discovery ELISA was performed with a labeled form of the IL-2 conjugate, having a detection limit of 4.66 ng/mL. Additionally, a cell-based assay for neutralizing antibodies against the IL-2 conjugate was performed using the CTLL-2 cell line, with STAT5 phosphorylation as the readout (detection limit: 6.3 μg/mL).

Samples were collected and analyzed after each dose cycle from two subjects who received 5 dose cycles and one subject who received 4 dose cycles. An assay-specific cut point was determined during assay qualification as a signal to negative ratio of 1.09 or higher for the IL-2 conjugate ADA assay and 2.08 for the PEG ADA assay. Samples that gave positive or inconclusive results in the IL-2 conjugate assay were subjected to confirmatory testing in which samples and controls were assayed in the presence and absence of confirmatory buffer (10 μg/mL IL-2 conjugate in blocking solution). Samples that gave positive or inconclusive results in the PEG assay were subjected to confirmatory testing in which samples and controls were assayed in the presence and absence of confirmatory buffer (10 μg/mL IL-2 conjugate in 6% horse serum). Samples will be considered “confirmed” if their absorbance signal is inhibited by equal to or greater than an assay-specific cut point determined during assay qualification (14.5% for the IL-2 conjugate or 42.4% for PEG) in the detection step. No confirmed ADA against the IL-2 conjugate or PEG were detected (data not shown).

### Example 3. Clinical Study of Biomarker Effects Following IL-2 Conjugate Administration (32 μg/kg [Q3W])

An extension of the study above regarding the 24 μg/kg dose was performed to characterize immunological effects of in vivo administration of an IL-2 conjugate administered via IV infusion at a dose of 32 μg/kg for 30 minutes every 3 weeks [Q3W]. Effects on the same biomarkers described in Example 2 were analyzed as surrogate predictors of safety and/or efficacy.

Subjects in this second cohort met the same criteria as the subjects in Example 2. Tumor types included cervical, colorectal, pancreatic, and sarcoma.

Q3W dosing. Six individuals (5 [83.3%] male, 4 [66.7%] Caucasian) having advanced or metastatic solid tumors received the IL-2 conjugate at a 32 μg/kg dose Q3W (1 dose per cycle). Here and throughout the examples, drug mass per kg subject (e.g., 32 μg/kg) refers to IL-2 mass exclusive of PEG and linker mass.

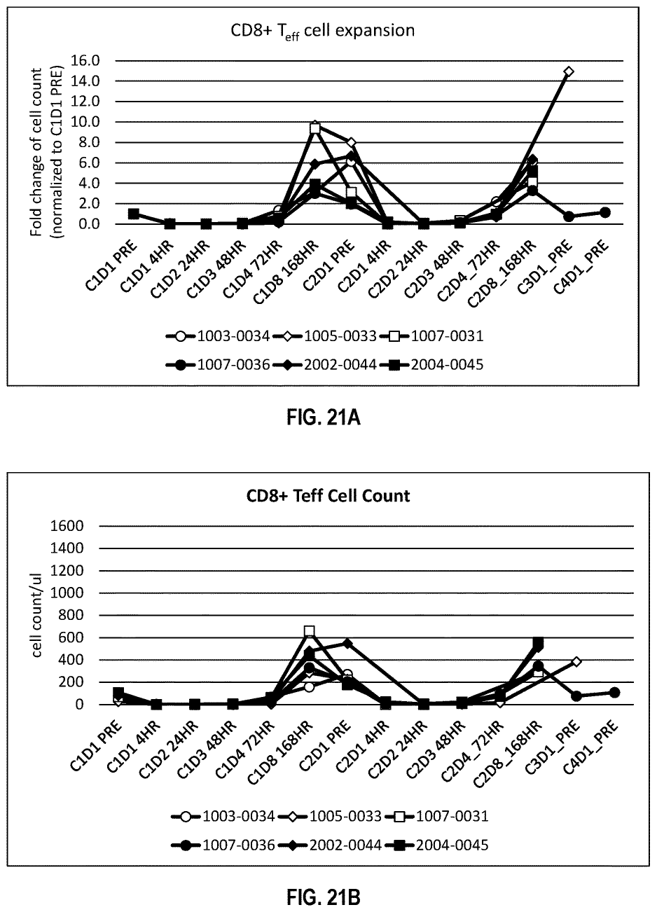

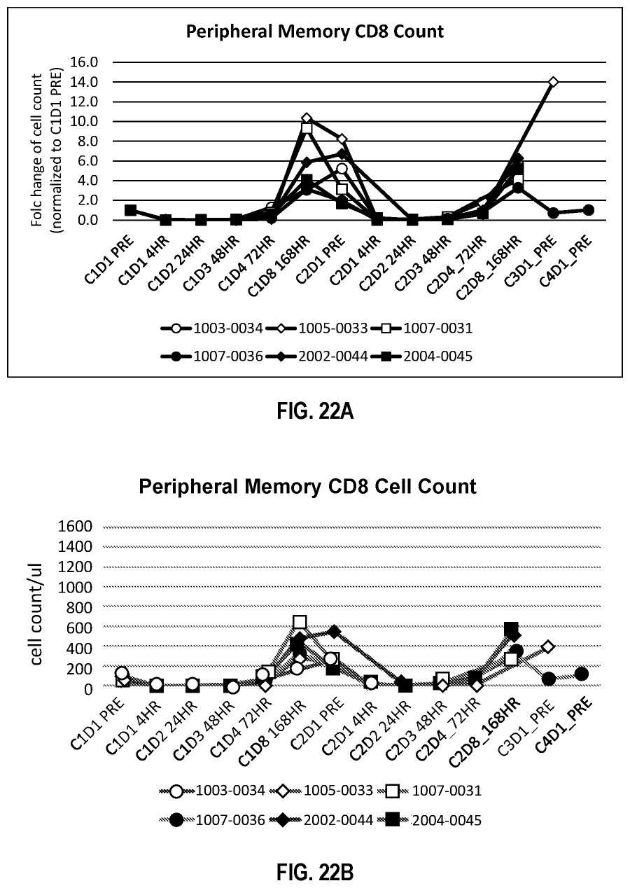

Efficacy biomarkers. Peripheral CD8+ T_{eff} cell counts were measured (FIGS. 21A-21B). Prolonged CD8+ expansion over baseline (e.g., greater than or equal to 4-fold change) was observed at 3 weeks in some subjects. Peripheral CD8+ memory cells counts are shown in FIGS. 22A-22B.

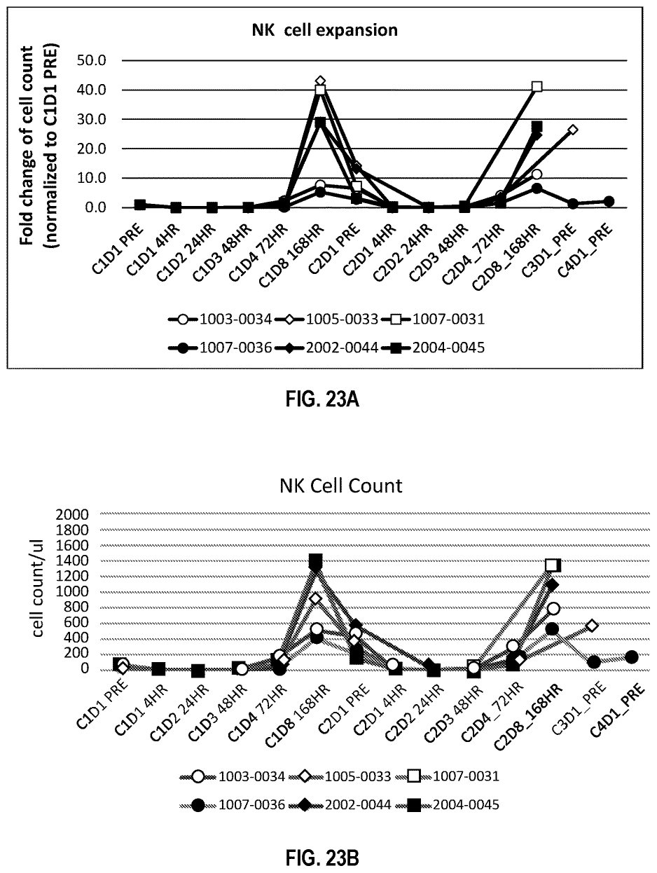

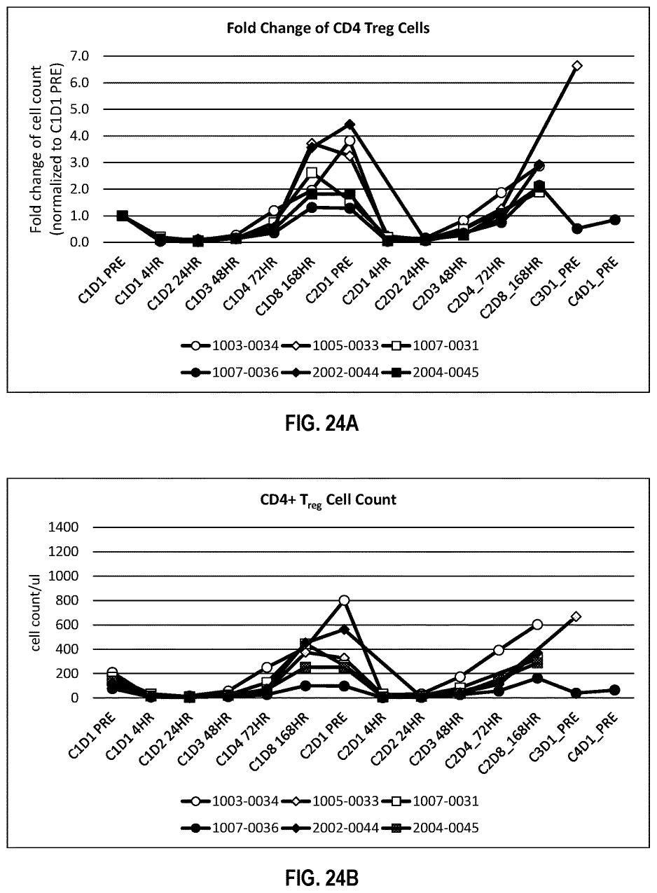

Peripheral NK cell counts are shown in FIGS. 23A-23B. An increase in NK cell count was observed in each subject.

Peripheral CD4+ T_{reg} counts are shown in FIGS. 24A-24B.

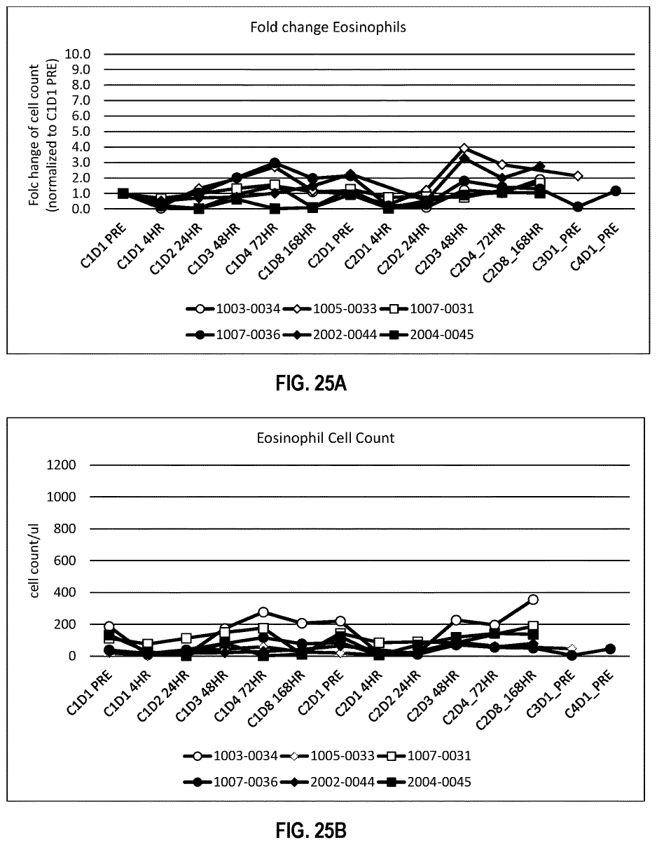

Eosinophil counts were measured (FIGS. 25A-25B). The measured values did not exceed a four-fold increase and were consistently below the range of 2328-15958 eosinophils/μL in patients with IL-2 induced eosinophilia as reported in Pisani et al., Blood 1991 Sep. 15; 78 (6): 1538-44.

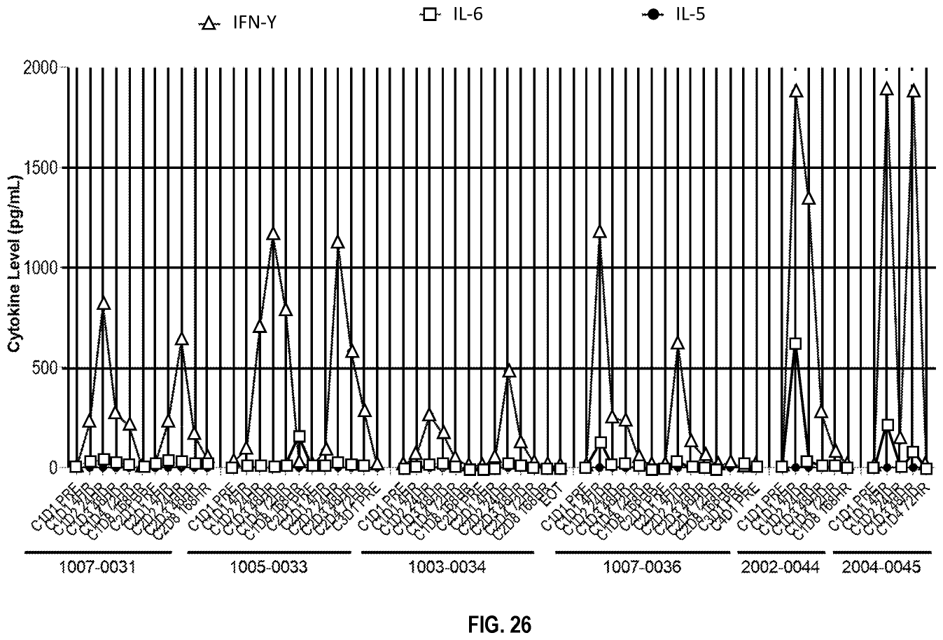

Levels of IFN-γ, IL-5, and IL-6 were also measured (FIG. 26). The measured values show that IFN-γ was induced, but low amounts of IL-5 and IL-6, cytokines associated with VLS and CRS, respectively, were induced, except for one subject in whom IL-6 levels increased to about 700 μg/mL at 4 hours after treatment but decreased thereafter.

Summary of Results; Discussion. All subjects tested had post-dose peripheral expansion of CD8+ T effector (Teff) cells, CD8+ memory cells, NK cells, and CD4+ T_{reg} cells. There were no meaningful elevations in IL-5 levels and the subject whose IL-6 level was increased at day 3 showed a reduction the following day.

One subject experienced fever at hour 16 on day one of the first cycle and at hour 9 on the first day of the second cycle. A second subject had an elevated blood pressure (162/9 mm Hg) 16 hours after dosing in the first cycle. A third subject experienced two infusion reactions. The first was a grade 1 response 2.5 hours post dose on day one of the first cycle. The second was a grade 3 response 4 hours post dose on day one of the second cycle. On day one of the first cycle, a fourth subject experienced a grade one CRS that included fever, rigors and decreased blood pressure (135/63 to 106/61 mm Hg). A fifth patient experienced G2 CRS consisting of fever and hypotension that was managed with hydration and dexamethasone. Subsequently developed G3 transaminitis treated with dexamethasone. On C2D1 subject experienced a second episode of G2CRS managed with steroids and hydration.

In summary, all 6 subjects had at least one treatment-emergent AE (TEAE). TEAEs are detailed in Table 6. No TEAEs were grade 4 or 5.

TABLE 6
Treatment Emergent Adverse Events (TEAE): n = 6

| System Organ Class | Grade 1 | | Grade 2 | | Grade 3 | | Grade 4 | | Grade 5 | |
|---|---|---|---|---|---|---|---|---|---|---|
| General disorders and | 3/6 | (50%) | 2/6 | (33.3%) | 0/6 | (0% | 0/6 | (0%) | 0/6 | (0%) |
| administration site conditions | | | | | | | | | | |
| Gastrointestinal disorders | 3/6 | (50%) | 2/6 | (33.3%) | 0/6 | (0%) | 0/6 | (0%) | 0/6 | (0%) |
| Investigations | 1/6 | (16.7%) | 1/6 | (16.7%) | 2/6 | (33.3%) | 0/6 | (0%) | 0/6 | (0%) |
| Immune System Disorders | 0/6 | (0%) | 2/6 | (33.3%) | 0/6 | (0%) | 0/6 | (0%) | 0/6 | (0%) |
| Infections and infestations | 0/6 | (0%) | 0/6 | (0%) | 1/6 | (16.7%) | 0/6 | (0%) | 0/6 | (0%) |
| Injury, Procedural | 0/6 | (0%) | 0/6 | (0%) | 1/6 | (16.7%) | 0/6 | (0%) | 0/6 | (0%) |
| Complications | | | | | | | | | | |
| Nervous system disorders | 2/6 | (33.3%) | 1/6 | (16.7%) | 0/6 | (0%) | 0/6 | (0%) | 0/6 | (0%) |
| Skin and subcutaneous tissue | 1/6 | (16.7%) | 0/6 | (0%) | 0/6 | (0%) | 0/6 | (0%) | 0/6 | (0%) |
| disorders | | | | | | | | | | |
| Metabolism and nutrition | 1/6 | (16.7%) | 1/6 | (16.7%) | 2/6 | (33.3%) | 0/6 | (0%) | 0/6 | (0%) |
| disorders | | | | | | | | | | |
| Respiratory, thoracic and | 1/6 | (16.7%) | 2/6 | (33.3%) | 0/6 | (0%) | 0/6 | (0%) | 0/6 | (0%) |
| mediastinal disorders | | | | | | | | | | |
| Musculoskeletal and | 4/6 | (66.7%) | 0/6 | (0%) | 0/6 | (0%) | 0/6 | (0%) | 0/6 | (0%) |
| connective tissue disorders | | | | | | | | | | |
| Psychiatric disorders | 1/6 | (16.7%) | 0/6 | (0%) | 0/6 | (0%) | 0/6 | (0%) | 0/6 | (0%) |
| Vascular Disorders | 0/6 | (0%) | 2/6 | (33.3%) | 0/6 | (0%) | 0/6 | (0%) | 0/6 | (0%) |

Two patients had SAEs: One patient had a grade 1 CRS (fever, chills and BP decrease from 135/62 to 106/61 mm Hg) that required hospitalization but was managed with hydration and electrolyte replacement. Another patient had grade 3 transaminitis on the first cycle and grade 2 CRSs on both cycles 1 and 2 (fever and hypotension) that was managed with dexamethasone.

One DLT, an infusion related reaction requiring a dosage reduction, occurred. While there were no drug discontinuations resulting from TEAE, one patient dosage reduction occurred as a result of TEAE (G3 infusion reaction). No subjects experienced anaphylaxis.

Treatment-related AEs resolved with accepted standard of care. There were no meaningful elevations in IL-5, no cumulative toxicity, no end organ toxicity, and no QTc prolongation or other cardiac toxicity associated with G3 hypertension and G4 lymphopenia. Accordingly, the IL-2 conjugate demonstrated encouraging PD data and was generally well-tolerated. PK data (Table 7) were consistent with an in vivo half-life of the IL-2 conjugate of about 10 hours. Overall, the results are considered to support non-alpha preferential activity of the IL-2 conjugate, with a tolerable safety profile, encouraging PD and preliminary evidence of activity in patients with immune-sensitive tumors.

For PK assessments, samples were taken from the subjects to determine the concentration of drug in the blood over time. Table 7 reports the mean and standard deviation of blood concentration levels measured in cycles 1 and 2.

TABLE 7
Concentration versus Time summary

| | Nominal | | | |
|---|---|---|---|---|
| cycle | Time (hr) | N | Mean | Std Deviation |
| 1 | 0.0 | 0 | BLQ | N/A |
| | 0.5 | 6 | 561 | 237 |
| | 1.0 | 6 | 547 | 230 |
| | 2.0 | 6 | 516 | 162 |
| | 4.0 | 6 | 405 | 154 |
| | 8.0 | 6 | 303 | 95.8 |
| | 12.0 | 6 | 259 | 38.0 |
| | 24.0 | 6 | 168 | 34.0 |
| | 48.0 | 6 | 64.1 | 29.2 |
| | 72.0 | 6 | 16.7 | 14.4 |
| | 168.0 | 1 | 1.01 | N/A |
| 2 | 0.0 | 0 | BLQ | N/A |
| | 0.5 | 4 | 338 | 180 |
| | 1.0 | 4 | 376 | 234 |
| | 2.0 | 4 | 336 | 156 |
| | 4.0 | 4 | 280 | 88.6 |
| | 8.0 | 4 | 219 | 62.1 |
| | 12.0 | 3 | 180 | 35.2 |
| | 24.0 | 4 | 115 | 37.3 |
| | 48.0 | 3 | 24.2 | 7.05 |
| | 72.0 | 2 | 2.97 | N/A |

### Example 4. Clinical Study of Diffuse Large B-cell Lymphoma Treatment Using an IL-2 Conjugate

A Phase 2 non-randomized, open-label, multi-center study assessing the clinical benefit of the IL-2 conjugate described in Example 1, as at least a third line of therapy, for the treatment of participants with diffuse large B-cell lymphoma (DLBCL) is undertaken. The participants are patients aged 12 years and older with DLBCL, and who must have received at least two lines of systemic therapy and are post-chimeric antigen receptor T-cell (CAR-T) treatment.

The participants will receive the IL-2 conjugate every 3 weeks on day 1 of each cycle (21 days per cycle), for up to 35 cycles.

The following inclusion criteria apply. The participant must be >12 years of age, at the time of signing the informed consent. The participant's disease location must be amenable to tumor biopsy at baseline. The participant must have a measurable disease. The participant, if female, is eligible to participate if she is not pregnant or breastfeeding, is not a woman of childbearing potential (WOCBP) or is a WOCBP who agrees: (1) to use approved contraception method and submit to regular pregnancy testing prior to treatment and for at least 180 days after discontinuing study treatment, and (2) to refrain from donating or cryopreserving eggs for 180 days after discontinuing study treatment. The participant, if male, is eligible to participate if he agrees to refrain from donating or cryopreserving sperm, and either abstain from heterosexual intercourse or use approved contraception during study treatment and for at least 210 days after discontinuing study treatment. The participant must be capable of giving signed informed consent. The participant must have histologically or cytologically confirmed diagnosis of DLBCL, must have received at least two prior lines of systemic therapy for DLBCL, including at least one containing a combination of an anthracycline and rituximab (or another anti-CD20 agent), with the last line of therapy a marketed anti-CD19 CAR-T therapy.

Participants are excluded from the study if any of the following criteria applies:
Eastern Cooperative Oncology Group (ECOG) performance status of ≥2 (≥16 years old)
Lansky Scale (<16 years old)<50%.
Poor bone marrow reserve
Poor organ function
Participants with baseline SpO₂≤92%
Lymphomatous involvement of the central nervous system
History of allogenic or solid organ transplant
Last administration of prior antitumor therapy or any investigational treatment within 21 days or less than 5 times the half-life, whichever is shorter; major surgery or local intervention within 21 days
Received prior IL-2-based anticancer treatment
Comorbidity requiring corticosteroid therapy
Antibiotic use (excluding topical antibiotics)≤14 days prior to first dose of IL-2 conjugate
Severe or unstable cardiac condition within 6 months prior to starting study treatment
Active, known, or suspected autoimmune disease that has required systemic treatment in the past 2 years
Known second malignancy either progressing or requiring active treatment within the last 3 years
Receipt of a live or live attenuated virus vaccination within 28 days of planned treatment start (seasonal flu vaccines or SARS-CoV-2 vaccine that do not contain live virus are permitted).

The progression of disease can be monitored in patients according to various criteria.

Complete response rate (CRR), defined as the proportion of participants who have a complete response (CR) determined, determined per the Lugano response criteria 2014, can be evaluated. CRR can be evaluated up to the date of the first documented progression or initiation of subsequent anticancer therapy or approximately 8 months after the last participant receives their first dose.

Objective response rate (ORR) can be evaluated as the proportion of participants who have CR or partial response (PR), determined per the Lugano response criteria 2014. ORR can be evaluated up to the date of first documented progression or initiation of subsequent anticancer therapy, or approximately 8 months after the last participant receives their first dose.

Time to response, defined as the time from the first administration of the IL-2 conjugate to the first documented evidence of PR or CR, determined per the Lugano response criteria 2014, can be evaluated. Time to response can be evaluated from the date of first dose until the date of first documented progression or date of death from any cause, whichever comes first, assessed up to 36 months.

Duration of response (DoR), defined as the time from first documented evidence of CR or PR until progressive disease (PD), determined per the Lugano response criteria 2014, or death from any cause, whichever occurs first, can be evaluated. DoR can be evaluated from the date of first dose until the date of first documented progression or date of death from any cause, whichever comes first, assessed up to 36 months.

Clinical benefit rate (CBR), including CR or PR at any time or stable disease (SD) of at least 6 months from the first administration of the IL-2 conjugate until PD (per the Lugano response criteria 2014), or death from any cause, whichever occurs first, can be evaluated. CBR can be evaluated up to the date of first documented progression or initiation of subsequent anticancer therapy or approximately 8 months after the last participant receives their first dose.

Progression free survival (PFS), defined as the time from the date of first the IL-2 conjugate administration to the date of the first documented disease progression as per the Lugano response criteria 2014, or death due to any cause, whichever occurs first, can be evaluated. PFS can be evaluated from the date of first dose until the date of first documented progression or date of death from any cause, whichever comes first, assessed up to 36 months.

CAR-T cell activation and proliferation, along with suppression of circulating B cells, can be evaluated up to the date of the first documented progression or initiation of subsequent anticancer therapy or approximately 8 months after the last participant receives their first dose.

Pharmacokinetic parameters, such as the concentration of the IL-2 conjugate, and the incidence of any anti-drug antibodies (ADAs) against the IL-2 conjugate, can also be evaluated in patients at various time points throughout the study. For example, the plasma concentration of the IL-2 conjugate can be evaluated at day 1 and day 15 of Cycle 1, at day 1 of cycle 2-4-7-10, plus every fifth cycle (each cycle is 21 days), for a maximum of up to approximately 24 months. Also, the incidence of any ADAs against the IL-2 conjugate can be evaluated at day 1 and day 15 of Cycle 1, at day 1 of Cycle 2-4-7-10, plus every fifth cycle (each cycle is 21 days) and 30 days after the last IL-2 conjugate administration, for a maximum of up to approximately 24 months.

To confirm the dose of the IL-2 conjugate when combined with or without other anticancer therapies, the incidence of any dose-limiting toxicities (DLTs) can be evaluated for one cycle (21 days). DLTs include, for example, Grade 3 neutropenic fever (absolute neutrophil count (ANC)<1,000/mm³ with single temperature >38.3° C. (101° F.) or sustained temperature >38° C. (100.4° F.) for more than 1 hour).

To assess the safety profile of the IL-2 conjugate when combined with or without other anticancer therapies, the incidence of any treatment-emergent adverse events (TEAEs) and laboratory abnormalities can be evaluated according to the National Cancer Institute Common Terminology Criteria for Adverse Events (NCI CTCAE) V5.0 and the American Society for Transplantation and Cellular Therapy (ASTCT) consensus gradings. Any such TEAEs and laboratory abnormalities can be evaluated from the first IL-2 conjugate dose up to 30 days after the last IL-2 conjugate dose.

To assess the safety profile of the IL-2 conjugate when combined with or without other anticancer therapies, the incidence of any serious adverse events (SAEs) and laboratory abnormalities can be evaluated according to the National Cancer Institute Common Terminology Criteria for Adverse Events (NCI CTCAE) V5.0 and the American Society for Transplantation and Cellular Therapy (ASTCT) consensus gradings. Any such SAEs and laboratory abnormalities can be evaluated from the first IL-2 conjugate dose up to 90 days after the last IL-2 conjugate dose.

The duration of the study period per participant is up to 6 years (screening period [28 days], treatment period [max 35 cycles=735 days], and follow-up period [approximately 3 years]).

### Example 5. Use of an IL-2 Conjugate to Stimulate Proliferation and Persistence of CAR-T Cells in Co-culture with Large B Cell Lymphoma (LBCL) Cells

Using three patient peripheral blood mononuclear cell samples to generate CAR-T cells, a study was conducted to evaluate the addition of the IL-2 conjugate described in Example 1 to the CAR-T cells in co-culture with two wild-type LBCL cell lines (HT and RL). The CAR-T cells expressed a construct equivalent to the CD3/CD28 design used in the anti-CD19 CAR-T cell axicabtagene ciloleucel. A coculture was created at a 1:1 effector-to-target ratio, and fresh cancer cells were added every second day with the IL-2 conjugate or vehicle control, up until day 6. Flow cytometry analysis was performed on days 4 and 8 to determine cell counts as well as immunophenotyping of the T cells.

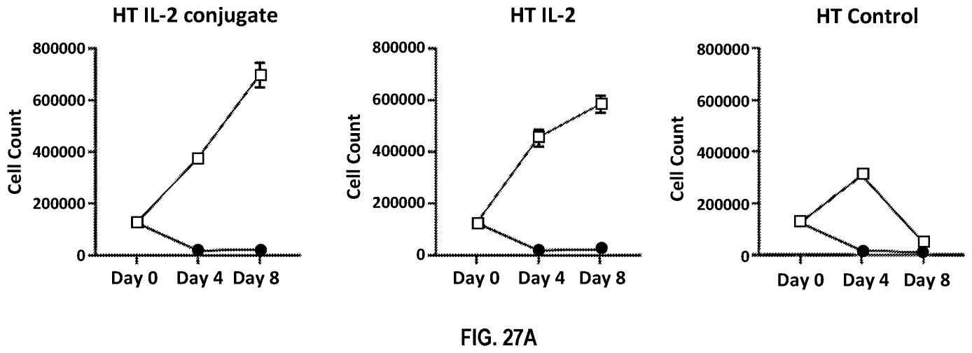

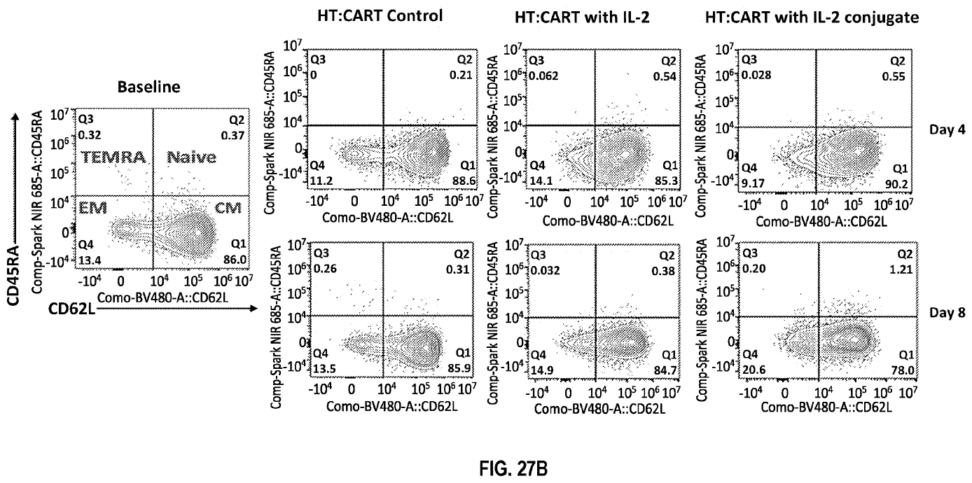

Addition of the IL-2 conjugate significantly increased the expansion CAR-T cells, increased the expression of activation markers, and reduced the expression of exhaustion markers. The IL-2 conjugate led to sustained proliferation of CAR-T cells for at least 8 days, as shown in FIG. 27A. This effect was more robust when compared to the control condition (FIG. 27A, right) and well as the condition in which IL-2 was added (FIG. 27A, center). Moreover, addition of the IL-2 conjugate led to an increase in the proportion of effector memory (EM) cells at day 8. See FIG. 27B, Q4 of each part. Therefore, the IL-2 conjugate can inhibit terminal differentiation of CAR-T cells, as evidenced by the sustained proliferation of CAR-T cells and the increase in effector memory cell generation, contributing to sustained killing of LBCL cells.

### Example 6. Evaluation of Concentration of an IL-2 Conjugate in Combination with Anti-CD19/CD22 CAR-T Cells in a Lymphoma Mouse Model

### Materials and Methods

A study was conducted to evaluate the concentration of the IL-2 conjugate of Example 1 for use with anti-CD19/CD22 CAR-T cells, against Raji-Luc Burkitt's lymphoma cells in MHC I/II double knockout NOD Cg-Prkdc^{scid} H2-K 1^{tm1Bpe} H2-Abl^{emlMvw} H2-D1^{tm1Bpe} Il2rg^{tm1Wjl}/SzJ (NSG-MHC I/II DKO) mice (The Jackson Laboratory, Bar Harbor, Maine).

The mice were treated and evaluated as described in Table 8. On Day 0, the mice were engrafted with 0.5×10⁶ Raji-Luc cells in a 200 μL dose. The mice were enrolled in the four groups shown in Table 8. On Days 9, 16, and 23, the IL-2 conjugate was intravenously injected according to Table 8. On Day 10, the mice intravenously received 2×10⁶ anti-CD19/CD22 CAR-T cells (ProMab Biotechnologies, Richmond, California). The anti-CD19/CD22 CAR of the CAR-T cells comprised the sequence of SEQ ID NO: 43.

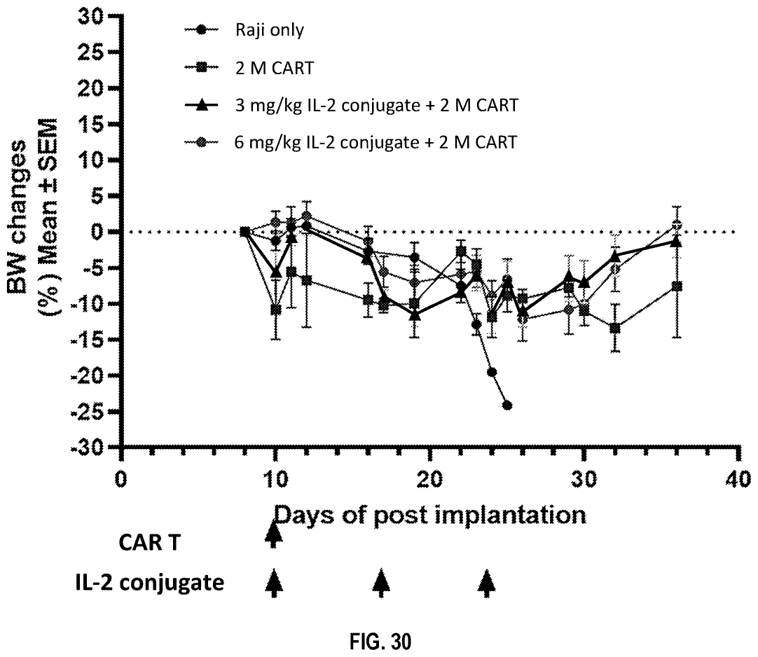

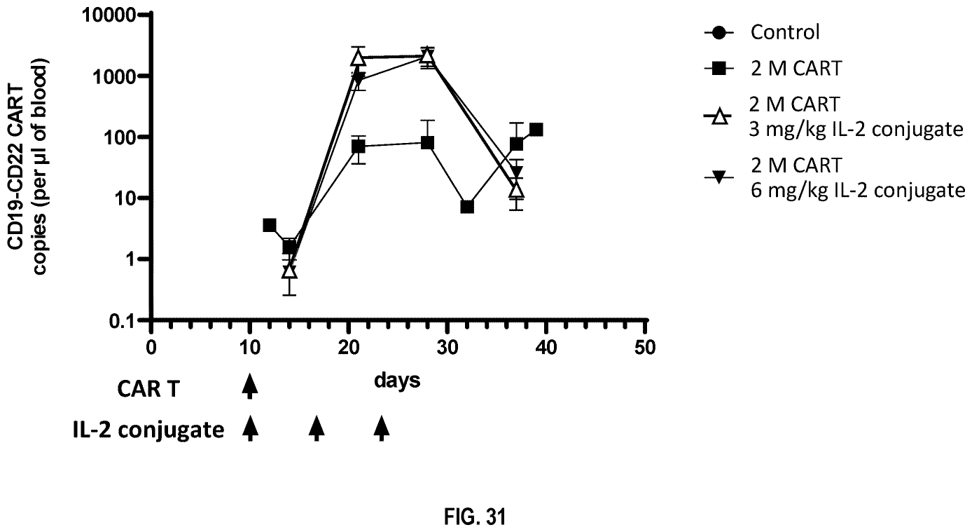

The mice were imaged on Days 7, 11, 15, 22, 29, and 36. Prior to imaging, the mice were intraperitoneally injected with 150 mg/kg D-luciferin, at a dose volume of 10 mL/kg. The mice were weighed on select days, as shown in FIG. 30. For droplet digital polymerase chain reaction (ddPCR) analysis on select days, as shown in FIG. 31, 50 μL whole blood was drawn from the mice's mandibular veins into K2EDTA tubes on wet ice, and then stored at −80° C.

TABLE 8
Treatment and Evaluation of NSG Mice Injected with Raji-Luc Cells

| | 7W Female | | | IL-2 | | Blood |
|---|---|---|---|---|---|---|
| | NSG, DKO | Raji-Luc, | Treatment | Conjugate, IV, | Imaging | Collection Days |
| Group | (N) | IV, D 0 | IV, D 10 | Starting D 9 | Days | for ddPCR |
| 1 | 4 | 0.5M | — | — | D 7, D 11, | — |
| | | | | | D 15, D 22, | |
| | | | | | D 29, D 36 | |
| 2 | 4 | 0.5M | 2M, CAR-T, | Vehicle | D 7, D 11, | At least |
| | | | one dose | (saline) | D 15, D 22, | D 14, D 21, |
| | | | | Q1W × 3 | D 29, D 36 | D 28 |
| 3 | 4 | 0.5M | 2M, CAR-T, | 3 mg/kg, | D 7, D 11, | At least |
| | | | one dose | Q1W × 3 | D 15, D 22, | D 14, D 21, |
| | | | | | D 29, D 36 | D 28 |
| 4 | 4 | 0.5M | 2M, CAR-T, | 6 mg/kg, | D 7, D 11, | At least |
| | | | one dose | Q1W × 3 | D 15, D 22, | D 14, D 21, |
| | | | | | D 29, D 36 | D 28 |

### Results

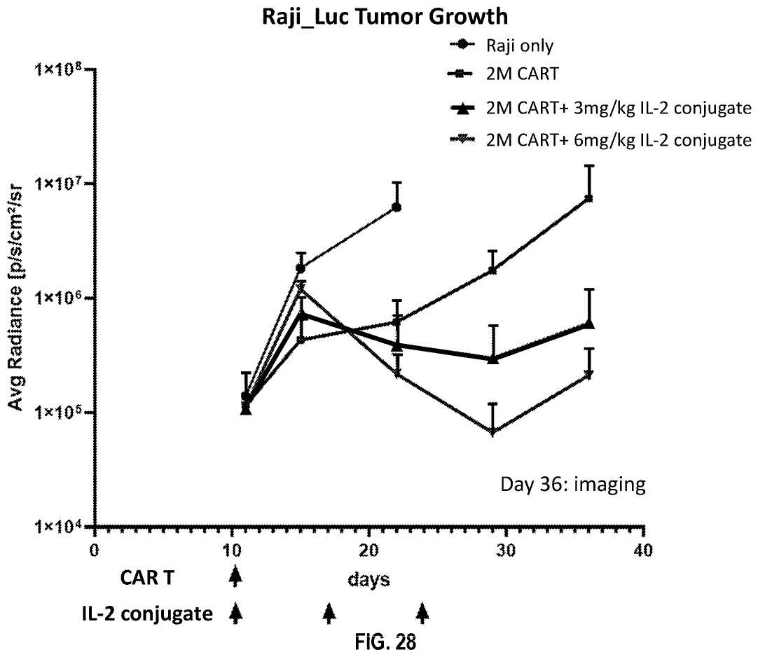

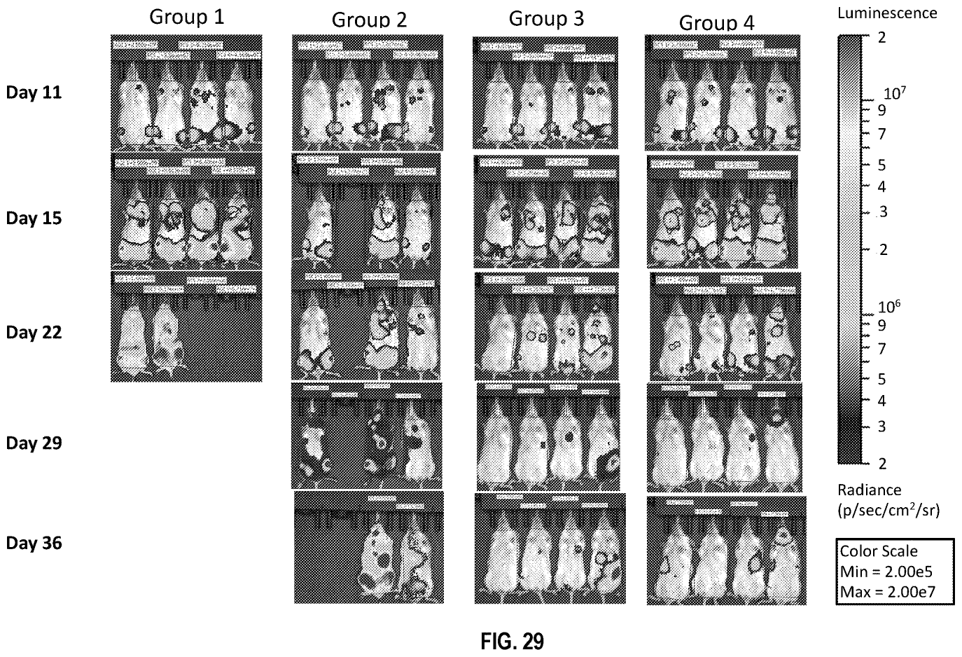

FIG. 28 shows average radiance, which reflects the growth of the engrafted Raji-Luc cells, in the mice in the four groups. FIG. 29 shows imaging of the radiance in the mice in the four groups. The addition of the CAR-T cells in Group 2 resulted in suppression of Raji-Luc cell growth, and the addition of both the CAR-T cells and the IL-2 conjugate in Groups 3 and 4 resulted in even greater suppression of Raji-Luc cell growth. FIG. 30 shows body weight changes of the mice in the four groups.

FIG. 31 shows copy numbers of the anti-CD19/CD22 CAR-T construct, as determined by ddPCR. Group 1 (Control”) has numbers around 0 and are not evident in this figure (since the mice in Group 1 did not receive the anti-CD19/CD22 CAR-T cells).

### Example 7. Evaluation of Antitumor Activity of an IL-2 Conjugate and Anti-CD19/CD22 CAR-T Cells, as Single Agents and in Combination, in a Lymphoma Mouse Model

### Materials and Methods

A study was conducted to evaluate the antitumor activity of the IL-2 conjugate of Example 1 and anti-CD19/CD22 CAR-T cells, as single agents and in combination, against Raji-Luc Burkitt's lymphoma cells in MHC I/II double knockout NOD.Cg-Prkdc^{scid} H2-KI^{tmlBpe} H2-Ab1^{emkMvw} H2-D1^{tmlBpe} Il2rg^{tmlWjl}/SzJ (NSG-MHC I/II DKO) mice (The Jackson Laboratory, Bar Harbor, Maine).

The mice were treated and evaluated as described in Table 9. On Day 0, 40 mice were weighed and were then engrafted with 0.5×10⁶ Raji-Luc cells in a 200 μL dose having a concentration of 2.5×10⁶ cells/mL. On Day 7, the mice were weighed. On Day 8, the mice were enrolled in the five groups shown in Table 9. On Days 9, 16, and 23, the IL-2 conjugate was intravenously injected according to Table 9, and the mice were monitored for the first one hour and at six hours post-dosing, and the mice were also weighed. On Day 10, the mice were weighed before intravenously receiving 2×10⁶ untransduced T (mock-T) cells or anti-CD19/CD22 CAR-T cells (ProMab Biotechnologies, Richmond, California). The anti-CD19/CD22 CAR of the CAR-T cells comprised the sequence of SEQ ID NO: 43. The mice were monitored for the first one hour and at six hours post-dosing.

The mice were imaged on Days 7, 11, 18, 23, 25, and 29. Prior to imaging, the mice were intraperitoneally injected with 150 mg/kg D-luciferin, at a dose volume of 10 mL/kg. After Day 21, the mice were monitored daily, and their death dates and phenotypes were recorded.

On Day 14, for ddPCR analysis, 50 μL whole blood was drawn from the mice's mandibular veins into K2EDTA tubes on wet ice, and then stored at −80° C. On Day 21, for flow cytometry and ddPCR analyses, 100 μL whole blood was drawn from the mice's mandibular veins into K2EDTA tubes, aliquoted into 50 μL portions, and then stored at −80° C. On Days 22 and 25, for ddPCR analysis of select groups, 50 μL whole blood was drawn from the mice's mandibular veins into K2EDTA tubes on wet ice, and then stored at −80° C. On Day 28, for flow cytometry and ddPCR analyses, 100 μL whole blood was drawn from the mice's mandibular veins into K2EDTA tubes, aliquoted into 50 μL portions, and then stored at −80° C.

The mice received at least twice-per-week body weight (BW) measurements and were clinically observed. The mice were euthanized if BW loss exceeded 20% or developed hind-leg paralysis. By Day 21, in each of Groups 1, 2, and 4, one mouse had been euthanized. By Day 22, in each of Groups 1 and 2, one mouse had been euthanized; in Group 4, two mice had been euthanized. By Day 25, in each of Groups 1, 2, 3, and 5, one mouse had been euthanized; in Group 4, two mice had been euthanized. By Day 28, in each of Groups 1, 2, and 4, all mice had been euthanized; in Group 3, four mice had been euthanized; in Group 5, three mice had euthanized. By Day 31, in each of Groups 1, 2, and 4, all mice had been euthanized; in Group 3, four mice had been euthanized; in Group 5, three mice had euthanized.

When the mice were euthanized early or eventually, on Day 31, 100 μL whole blood was drawn for ddPCR analysis and stored at −80° C., and the remainder (>200 μL) of whole blood was drawn and processed for cytokine analysis. The mice's livers and spleens were weighed before being fixed for immunohistochemistry analysis.

TABLE 9
Treatment and Evaluation of NSG Mice Injected with Raji-Luc Cells

| | 7W Female | | | IL-2 | | Blood |
|---|---|---|---|---|---|---|
| | NSG, DKO | Raji-Luc, | Treatment, | Conjugate, IV, | | Collection Days |
| Group | (N) | IV, D 0 | IV, D 10 | Starting D 9 | Imaging Days | for ddPCR |
| 1 | 8 | 0.5M | — | — | D 7, D 11, D 18, | D 14, D 21 (FC), |
| | | | | | D 23, D 25, D 29 | D 28 (FC), D 31 |
| 2 | 8 | 0.5M | 2M mock-T, | Vehicle | D 7, D 11, D 18, | D 14, D 21 (FC), |
| | | | one dose | (saline) | D 23, D 25, D 29 | D 28 (FC), D 31 |
| | | | | Q1W × 3 | | |
| 3 | 8 | 0.5M | 2M CAR-T, | Vehicle | D 7, D 11, D 18, | D 14, D 21 (FC), |
| | | | one dose | (saline) | D 23, D 25, D 29 | D 28 (FC), D 31 |
| | | | | Q1W × 3 | | |
| 4 | 8 | 0.5M | 2M mock-T, | 6 mg/kg, | D 7, D 11, D 18, | D 14, D 21 (FC), |
| | | | one dose | Q1W × 3 | D 23, D 25, D 29 | D 28 (FC), D 31 |
| 5 | 8 | 0.5M | 2M CAR-T, | 6 mg/kg, | D 7, D 11, D 18, | D 14, D 21 (FC), |
| | | | one dose | Q1W × 3 | D 23, D 25, D 29 | D 28 (FC), D 31 |

* FC = flow cytometry

### Results

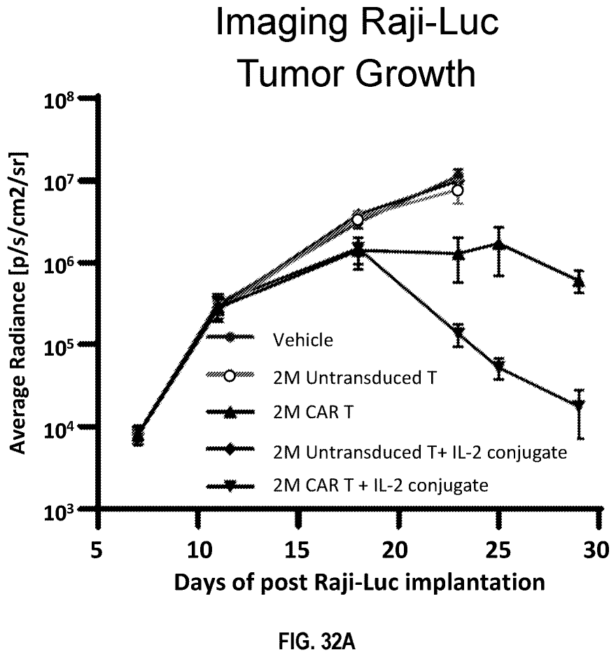

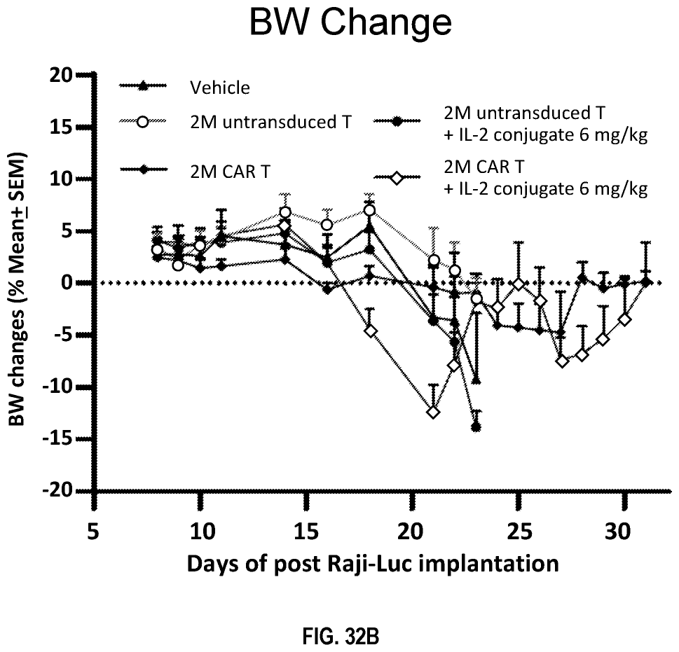

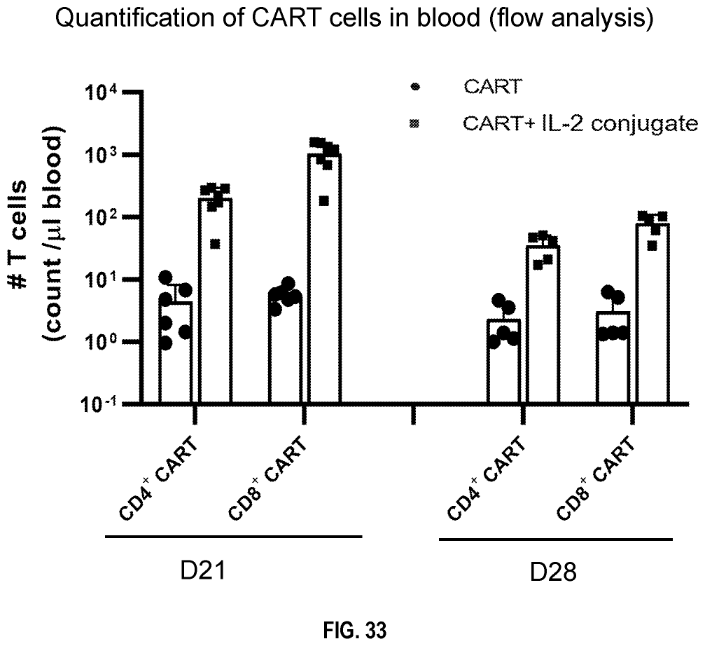

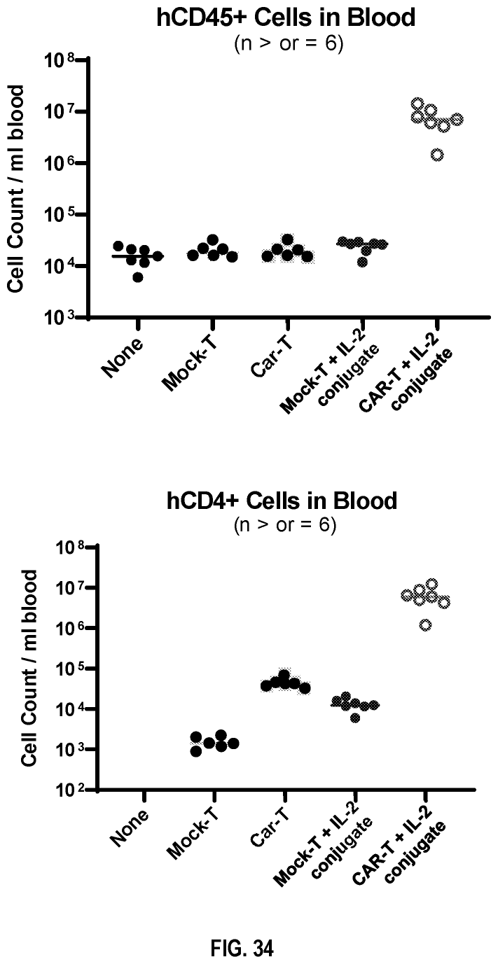

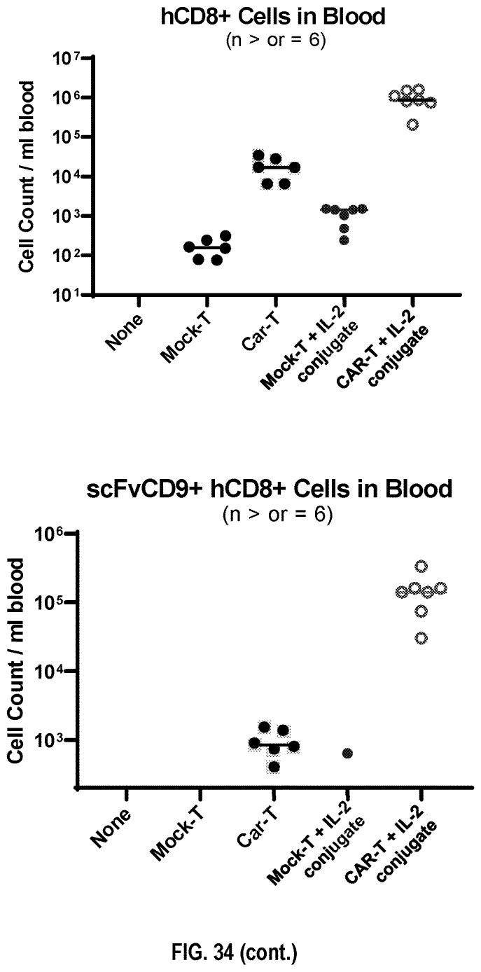

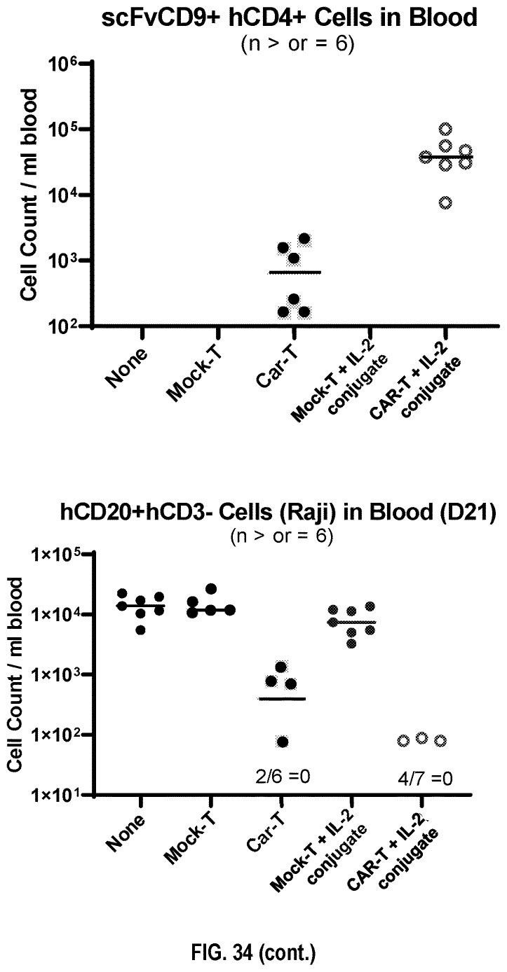

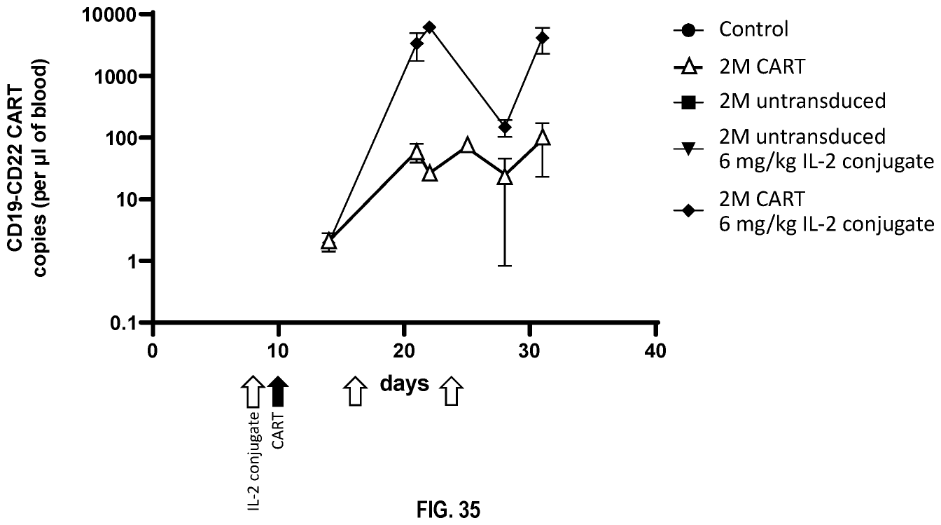

FIG. 32A shows average radiance, which reflects the growth of the engrafted Raji-Luc cells, in the mice in the five groups. The addition of the CAR-T cells in Group 3 resulted in suppression of Raji-Luc cell growth, and the addition of both the CAR-T cells and the IL-2 conjugate in Group 5 resulted in even greater suppression of Raji-Luc cell growth. FIG. 32B shows body weight changes of the mice in the five groups.

FIG. 33 shows the quantities of CD4⁺ CAR-T cells and CD8⁺ CAR-T cells for Groups 3 and 5 on Days 21 and 28, as quantified by flow cytometry. The addition of the IL-2 conjugate significantly increased the numbers of both CD4⁺ CAR-T cells and CD8⁺ CAR-T on Days 21 and 28.

FIG. 34 shows the the quantities of different types of cells on Day 21, as quantified by flow cytometry. The addition of both the CAR-T cells and the IL-2 conjugate in Group 5 significantly increased the numbers of hCD45⁺ cells, hCD4⁺ cells, hCD8⁺ cells, scFvCD9⁺hCD8⁺ cells, and scFvCD9⁺ hCD4⁺ cells, and significantly decreased the number of Raji_Luc cells, compared to any one of Groups 1-4.

FIG. 35 shows copy numbers of the anti-CD19/CD22 CAR-T construct, as determined by ddPCR. The addition of the IL-2 conjugate in Group 5 significantly increased the copy number of the CAR-T construct, as compared to Group 3. Groups 1, 2, and 4 have copies numbers around 0 and are not evident in this figure (since the mice in Groups 1, 2, and 4 did not receive the anti-CD19/CD22 CAR-T cells).

### Example 8. Use of an IL-2 Conjugate to Improve Killing of BL-ALL Cells by Anti-CD19 CAR-NK Cells

### Materials and Methods

Lentivirus Preparation: One day prior to transfection, 5×10⁶ HEK293T cells were seeded in a 10 cm dish in Dulbecco's Modified Eagle Medium (DMEM) and 10% fetal bovine serum (FBS) and incubated overnight, so that cells were approximately 80% confluent the next day

The next day, a plasmid DNA mix was prepared by adding 1 μg pUC.BaEV-Rless plasmid, 6.54 μg Gagpol plasmid, 3.27 μg REV plasmid, and 18 μg CAR19 gene transfer plasmid encoding the CAR for axicabtagene ciloleucel into 2 mL of serum-free medium in a 50 mL tube, which was mixed by vortexing.

30 μL of Lipofectamine 2000 was added to 2 mL of serum-free medium and mixed by vortexing. The diluted Lipofectamine 2000 mix was then added to the DNA mix and vortexed before incubating at room temperature for 20 minutes. 5 mL DMEM and 10% FBS was then added and mixed gently by inverting the tube a few times.

The dish containing the HEK293T cells was removed from the incubator and examined under a microscope, and the medium was carefully aspirated before the transfection mixture was carefully added without disrupting the monolayer of cells. The plate was then incubated at 37° C. overnight. Then, the transfection medium was exchanged for fresh DMEM and 10% FBS and incubated for 48 hours before the supernatant containing virus was harvested. The supernatant was then filtered through 0.45 μm polyvinylidene fluoride (PVDF) filters. One volume Lenti-X™ Concentrator (Takara, Cat. No. 631231) was mixed with three volumes medium containing virus and left overnight in the refrigerator. The Lenti-X and virus mixture was then centrifuged at 600 g for 45 minutes, the supernatant was discarded, and the pellet was resuspended in 0.5 mL phosphate-buffered saline (PBS). Lenti-X™ GoStix™ (Takara, Cat. No. 631280) was used to verify the presence of virus. Aliquots of virus were stored at −80° C.

NK Cell Isolation and Transduction: NK cells were isolated from frozen peripheral blood mononuclear cells (PBMCs) from two separate donors using Stemcell isolation reagents (Cat. Nos. 17955, 20144) and resuspended at 0.5×10⁶ cells/mL. 1.5 mL were plated per well in a 12-well plate. IL-2 and PM21, particles generated from K562-derivative cells expressing IL-21 and 4-1BB, were added, and the cells were incubated for seven days. The cells were then washed and counted and resuspended at 1.25×10⁶ cells/mL in CTS Xpander Media (Gibco, Cat. No. A49161-01) with 10% human AB serum and IL-2 (20 ng/ml).

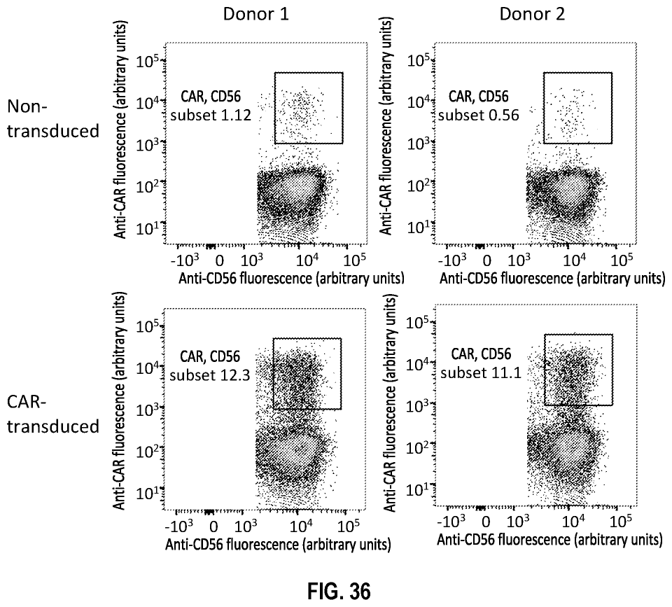

250,000 cells per well were seeded in 200 μL in a 96-well flat-bottom plate with 10 μg/mL of Vectofusin (Miltenyi, Cat. No. 130-111-163) and thawed virus. Non-transduced control was included. The plate was centrifuged at room temperature for 45 minutes at 600 g in a Beckman Coulter benchtop centrifuge. The plate was then incubated for 72 hours. Then, the cells were harvested and washed, and an aliquot was taken to verify CAR expression using flow cytometry. Briefly, the cells were resuspended in staining buffer with TruStain FcX Fc block (BioLegend, Cat. No. 422302), anti-CD56 BV421 (BioLegend, Cat. No. 318328), and CD19 Fc chimera Atto647 (R & D Biosystems, Cat. No. DMUL0522101) for 20 minutes at 4° C. The cells were then washed twice in staining buffer before being analyzed on a BD Fortessa flow cytometer. The percentage cells expressing CAR was determined as CD56+ and CAR+ (FIG. 36). FlowJo was used for analysis of flow cytometry data.

Labeling of Nalm6 Target Cells: The CD19-expressing B-lineage acute lymphoblastic leukemia (BL-ALL) cell line Nalm6 was chosen as a target cell line for cytotoxicity experiments with NK cells expanded in the presence of PM21 and expressing the CAR (CAR-NK cells). Sensitivity to puromycin in Nalm6 cells was determined by incubating them in the presence of several concentrations of puromycin (ranging from 10 μg/mL to 0.5 μg/mL) in RPMI 1640 and 10% FBS culture medium, and by observing cell death using a microscope. 1 μg/mL was determined to be a suitable concentration for selection. Nalm6 cells were introduced to a 48-well plate in the presence of NucLight Red lentivirus (Sartorius, Cat. No. 4625), the plate was centrifuged at for 45 minutes at 600 g in a benchtop centrifuge before being incubated overnight at 37° C. The medium was exchanged, and cells were incubated for a further day before puromycin selection was performed. Once puromycin was added, the cells were incubated until only NucLight Red positive cells were surviving. Non-transduced Nalm6 cells were included as a control for selection. For maintenance in culture, the cells were cultured in the presence of 0.5 μg/mL puromycin.

CAR-NK Killing Assay: Duplicate wells were set up. A 96-well flat-bottomed plate (black with clear bottom) was coated with 50 μL poly-L ornithine and allowed to stand for 30 minutes in the hood. After 30 minutes, the solution was aspirated, and the plate was allowed to dry in the hood. Target Nalm6 cells expressing NucLight Red were plated at 15,000 cells/well in 100 μL volume in RPMI and 10% FBS medium in the plate.

The IL-2 conjugate of Example 1 was prepared in a 1 in 5 dilution series for final concentrations of 2 μg/mL, 0.4 μg/mL, 0.08 μg/mL, 0.016 μg/mL, and 0 μg/mL in PBS. (5 μL stock of the IL-2 conjugate at 2 mg/mL was added to 500 μL medium, and from there, 100 μL was transferred to 400 μL for a 1 in 5 dilution and repeated twice more, 0 g/mL was also included). 22 μL of the IL-2 conjugate was added to the target Nalm6 cells.

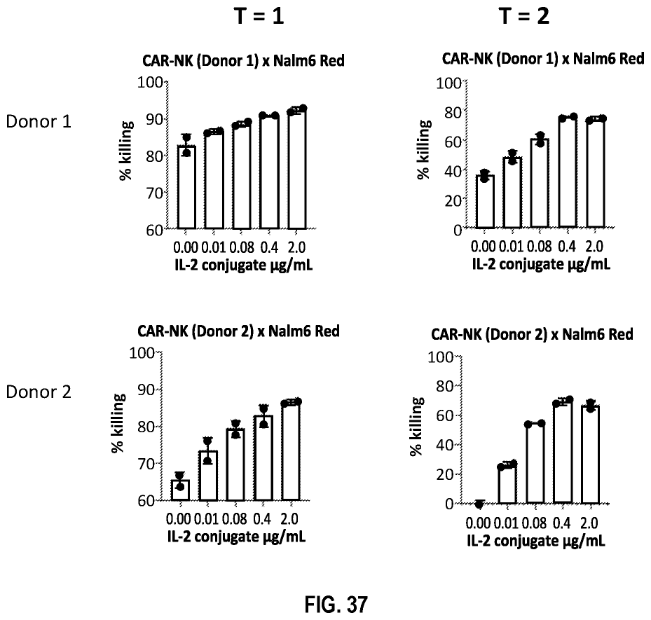

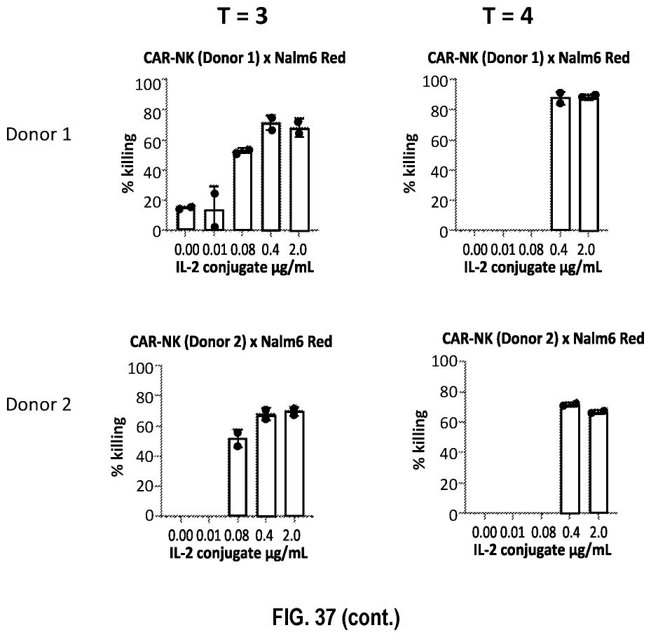

CAR-NK cells were added at 45,000 cells/well in 100 L for an effector-to-target ratio of 3:1, and the plate was centrifuged at 90 g for 10 minutes with deceleration set to 3. The plate was then placed in an Incucyte® S3 Live-Cell Imaging and Analysis System (Sartorius) and set to scan every 1-2 hours using the 4× objective to take one picture with the red fluorescent channel selected. Red object count was used to determine numbers of live cells. Counts obtained from wells containing target cells alone (no CAR-NK cells) were defined as 100% live cells or 0% killing and used to calculate % killing in experimental wells (FIG. 37).

Every 2-4 days, 100 μL supernatant was carefully removed without touching the bottom of the wells and was transferred to a new 96-well plate. The supernatant was then frozen for subsequent IFN-γ analysis. In the wells, the removed supernatant was replaced with 100 μL of fresh medium containing 15,000 Nalm6 NucLight Red cells, and the plate was centrifuged as before and returned to the Incucyte® System. (The first timepoint (T=1) was at 2 days, the second (T=2) was at 4 days, the third (T-3) was at 8 days, and the fourth (T=4) was at 10 days).

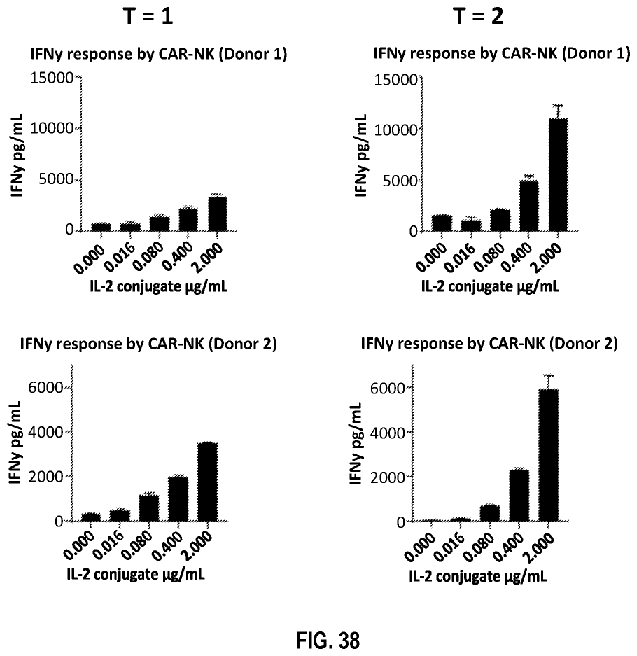

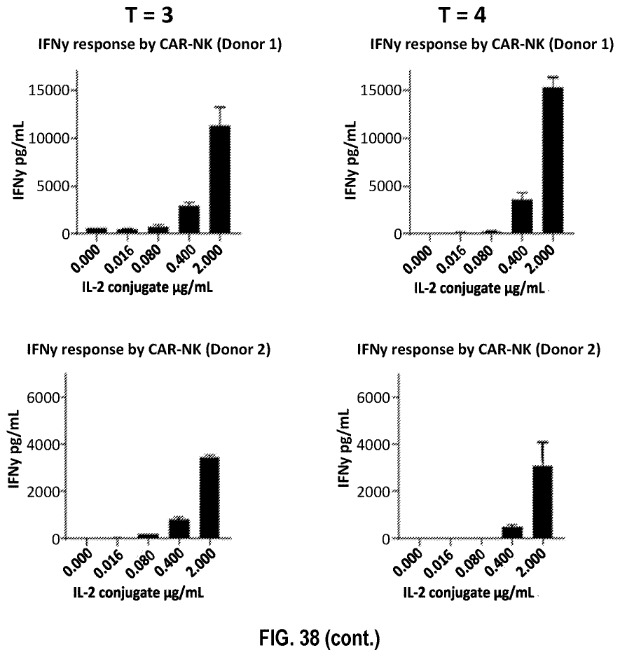

An IFN-γ detection kit (Mesoscale Discovery, Cat. No. K151QOD-4) and the Sector Imager 6000 (Mesoscale Discovery) was used to determine IFN-γ levels in the supernatants according to manufacturer's protocol (FIG. 38).

### Results

FIG. 36 shows CAR expression in NK cells from two donors. As shown in the figure, the CAR-NK cells generated from the NK cells from the two donors expressed CAR as described above at levels of 12% and 11%, respectively, and are shown in comparison to non-transduced control NK cells.

FIG. 37 shows Nalm6 cancer cell killing by CAR-NK cells measured at different time points (T=1, T=2, T=3, T=4) in the presence of an IL-2 conjugate at various concentrations. Specifically, the figure shows percentage killing of Nalm6 target cells by CAR-NK cells generated from the NK cells from two different donors at each indicated time point with or without an IL-2 conjugate, following each addition of Nalm6 cells. Killing of Nalm6 cells continues in an IL-2 conjugate dose dependent manner.

FIG. 38 shows IFN-γ production from co-cultures of CAR-NK cells and Nalm6 cells in the presence of an IL-2 conjugate. Specifically, the figure shows IFN-γ response from CAR-NK cells at each indicated time point, following each addition of Nalm6 cancer cells. Over time, IFN-γ is detected in the supernatants in a dose response manner to the IL-2 conjugate concentration for both donors.

As demonstrated, the presence of an IL-2 conjugate enhanced the ability and/or persistence of CAR-NK cells to kill Nalm6 target cells; such killing effect persisted over the course of more than 10 days, particularly when higher concentrations of an IL-2 conjugated were used. IFN-γ continued to be produced over time in a dose dependent manner, correlating with the IL-2 conjugate concentration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference.

### Table of Sequences

The following Table of Sequences provide the sequences referenced in the claims and specification.

| SEQ ID | | |
|---|---|---|
| NO | Description | Sequence |
| 1 | IL-2 | PTSSSTKKTQLQLEHLLLDLQMILNGINNY |
| | | KNPKLTRMLTFKFYMPKKATELKHLQCLE |
| | | EELKPLEEVLNLAQSKNFHLRPRDLISNIN |
| | | VIVLELKGSETTFMCEYADETATIVEFLNR |
| | | WITFSQSIISTLT |
| 2 | IL-2 conjugate | PTSSSTKKTQLQLEHLLLDLQMILNGINNY |
| | [AzK_L1_PEG30kD] is N6-((2- | KNPKLTRMLTFKFYMPKKATELKHLQCLE |
| | azidoethoxy)-carbonyl)-L-lysine | EELK[AzK_L1_PEG30kD]LEEVLNLAQSK |
| | stably-conjugated to PEG | NFHLRPRDLISNINVIVLELKGSETTFMCEY |
| | | ADETATIVEFLNRWITFSQSIISTLT |
| 3 | Anti-CD19 CDR-H1 | GVSLPDYG |
| | (in Examples 6 and 7) | |
| 4 | Anti-CD19 CDR-H2 | IWGSETT |
| | (in Examples 6 and 7) | |
| 5 | Anti-CD19 CDR-H3 | AKHYYYGGSYAMDY |
| | (in Examples 6 and 7) | |
| 6 | Anti-CD19 VH | EVKLQESGPGLVAPSQSLSVTCTVSGVSLP |
| | (in Examples 6 and 7) | DYGVSWIRQPPRKGLEWLGVIWGSETTYY |
| | | NSALKSRLTIIKDNSKSQVFLKMNSLQTDD |
| | | TAIYYCAKHYYYGGSYAMDYWGQGTSV |
| | | TVSS |
| 7 | Anti-CD19 CDR-L1 | QDISKY |
| | (in Examples 6 and 7) | |
| 8 | Anti-CD19 CDR-L2 | HTS |
| | (in Examples 6 and 7) | |
| 9 | Anti-CD19 CDR-L3 | QQGNTLPYT |
| | (in Examples 6 and 7) | |
| 10 | Anti-CD19 VL | DIQMTQTTSSLSASLGDRVTISCRASQDISK |
| | (in Examples 6 and 7) | YLNWYQQKPDGTVKLLIYHTSRLHSGVPS |
| | | RFSGSGSGTDYSLTISNLEQEDIATYFCQQ |
| | | GNTLPYTFGGGTKLEIT |
| 11 | Anti-CD19 CDR-H1 | SGVSLPDY |
| | (axicabtagene ciloleucel) | |
| 12 | Anti-CD19 CDR-H2 | LEWLGVIWGSETTYY |
| | (axicabtagene ciloleucel) | |
| 13 | Anti-CD19 CDR-H3 | HYYYGGSYAMD |
| | (axicabtagene ciloleucel) | |
| 14 | Anti-CD19 VH | KLQESGPGLVAPSQSLSVTCTVSGVSLPDY |
| | (axicabtagene ciloleucel) | GVSWIRQPPRKGLEWLGVIWGSETTYYNS |
| | | ALKSRLTIIKDNSKSQVFLKMNSLQTDDTA |
| | | IYYCAKHYYYGGSYAMDYWGQGTSVTV |
| | | S |
| 15 | Anti-CD19 CDR-L1 | QDISKY |
| | (axicabtagene ciloleucel) | |
| 16 | Anti-CD19 CDR-L2 | HTS |
| | (axicabtagene ciloleucel) | |
| 17 | Anti-CD19 CDR-L3 | QQGNTLPYT |
| | (axicabtagene ciloleucel) | |
| 18 | Anti-CD19 VL | CELPHPAFLLIPDIQMTQTTSSLSASLGDRV |
| | (axicabtagene ciloleucel) | TISCRASQDISKYLNWYQQKPDGTVKLLIY |
| | | HTSRLHSGVPSRFSGSGSGTDYSLTISNLE |
| | | QEDIATYFCQQGNTLPYTFGGGTKLEITG |
| 19 | Anti-CD19 CDR-H1 | GVSLPDYG |
| | (tisagenlecleucel) | |
| 20 | Anti-CD19 CDR-H2 | IWGSETT |
| | (tisagenlecleucel) | |
| 21 | Anti-CD19 CDR-H3 | AKHYYYGGSYAMDY |
| | (tisagenlecleucel) | |
| 22 | Anti-CD19 VH | GVSLPDYGVSWIRQPPRKGLEWLGVIWGS |
| | (tisagenlecleucel) | ETTYYNSALKSRLTIIKDNSKSQVFLKMNS |
| | | LQTDDTAIYYCAKHYYYGGSYAMDYWG |
| | | QGTSVTVS |
| 23 | Anti-CD19 CDR-L1 | QDISKY |
| | (tisagenlecleucel) | |
| 24 | Anti-CD19 CDR-L2 | HTS |
| | (tisagenlecleucel) | |
| 25 | Anti-CD19 CDR-L3 | QQGNTLPYT |
| | (tisagenlecleucel) | |
| 26 | Anti-CD19 VL | PDIQMTQTTSSLSASLGDRVTISCRASQDIS |
| | (tisagenlecleucel) | KYLNWYQQKPDGTVKLLIYHTSRLHSGVP |
| | | SRFSGSGSGTDYSLTISNLEQEDIATYFCQQ |
| | | GNTLPYTFGGGTKLEITGGGGSGGGGSGG |
| | | GGSEVKLQESGPGLVAPSQSLSVTCTVS |
| 27 | Anti-CD22 CDR-H1 | GDSVSSNSAA |
| | (in Examples 6 and 7) | |
| 28 | Anti-CD22 CDR-H2 | TYYRSKWYN |
| | (in Examples 6 and 7) | |
| 29 | Anti-CD22 CDR-H3 | AREVTGDLEDAFDI |
| | (in Examples 6 and 7) | |
| 30 | Anti-CD22 VH | QVQLQQSGPGLVKPSQTLSLTCAISGDSVS |
| | (in Examples 6 and 7) | SNSAAWNWIRQSPSRGLEWLGRTYYRSK |
| | | WYNDYAVSVKSRITINPDTSKNQFSLQLN |
| | | SVTPEDTAVYYCAREVTGDLEDAFDIWGQ |
| | | GTMVTVSS |
| 31 | Anti-CD22 CDR-L1 | QTIWSY |
| | (in Examples 6 and 7) | |
| 32 | Anti-CD22 CDR-L2 | AAS |
| | (in Examples 6 and 7) | |
| 33 | Anti-CD22 CDR-L3 | QQSYSIPQT |
| | (in Examples 6 and 7) | |
| 34 | Anti-CD22 VL | DIQMTQSPSSLSASVGDRVTITCRASQTIW |
| | (in Examples 6 and 7) | SYLNWYQQRPGKAPNLLIYAASSLQSGVP |
| | | SRFSGRGSGTDFTLTISSLQAEDFATYYCQ |
| | | QSYSIPQTFGQGTKLEIK |
| 35 | CD8α Chain | TTTPAPRPPTPAPTIASQPLSLRPEACRPAA |
| | (in Examples 6 and 7) | GGAVHTRGLDFACDIYIWAPLAGTCGVLL |
| | | LSLVITLYC |
| 36 | CD8α Chain | TTTPAPRPPTPAPTIASQPLSLRPEACRPAA |
| | (tisagenlecleucel) | GGAVHTRGLDFACDIYIWAPLAGTCGVLL |
| | | LSLVITLYC |
| 37 | 4-1BB Domain | KRGRKKLLYIFKQPFMRPVQTTQEEDGCS |
| | (in Examples 6 and 7) | CRFPEEEEGGCEL |
| 38 | 4-1BB Domain | KKLLYIFKQPFMRPVQTTQEEDGCSCRFPE |
| | (tisagenlecleucel) | EEEGGCEL |
| 39 | CD28 Domain | IEVMYPPPYLDNEKSNGTIIHVKGKHLCPS |
| | (axicabtagene ciloleucel) | PLFPGPSKPFWVLVVVGGVLACYSLLVTV |
| | | AFIIFWVRSKRSRLLHSDYMNMTPRRPGPT |
| | | RKHYQPYAPPRDFAAYRS |
| 40 | CD3ζ Domain | RVKFSRSADAPAYKQGQNQLYNELNLGR |
| | (in Examples 6 and 7) | REEYDVLDKRRGRDPEMGGKPRRKNPQE |
| | | GLYNELQKDKMAEAYSEIGMKGERRRGK |
| | | GHDGLYQGLSTATKDTYDALHMQALPPR |
| 41 | CD3ζ Domain | RVKFSRSADAPAYQQGQNQLYNELNLGR |
| | (axicabtagene ciloleucel) | REEYDVLDKRRGRDPEMGGKPRRKNPQE |
| | | GLYNELQKDKMAEAYSEIGMKGERRRGK |
| | | GHDGLYQGLSTATKDTYDALHMQALPPR |
| 42 | CD3ζ Domain | RVKFSRSADAPAYKQGQNQLYNELNLGR |
| | (tisagenlecleucel) | REEYDVLDKRRGRDPEMGGKPRRKNPQE |
| | | GLYNELQKDKMAEAYSEIGMKGERRRGK |
| | | GHDGLYQGLSTATKDTYDALHMQALPPR |
| 43 | CAR Amino Acid | SATMLLLVTSLLLCELPHPAFLLIPDIQMT |
| | (in Examples 6 and 7) | QTTSSLSASLGDRVTISCRASQDISKYLNW |
| | | YQQKPDGTVKLLIYHTSRLHSGVPSRFSGS |
| | | GSGTDYSLTISNLEQEDIATYFCQQGNTLP |
| | | YTFGGGTKLEITGGGGSQVQLQQSGPGLV |
| | | KPSQTLSLTCAISGDSVSSNSAAWNWIRQS |
| | | PSRGLEWLGRTYYRSKWYNDYAVSVKSR |
| | | ITINPDTSKNQFSLQLNSVTPEDTAVYYCA |
| | | REVTGDLEDAFDIWGQGTMVTVSSGSTSG |
| | | SGKPGSGEGSTKGDIQMTQSPSSLSASVGD |
| | | RVTITCRASQTIWSYLNWYQQRPGKAPNL |
| | | LIYAASSLQSGVPSRFSGRGSGTDFTLTISS |
| | | LQAEDFATYYCQQSYSIPQTFGQGTKLEIK |
| | | GGGGSEVKLQESGPGLVAPSQSLSVTCTV |
| | | SGVSLPDYGVSWIRQPPRKGLEWLGVIWG |
| | | SETTYYNSALKSRLTIIKDNSKSQVFLKMN |
| | | SLQTDDTAIYYCAKHYYYGGSYAMDYW |
| | | GQGTSVTVSSSGTTTPAPRPPTPAPTIASQP |
| | | LSLRPEACRPAAGGAVHTRGLDFACDIYI |
| | | WAPLAGTCGVLLLSLVITLYCKRGRKKLL |
| | | YIFKQPFMRPVQTTQEEDGCSCRFPEEEEG |
| | | GCELRVKFSRSADAPAYKQGQNQLYNEL |
| | | NLGRREEYDVLDKRRGRDPEMGGKPRRK |
| | | NPQEGLYNELQKDKMAEAYSEIGMKGER |
| | | RRGKGHDGLYQGLSTATKDTYDALHMQ |
| | | ALPPR |
| 44 | CAR Nucleotide | CTAGCGCCACCATGCTGCTGCTCGTGAC |
| | (in Examples 6 and 7) | AAGCCTGCTGCTGTGCGAGCTGCCCCAC |
| | | CCTGCCTTTCTGCTGATCCCCGACATCCA |
| | | GATGACCCAGACCACCAGCAGCCTGAGC |
| | | GCCAGCCTGGGCGATAGAGTGACCATCA |
| | | GCTGCAGAGCCAGCCAGGACATCAGCA |
| | | AGTACCTGAACTGGTATCAGCAGAAACC |
| | | CGACGGCACCGTGAAGCTGCTGATCTAC |
| | | CACACCAGCAGACTGCACAGCGGCGTGC |
| | | CCAGCAGATTTTCTGGCAGCGGCTCCGG |
| | | CACCGACTACAGCCTGACCATCTCCAAC |
| | | CTGGAACAGGAAGATATCGCTACCTACT |
| | | TCTGTCAGCAAGGCAACACCCTGCCCTA |
| | | CACCTTCGGCGGAGGCACCAAGCTGGAA |
| | | ATCACAGGCGGCGGAGGATCCCAGGTG |
| | | CAGCTGCAGCAGTCTGGACCCGGCCTCG |
| | | TGAAGCCTAGCCAGACCCTGTCTCTGAC |
| | | CTGCGCCATCAGCGGCGATAGCGTGTCC |
| | | AGCAATAGCGCCGCCTGGAACTGGATCC |
| | | GGCAGAGCCCTTCTAGAGGCCTGGAATG |
| | | GCTGGGCCGGACCTACTACCGGTCCAAG |
| | | TGGTACAACGACTACGCCGTGTCCGTGA |
| | | AGTCCCGGATCACCATCAACCCCGACAC |
| | | CAGCAAGAACCAGTTCTCCCTGCAGCTG |
| | | AACAGCGTGACCCCCGAGGATACCGCCG |
| | | TGTACTACTGCGCCAGAGAAGTGACCGG |
| | | CGACCTGGAAGATGCCTTCGACATCTGG |
| | | GGCCAGGGCACAATGGTCACCGTGTCTA |
| | | GCGGCAGCACAAGCGGCTCTGGCAAGC |
| | | CTGGATCTGGCGAGGGCTCTACCAAGGG |
| | | CGATATTCAGATGACACAGAGCCCCTCC |
| | | AGCCTGTCCGCCTCTGTGGGAGACAGAG |
| | | TGACAATCACCTGTCGGGCCTCCCAGAC |
| | | CATCTGGTCCTATCTGAATTGGTATCAG |
| | | CAGCGGCCTGGCAAGGCCCCCAACCTGC |
| | | TGATCTATGCCGCCAGCTCTCTGCAGTC |
| | | CGGCGTGCCATCTAGATTCAGCGGCAGA |
| | | GGCAGCGGCACCGATTTCACCCTGACAA |
| | | TTAGCAGTCTGCAGGCCGAGGACTTCGC |
| | | CACCTACTATTGCCAGCAGAGCTACAGC |
| | | ATCCCCCAGACCTTCGGCCAGGGAACAA |
| | | AACTGGAAATCAAAGGGGGAGGCGGCA |
| | | GCGAAGTGAAACTGCAGGAATCTGGCCC |
| | | TGGCCTGGTGGCCCCAAGCCAGTCTCTG |
| | | AGCGTGACCTGTACCGTGTCTGGCGTGT |
| | | CCCTGCCCGATTACGGCGTGTCCTGGAT |
| | | CAGACAGCCCCCCAGAAAGGGACTGGA |
| | | ATGGCTGGGAGTGATCTGGGGCAGCGA |
| | | GACAACCTACTACAACAGCGCCCTGAAG |
| | | TCCAGGCTGACCATCATCAAGGACAACT |
| | | CCAAGAGCCAGGTGTTCCTGAAGATGAA |
| | | TTCCCTGCAGACCGACGACACCGCCATC |
| | | TATTACTGTGCCAAGCACTACTACTACG |
| | | GCGGCAGCTACGCCATGGACTACTGGGG |
| | | ACAGGGAACCTCCGTGACCGTGTCCTCT |
| | | TCCGGAACCACGACGCCAGCGCCGCGAC |
| | | CACCAACACCGGCGCCCACCATCGCGTC |
| | | GCAGCCCCTGTCCCTGCGCCCAGAGGCG |
| | | TGCCGGCCAGCGGCGGGGGGCGCAGTG |
| | | CACACGAGGGGGCTGGACTTCGCCTGTG |
| | | ATATCTACATCTGGGCGCCCTTGGCCGG |
| | | GACTTGTGGGGTCCTTCTCCTGTCACTGG |
| | | TTATCACCCTTTACTGCAAACGGGGCAG |
| | | AAAGAAACTCCTGTATATATTCAAACAA |
| | | CCATTTATGAGACCAGTACAAACTACTC |
| | | AAGAGGAAGATGGCTGTAGCTGCCGATT |
| | | TCCAGAAGAAGAAGAAGGAGGATGTGA |
| | | ACTGAGAGTGAAGTTCAGCAGGAGCGC |
| | | AGACGCCCCCGCGTACAAGCAGGGCCA |
| | | GAACCAGCTCTATAACGAGCTCAATCTA |
| | | GGACGAAGAGAGGAGTACGATGTTTTG |
| | | GACAAGAGACGTGGCCGGGACCCTGAG |
| | | ATGGGGGGAAAGCCGAGAAGGAAGAAC |
| | | CCTCAGGAAGGCCTGTACAATGAACTGC |
| | | AGAAAGATAAGATGGCGGAGGCCTACA |
| | | GTGAGATTGGGATGAAAGGCGAGCGCC |
| | | GGAGGGGCAAGGGGCACGATGGCCTTT |
| | | ACCAGGGTCTCAGTACAGCCACCAAGGA |
| | | CACCTACGACGCCCTTCACATGCAGGCC |
| | | CTGCCCCCTCGCTAAGTC |
| 45 | CAR Amino Acid | MLLLVTSLLLCELPHPAFLLIPDIQMTQTTS |
| | (axicabtagene ciloleucel) | SLSASLGDRVTISCRASQDISKYLNWYQQ |
| | | KPDGTVKLLIYHTSRLHSGVPSRFSGSGSG |
| | | TDYSLTISNLEQEDIATYFCQQGNTLPYTF |
| | | GGGTKLEITGSTSGSGKPGSGEGSTKGEVK |
| | | LQESGPGLVAPSQSLSVTCTVSGVSLPDYG |
| | | VSWIRQPPRKGLEWLGVIWGSETTYYNSA |
| | | LKSRLTIIKDNSKSQVFLKMNSLQTDDTAI |
| | | YYCAKHYYYGGSYAMDYWGQGTSVTVS |
| | | SAAAIEVMYPPPYLDNEKSNGTIIHVKGKH |
| | | LCPSPLFPGPSKPFWVLVVVGGVLACYSL |
| | | LVTVAFIIFWVRSKRSRLLHSDYMNMTPR |
| | | RPGPTRKHYQPYAPPRDFAAYRSRVKFSR |
| | | SADAPAYQQGQNQLYNELNLGRREEYDV |
| | | LDKRRGRDPEMGGKPRRKNPQEGLYNEL |
| | | QKDKMAEAYSEIGMKGERRRGKGHDGLY |
| | | QGLSTATKDTYDALHMQALPPR |
| 46 | CAR Nucleotide | ATGCTTCTCCTGGTGACAAGCCTTCTGCT |
| | (axicabtagene ciloleucel) | CTGTGAGTTACCACACCCAGCATTCCTC |
| | | CTGATCCCAGACATCCAGATGACACAGA |
| | | CTACATCCTCCCTGTCTGCCTCTCTGGGA |
| | | GACAGAGTCACCATCAGTTGCAGGGCAA |
| | | GTCAGGACATTAGTAAATATTTAAATTG |
| | | GTATCAGCAGAAACCAGATGGAACTGTT |
| | | AAACTCCTGATCTACCATACATCAAGAT |
| | | TACACTCAGGAGTCCCATCAAGGTTCAG |
| | | TGGCAGTGGGTCTGGAACAGATTATTCT |
| | | CTCACCATTAGCAACCTGGAGCAAGAAG |
| | | ATATTGCCACTTACTTTTGCCAACAGGG |
| | | TAATACGCTTCCGTACACGTTCGGAGGG |
| | | GGGACTAAGTTGGAAATAACAGGCTCCA |
| | | CCTCTGGATCCGGCAAGCCCGGATCTGG |
| | | CGAGGGATCCACCAAGGGCGAGGTGAA |
| | | ACTGCAGGAGTCAGGACCTGGCCTGGTG |
| | | GCGCCCTCACAGAGCCTGTCCGTCACAT |
| | | GCACTGTCTCAGGGGTCTCATTACCCGA |
| | | CTATGGTGTAAGCTGGATTCGCCAGCCT |
| | | CCACGAAAGGGTCTGGAGTGGCTGGGA |
| | | GTAATATGGGGTAGTGAAACCACATACT |
| | | ATAATTCAGCTCTCAAATCCAGACTGAC |
| | | CATCATCAAGGACAACTCCAAGAGCCAA |
| | | GTTTTCTTAAAAATGAACAGTCTGCAAA |
| | | CTGATGACACAGCCATTTACTACTGTGC |
| | | CAAACATTATTACTACGGTGGTAGCTAT |
| | | GCTATGGACTACTGGGGTCAAGGAACCT |
| | | CAGTCACCGTCTCCTCAGCGGCCGCAAT |
| | | TGAAGTTATGTATCCTCCTCCTTACCTAG |
| | | ACAATGAGAAGAGCAATGGAACCATTA |
| | | TCCATGTGAAAGGGAAACACCTTTGTCC |
| | | AAGTCCCCTATTTCCCGGACCTTCTAAG |
| | | CCCTTTTGGGTGCTGGTGGTGGTTGGGG |
| | | GAGTCCTGGCTTGCTATAGCTTGCTAGT |
| | | AACAGTGGCCTTTATTATTTTCTGGGTGA |
| | | GGAGTAAGAGGAGCAGGCTCCTGCACA |
| | | GTGACTACATGAACATGACTCCCCGCCG |
| | | CCCCGGGCCCACCCGCAAGCATTACCAG |
| | | CCCTATGCCCCACCACGCGACTTCGCAG |
| | | CCTATCGCTCCAGAGTGAAGTTCAGCAG |
| | | GAGCGCAGACGCCCCCGCGTACCAGCA |
| | | GGGCCAGAACCAGCTCTATAACGAGCTC |
| | | AATCTAGGACGAAGAGAGGAGTACGAT |
| | | GTTTTGGACAAGAGACGTGGCCGGGACC |
| | | CTGAGATGGGGGGAAAGCCGAGAAGGA |
| | | AGAACCCTCAGGAAGGCCTGTACAATGA |
| | | ACTGCAGAAAGATAAGATGGCGGAGGC |
| | | CTACAGTGAGATTGGGATGAAAGGCGA |
| | | GCGCCGGAGGGGCAAGGGGCACGATGG |
| | | CCTTTACCAGGGTCTCAGTACAGCCACC |
| | | AAGGACACCTACGACGCCCTTCACATGC |
| | | AGGCCCTGCCCCCTCGCTAA |
| 47 | CAR Amino Acid | MALPVTALLLPLALLLHAARPDIQMTQTT |
| | (tisagenlecleucel) | SSLSASLGDRVTISCRASQDISKYLNWYQQ |
| | | KPDGTVKLLIYHTSRLHSGVPSRFSGSGSG |
| | | TDYSLTISNLEQEDIATYFCQQGNTLPYTF |
| | | GGGTKLEITGGGGSGGGGSGGGGSEVKLQ |
| | | ESGPGLVAPSQSLSVTCTVSGVSLPDYGVS |
| | | WIRQPPRKGLEWLGVIWGSETTYYNSALK |
| | | SRLTIIKDNSKSQVFLKMNSLQTDDTAIYY |
| | | CAKHYYYGGSYAMDYWGQGTSVTVSST |
| | | TTPAPRPPTPAPTIASQPLSLRPEACRPAAG |
| | | GAVHTRGLDFACDIYIWAPLAGTCGVLLL |
| | | SLVITLYCKRGRKKLLYIFKQPFMRPVQTT |
| | | QEEDGCSCRFPEEEEGGCELRVKFSRSAD |
| | | APAYKQGQNQLYNELNLGRREEYDVLDK |
| | | RRGRDPEMGGKPRRKNPQEGLYNELQKD |
| | | KMAEAYSEIGMKGERRRGKGHDGLYQGL |
| | | STATKDTYDALHMQALPPR |
| 48 | CAR Nucleotide | ATGGCCTTACCAGTGACCGCCTTGCTCC |
| | (tisagenlecleucel) | TGCCGCTGGCCTTGCTGCTCCACGCCGC |
| | | CAGGCCGGACATCCAGATGACACAGACT |
| | | ACATCCTCCCTGTCTGCCTCTCTGGGAG |
| | | ACAGAGTCACCATCAGTTGCAGGGCAAG |
| | | TCAGGACATTAGTAAATATTTAAATTGG |
| | | TATCAGCAGAAACCAGATGGAACTGTTA |
| | | AACTCCTGATCTACCATACATCAAGATT |
| | | ACACTCAGGAGTCCCATCAAGGTTCAGT |
| | | GGCAGTGGGTCTGGAACAGATTATTCTC |
| | | TCACCATTAGCAACCTGGAGCAAGAAGA |
| | | TATTGCCACTTACTTTTGCCAACAGGGT |
| | | AATACGCTTCCGTACACGTTCGGAGGGG |
| | | GGACCAAGCTGGAGATCACAGGTGGCG |
| | | GTGGCTCGGGCGGTGGTGGGTCGGGTGG |
| | | CGGCGGATCTGAGGTGAAACTGCAGGA |
| | | GTCAGGACCTGGCCTGGTGGCGCCCTCA |
| | | CAGAGCCTGTCCGTCACATGCACTGTCT |
| | | CAGGGGTCTCATTACCCGACTATGGTGT |
| | | AAGCTGGATTCGCCAGCCTCCACGAAAG |
| | | GGTCTGGAGTGGCTGGGAGTAATATGGG |
| | | GTAGTGAAACCACATACTATAATTCAGC |
| | | TCTCAAATCCAGACTGACCATCATCAAG |
| | | GACAACTCCAAGAGCCAAGTTTTCTTAA |
| | | AAATGAACAGTCTGCAAACTGATGACAC |
| | | AGCCATTTACTACTGTGCCAAACATTAT |
| | | TACTACGGTGGTAGCTATGCTATGGACT |
| | | ACTGGGGCCAAGGAACCTCAGTCACCGT |
| | | CTCCTCAACCACGACGCCAGCGCCGCGA |
| | | CCACCAACACCGGCGCCCACCATCGCGT |
| | | CGCAGCCCCTGTCCCTGCGCCCAGAGGC |
| | | GTGCCGGCCAGCGGCGGGGGGCGCAGT |
| | | GCACACGAGGGGGCTGGACTTCGCCTGT |
| | | GATATCTACATCTGGGCGCCCTTGGCCG |
| | | GGACTTGTGGGGTCCTTCTCCTGTCACTG |
| | | GTTATCACCCTTTACTGCAAACGGGGCA |
| | | GAAAGAAACTCCTGTATATATTCAAACA |
| | | ACCATTTATGAGACCAGTACAAACTACT |
| | | CAAGAGGAAGATGGCTGTAGCTGCCGAT |
| | | TTCCAGAAGAAGAAGAAGGAGGATGTG |
| | | AACTGAGAGTGAAGTTCAGCAGGAGCG |
| | | CAGACGCCCCCGCGTACAAGCAGGGCC |
| | | AGAACCAGCTCTATAACGAGCTCAATCT |
| | | AGGACGAAGAGAGGAGTACGATGTTTT |
| | | GGACAAGAGACGTGGCCGGGACCCTGA |
| | | GATGGGGGGAAAGCCGAGAAGGAAGAA |
| | | CCCTCAGGAAGGCCTGTACAATGAACTG |
| | | CAGAAAGATAAGATGGCGGAGGCCTAC |
| | | AGTGAGATTGGGATGAAAGGCGAGCGC |
| | | CGGAGGGGCAAGGGGCACGATGGCCTT |
| | | TACCAGGGTCTCAGTACAGCCACCAAGG |
| | | ACACCTACGACGCCCTTCACATGCAGGC |
| | | CCTGCCCCCTCGC |
| 49 | Anti-BCMA CDR-H1 | GYTFTDYS |
| | (idecabtagene vicleucel) | |
| 50 | Anti-BCMA CDR-H2 | INTETREP |
| | (idecabtagene vicleucel) | |
| 51 | Anti-BCMA CDR-H3 | ALDYSYAMDY |
| | (idecabtagene vicleucel) | |
| 52 | Anti-BCMA VH | QLVQSGPELKKPGETVKISCKASGYTFTD |
| | (idecabtagene vicleucel) | YSINWVKRAPGKGLKWMGWINTETREPA |
| | | YAYDFRGRFAFSLETSASTAYLQINNLKYE |
| | | DTATYFCALDYSYAMDYWGQGTSVTVSS |
| 53 | Anti-BCMA CDR-L1 | ESVTILGSHL |
| | (idecabtagene vicleucel) | |
| 54 | Anti-BCMA CDR-L2 | LAS |
| | (idecabtagene vicleucel) | |
| 55 | Anti-BCMA CDR-L3 | LQSRTIPRT |
| | (idecabtagene vicleucel) | |
| 56 | Anti-BCMA VL | IVLTQSPPSLAMSLGKRATISCRASESVTIL |
| | (idecabtagene vicleucel) | GSHLIHWYQQKPGQPPTLLIQLASNVQTG |
| | | VPARFSGSGSRTDFTLTIDPVEEDDVAVYY |
| | | CLQSRTIPRTFGGGTKLEI |
| 57 | CAR Amino Acid | MALPVTALLLPLALLLHAARPDIVLTQSPP |
| | (idecabtagene vicleucel) | SLAMSLGKRATISCRASESVTILGSHLIHW |
| | | YQQKPGQPPTLLIQLASNVQTGVPARFSGS |
| | | GSRTDFTLTIDPVEEDDVAVYYCLQSRTIP |
| | | RTFGGGTKLEIKGSTSGSGKPGSGEGSTKG |
| | | QIQLVQSGPELKKPGETVKISCKASGYTFT |
| | | DYSINWVKRAPGKGLKWMGWINTETREP |
| | | AYAYDFRGRFAFSLETSASTAYLQINNLK |
| | | YEDTATYFCALDYSYAMDYWGQGTSVTV |
| | | SSAAATTTPAPRPPTPAPTIASQPLSLRPEA |
| | | CRPAAGGAVHTRGLDFACDIYIWAPLAGT |
| | | CGVLLLSLVITLYCKRGRKKLLYIFKQPFM |
| | | RPVQTTQEEDGCSCRFPEEEEGGCELRVKF |
| | | SRSADAPAYQQGQNQLYNELNLGRREEY |
| | | DVLDKRRGRDPEMGGKPRRKNPQEGLYN |
| | | ELQKDKMAEAYSEIGMKGERRRGKGHDG |
| | | LYQGLSTATKDTYDALHMQALPPR |
| 58 | CAR Nucleotide | ATGGCACTCCCCGTCACCGCCCTTCTCTT |
| | (idecabtagene vicleucel) | GCCCCTCGCCCTGCTGCTGCATGCTGCC |
| | | AGGCCCGACATTGTGCTCACTCAGTCAC |
| | | CTCCCAGCCTGGCCATGAGCCTGGGAAA |
| | | AAGGGCCACCATCTCCTGTAGAGCCAGT |
| | | GAGTCCGTCACAATCTTGGGGAGCCATC |
| | | TTATTCACTGGTATCAGCAGAAGCCCGG |
| | | GCAGCCTCCAACCCTTCTTATTCAGCTCG |
| | | CGTCAAACGTCCAGACGGGTGTACCTGC |
| | | CAGATTTTCTGGTAGCGGGTCCCGCACT |
| | | GATTTTACACTGACCATAGATCCAGTGG |
| | | AAGAAGACGATGTGGCCGTGTATTATTG |
| | | TCTGCAGAGCAGAACGATTCCTCGCACA |
| | | TTTGGTGGGGGTACTAAGCTGGAGATTA |
| | | AGGGAAGCACGTCCGGCTCAGGGAAGC |
| | | CGGGCTCCGGCGAGGGAAGCACGAAGG |
| | | GGCAAATTCAGCTGGTCCAGAGCGGACC |
| | | TGAGCTGAAAAAACCCGGCGAGACTGTT |
| | | AAGATCAGTTGTAAAGCATCTGGCTATA |
| | | CCTTCACCGACTACAGCATAAATTGGGT |
| | | GAAACGGGCCCCTGGAAAGGGCCTCAA |
| | | ATGGATGGGTTGGATCAATACCGAAACT |
| | | AGGGAGCCTGCTTATGCATATGACTTCC |
| | | GCGGGAGATTCGCCTTTTCACTCGAGAC |
| | | ATCTGCCTCTACTGCTTACCTCCAAATAA |
| | | ACAACCTCAAGTATGAAGATACAGCCAC |
| | | TTACTTTTGCGCCCTCGACTATAGTTACG |
| | | CCATGGACTACTGGGGACAGGGAACCTC |
| | | CGTTACCGTCAGTTCCGCGGCCGCAACC |
| | | ACAACACCTGCTCCAAGGCCCCCCACAC |
| | | CCGCTCCAACTATAGCCAGCCAACCATT |
| | | GAGCCTCAGACCTGAAGCTTGCAGGCCC |
| | | GCAGCAGGAGGCGCCGTCCATACGCGA |
| | | GGCCTGGACTTCGCGTGTGATATTTATA |
| | | TTTGGGCCCCTTTGGCCGGAACATGTGG |
| | | GGTGTTGCTTCTCTCCCTTGTGATCACTC |
| | | TGTATTGTAAGCGCGGGAGAAAGAAGCT |
| | | CCTGTACATCTTCAAGCAGCCTTTTATGC |
| | | GACCTGTGCAAACCACTCAGGAAGAAG |
| | | ATGGGTGTTCATGCCGCTTCCCCGAGGA |
| | | GGAAGAAGGAGGGTGTGAACTGAGGGT |
| | | GAAATTTTCTAGAAGCGCCGATGCTCCC |
| | | GCATATCAGCAGGGTCAGAATCAGCTCT |
| | | ACAATGAATTGAATCTCGGCAGGCGAGA |
| | | AGAGTACGATGTTCTGGACAAGAGACG |
| | | GGGCAGGGATCCCGAGATGGGGGGAAA |
| | | GCCCCGGAGAAAAAATCCTCAGGAGGG |
| | | GTTGTACAATGAGCTGCAGAAGGACAA |
| | | GATGGCTGAAGCCTATAGCGAGATCGGA |
| | | ATGAAAGGCGAAAGACGCAGAGGCAAG |
| | | GGGCATGACGGTCTGTACCAGGGTCTCT |
| | | CTACAGCCACCAAGGACACTTATGATGC |
| | | GTTGCATATGCAAGCCTTGCCACCCCGC |
| | | TAATGA |
| 59 | Anti-BCMA CDR-H1 | EHTFSSHV |
| | (ciltacabtagene autoleucel) | |
| 60 | Anti-BCMA CDR-H2 | IGWRDIST |
| | (ciltacabtagene autoleucel) | |
| 61 | Anti-BCMA CDR-H3 | AARRIDAADFDS |
| | (ciltacabtagene autoleucel) | |
| 62 | Anti-BCMA VHH | QVKLEESGGGLVQAGRSLRLSCAASEHTF |
| | (ciltacabtagene autoleucel) | SSHVMGWFRQAPGKERESVAVIGWRDIST |
| | | SYADSVKGRFTISRDNAKKTLYLQMNSLK |
| | | PEDTAVYYCAARRIDAADFDSWGQGTQV |
| | | TVSS |
| 63 | Anti-BCMA CDR-H1 | GRTFT |
| | (ciltacabtagene autoleucel) | |
| 64 | Anti-BCMA CDR-H2 | ISLSPTLA |
| | (ciltacabtagene autoleucel) | |
| 65 | Anti-BCMA CDR-H3 | AADRKSVMSIRPDY |
| | (ciltacabtagene autoleucel) | |
| 66 | Anti-BCMA V_{H}H | EVQLVESGGGLVQAGGSLRLSCAASGRTF |
| | (ciltacabtagene autoleucel) | TMGWFRQAPGKEREFVAAISLSPTLAYYA |
| | | ESVKGRFTISRDNAKNTVVLQMNSLKPED |
| | | TALYYCAADRKSVMSIRPDYWGQGTQVT |
| | | VSS |
| 67 | CAR Amino Acid | MALPVTALLLPLALLLHAARPQVKLEESG |
| | (ciltacabtagene autoleucel) | GGLVQAGRSLRLSCAASEHTFSSHVMGW |
| | | FRQAPGKERESVAVIGWRDISTSYADSVK |
| | | GRFTISRDNAKKTLYLQMNSLKPEDTAVY |
| | | YCAARRIDAADFDSWGQGTQVTVSSGGG |
| | | GSEVQLVESGGGLVQAGGSLRLSCAASGR |
| | | TFTMGWFRQAPGKEREFVAAISLSPTLAY |
| | | YAESVKGRFTISRDNAKNTVVLQMNSLKP |
| | | EDTALYYCAADRKSVMSIRPDYWGQGTQ |
| | | VTVSSTTTPAPRPPTPAPTIASQPLSLRPEA |
| | | CRPAAGGAVHTRGLDFACDIYIWAPLAGT |
| | | CGVLLLSLVITLYCKRGRKKLLYIFKQPFM |
| | | RPVQTTQEEDGCSCRFPEEEEGGCELRVKF |
| | | SRSADAPAYQQGQNQLYNELNLGRREEY |
| | | DVLDKRRGRDPEMGGKPRRKNPQEGLYN |
| | | ELQKDKMAEAYSEIGMKGERRRGKGHDG |
| | | LYQGLSTATKDTYDALHMQALPPR |
| 68 | CAR Nucleotide | ATGGCTCTGCCCGTCACCGCTCTGCTGCT |
| | (ciltacabtagene autoleucel) | GCCTCTGGCTCTGCTGCTGCACGCTGCTC |
| | | GCCCTCAGGTCAAACTGGAAGAATCTGG |
| | | CGGAGGCCTGGTGCAGGCAGGACGGAG |
| | | CCTGCGCCTGAGCTGCGCAGCATCCGAG |
| | | CACACCTTCAGCTCCCACGTGATGGGCT |
| | | GGTTTCGGCAGGCCCCAGGCAAGGAGA |
| | | GAGAGAGCGTGGCCGTGATCGGCTGGA |
| | | GGGACATCTCCACATCTTACGCCGATTC |
| | | CGTGAAGGGCCGGTTCACCATCAGCCGG |
| | | GACAACGCCAAGAAGACACTGTATCTGC |
| | | AGATGAACAGCCTGAAGCCCGAGGACA |
| | | CCGCCGTGTACTATTGCGCAGCAAGGAG |
| | | AATCGACGCAGCAGACTTTGATTCCTGG |
| | | GGCCAGGGCACCCAGGTGACAGTGTCTA |
| | | GCGGAGGAGGAGGATCTGAGGTGCAGC |
| | | TGGTGGAGAGCGGAGGCGGCCTGGTGC |
| | | AGGCCGGAGGCTCTCTGAGGCTGAGCTG |
| | | TGCAGCATCCGGAAGAACCTTCACAATG |
| | | GGCTGGTTTAGGCAGGCACCAGGAAAG |
| | | GAGAGGGAGTTCGTGGCAGCAATCAGC |
| | | CTGTCCCCTACCCTGGCCTACTATGCCG |
| | | AGAGCGTGAAGGGCAGGTTTACCATCTC |
| | | CCGCGATAACGCCAAGAATACAGTGGTG |
| | | CTGCAGATGAACTCCCTGAAACCTGAGG |
| | | ACACAGCCCTGTACTATTGTGCCGCCGA |
| | | TCGGAAGAGCGTGATGAGCATTAGACCA |
| | | GACTATTGGGGGCAGGGAACACAGGTG |
| | | ACCGTGAGCAGCACCACGACGCCAGCG |
| | | CCGCGACCACCAACACCGGCGCCCACCA |
| | | TCGCGTCGCAGCCCCTGTCCCTGCGCCC |
| | | AGAGGCGTGCCGGCCAGCGGCGGGGGG |
| | | CGCAGTGCACACGAGGGGGCTGGACTTC |
| | | GCCTGTGATATCTACATCTGGGCGCCCT |
| | | TGGCCGGGACTTGTGGGGTCCTTCTCCT |
| | | GTCACTGGTTATCACCCTTTACTGCAAA |
| | | CGGGGCAGAAAGAAACTCCTGTATATAT |
| | | TCAAACAACCATTTATGAGACCAGTACA |
| | | AACTACTCAAGAGGAAGATGGCTGTAGC |
| | | TGCCGATTTCCAGAAGAAGAAGAAGGA |
| | | GGATGTGAACTGAGAGTGAAGTTCAGCA |
| | | GGAGCGCAGACGCCCCCGCGTACCAGC |
| | | AGGGCCAGAACCAGCTCTATAACGAGCT |
| | | CAATCTAGGACGAAGAGAGGAGTACGA |
| | | TGTTTTGGACAAGAGACGTGGCCGGGAC |
| | | CCTGAGATGGGGGGAAAGCCGAGAAGG |
| | | AAGAACCCTCAGGAAGGCCTGTACAATG |
| | | AACTGCAGAAAGATAAGATGGCGGAGG |
| | | CCTACAGTGAGATTGGGATGAAAGGCG |
| | | AGCGCCGGAGGGGCAAGGGGCACGATG |
| | | GCCTTTACCAGGGTCTCAGTACAGCCAC |
| | | CAAGGACACCTACGACGCCCTTCACATG |
| | | CAGGCCCTGCCCCCTCGCTAA |
| 69 | CD8α Chain | TTTPAPRPPTPAPTIASQPLSLRPEACRPAA |
| | (idecabtagene vicleucel) | GGAVHTRGLDFACDIYIWAPLAGTCGVLL |
| | | LSLVITLYCKRGRKKLLYIFKQPFMRPVQT |
| | | TQEEDGCSCRFPEEEEGGCEL |
| 70 | CD8α Chain | TTTPAPRPPTPAPTIASQPLSLRPEACRPAA |
| | (ciltacabtagene autoleucel) | GGAVHTRGLDFACDIYIWAPLAGTCGVLL |
| | | LSLVITLYC |
| 71 | CD137 Domain | KRGRKKLLYIFKQPFMRPVQTTQEEDGCS |
| | (ciltacabtagene autoleucel) | CRFPEEEEGGCEL |
| 72 | CD3ζ Domain | RVKFSRSADAPAYQQGQNQLYNELNLGR |
| | (idecabtagene vicleucel) | REEYDVLDKRRGRDPEMGGKPRRKNPQE |
| | | GLYNELQKDKMAEAYSEIGMKGERRRGK |
| | | GHDGLYQGLSTATKDTYDALHMQALPPR |
| 73 | CD3ζ Domain | RVKFSRSADAPAYQQGQNQLYNELNLGR |
| | (ciltacabtagene autoleucel) | REEYDVLDKRRGRDPEMGGKPRRKNPQE |
| | | GLYNELQKDKMAEAYSEIGMKGERRRGK |
| | | GHDGLYQGLSTATKDTYDALHMQALPPR |

What is claimed is:

1. A method of treating a cancer in a subject in need thereof, comprising administering to the subject (a) an IL-2 conjugate, and (b) a chimeric antigen receptor (CAR) therapy, wherein: the IL-2 conjugate comprises the amino acid sequence of SEQ ID NO: 1 wherein the amino acid at position P64 is replaced by the structure of Formula (I): 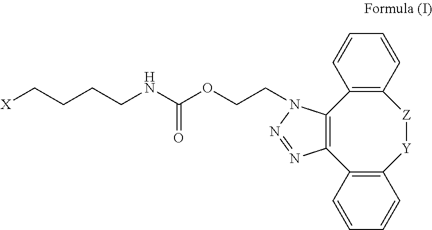
wherein:
Z is CH₂ and Y is 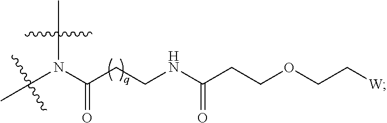
Y is CH₂ and Z is 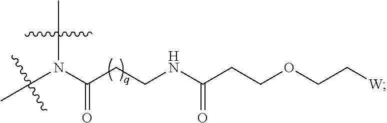
Z is CH₂ and Y is 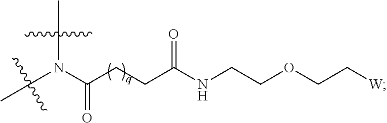
or
Y is CH₂ and Z is 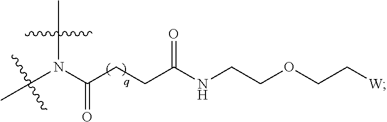
W is a PEG group having an average molecular weight of about 25 kDa-35 kDa;
q is 1, 2, or 3;
X is an L-amino acid having the structure: 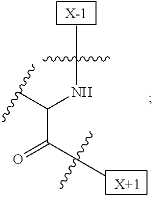
X−1 indicates the point of attachment to the preceding amino acid residue; and
X+1 indicates the point of attachment to the following amino acid residue.

2. The method of claim 1, wherein the cancer is leukemia, myeloma, or lymphoma.

3. The method of claim 2, wherein the leukemia is acute lymphoblastic leukemia or chronic lymphocytic leukemia.

4. The method of claim 2, wherein the myeloma is multiple myeloma.

5. The method of claim 2, wherein the lymphoma is non-Hodgkin lymphoma, follicular lymphoma, transformed follicular lymphoma, mantle cell lymphoma, primary mediastinal B-cell lymphoma, Burkitt's lymphoma, or diffuse large B-cell lymphoma (DLBCL).

6. The method of claim 2 or claim 5, wherein the lymphoma is Burkitt's lymphoma.

7. The method of claim 2 or claim 5, wherein the lymphoma is diffuse large B-cell lymphoma (DLBCL).

8. A method of treating diffuse large B-cell lymphoma (DLBCL) in a subject in need thereof, comprising administering to the subject an IL-2 conjugate, wherein:
the IL-2 conjugate comprises the amino acid sequence of SEQ ID NO: 1 wherein the amino acid at position P64 is replaced by the structure of Formula (I): 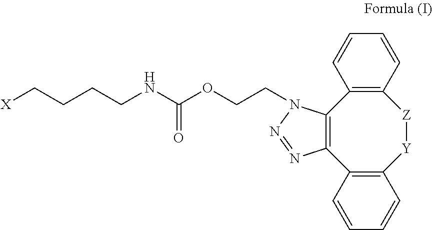
wherein:
Z is CH₂ and Y is 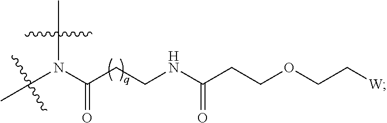
Y is CH₂ and Z is 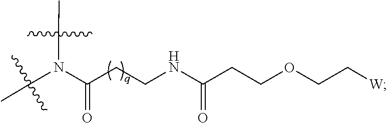
Z is CH₂ and Y is 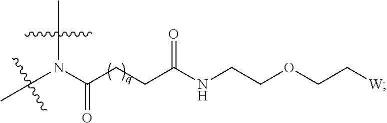
or
Y is CH₂ and Z is 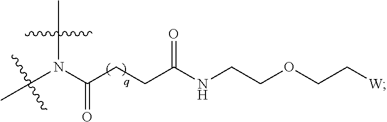
W is a PEG group having an average molecular weight of about 25 kDa-35 kDa;
q is 1, 2, or 3;
X is an L-amino acid having the structure: 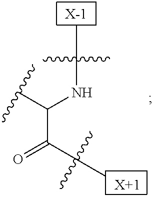
X−1 indicates the point of attachment to the preceding amino acid residue; and
X+1 indicates the point of attachment to the following amino acid residue.

9. The method of claim 7 or claim 8, wherein the DLBCL is relapsed or refractory DLBCL, or the DLBCL has relapsed after two or more prior lines of systemic therapy for DLBCL.

10. The method of any one of claims 7-9, further comprising selecting the subject to whom the IL-2 conjugate is administered at least in part on the basis of the subject having received two or more prior lines of systemic therapy for DLBCL.

11. The method of claim 9 or claim 10, wherein the two or more prior lines of systemic therapy for DLBCL comprises an anthracycline, an anti-CD20 agent, or a combination of an anthracycline and an anti-CD20 agent.

12. The method of claim 11, wherein the anti-CD20 agent comprises rituximab.

13. The method of any one of claims 9-12, wherein the two or more prior lines of systemic therapy for DLBCL comprises a CAR therapy.

14. The method of claim 13, wherein the CAR therapy is the last line of the two or more prior lines of systemic therapy for DLBCL.

15. The method of any one of claims 8-14, further comprising selecting the subject to whom the IL-2 conjugate is administered at least in part on the basis of the subject having received a CAR therapy.

16. The method of any one of claims 8-15, further comprising administering to the subject a CAR therapy.

17. The method of any one of claims 1-7 and 13-16, wherein the CAR therapy comprises T cells expressing a CAR.

18. The method of any one of claims 1-7 and 13-17, wherein the CAR therapy comprises gamma delta T cells expressing a CAR.

19. The method of any one of claims 1-7 and 13-17, wherein the CAR therapy comprises natural killer (NK) T cells expressing a CAR.

20. The method of any one of claims 1-7 and 13-16, wherein the CAR therapy comprises natural killer (NK) cells expressing a CAR.

21. The method of any one of claims 17-20, wherein the CAR comprises an anti-CD19 domain comprising an anti-CD19 heavy chain variable domain (VH) and an anti-CD19 light chain variable domain (VL).

22. The method of claim 21, wherein the anti-CD19 VH comprises a CDR-H1 comprising the amino acid sequence of SEQ ID NO: 3, a CDR-H2 comprising the amino acid sequence of SEQ ID NO: 4, and a CDR-H3 comprising the amino acid sequence of SEQ ID NO: 5.

23. The method of claim 21 or claim 22, wherein the anti-CD19 VH comprises an amino acid sequence at least 80%, 85%, 90%, 95%, 96%, 97%, 98%, or 99% identical to the amino acid sequence of SEQ ID NO: 6.

24. The method of any one of claims 21-23, wherein the anti-CD19 VH comprises the amino acid sequence of SEQ ID NO: 6.

25. The method of any one of claims 21-24, wherein the anti-CD19 VL comprises a CDR-L1 comprising the amino acid sequence of SEQ ID NO: 7, a CDR-L2 comprising the amino acid sequence of SEQ ID NO: 8, and a CDR-L3 comprising the amino acid sequence of SEQ ID NO: 9.

26. The method of any one of claims 21-25, wherein the anti-CD19 VL comprises an amino acid sequence at least 80%, 85%, 90%, 95%, 96%, 97%, 98%, or 99% identical to the amino acid sequence of SEQ ID NO: 10.

27. The method of any one of claims 21-26, wherein the anti-CD19 VL comprises the amino acid sequence of SEQ ID NO: 10.

28. The method of claim 21, wherein the anti-CD19 VH comprises a CDR-H1 comprising the amino acid sequence of SEQ ID NO: 11, a CDR-H2 comprising the amino acid sequence of SEQ ID NO: 12, and a CDR-H3 comprising the amino acid sequence of SEQ ID NO: 13.

29. The method of claim 21 or claim 28, wherein the anti-CD19 VH comprises an amino acid sequence at least 80%, 85%, 90%, 95%, 96%, 97%, 98%, or 99% identical to the amino acid sequence of SEQ ID NO: 14.

30. The method of any one of claims 21, 28, and 29, wherein the anti-CD19 VH comprises the amino acid sequence of SEQ ID NO: 14.

31. The method of any one of claims 21 and 28-30, wherein the anti-CD19 VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17.

32. The method of any one of claims 21 and 28-31, wherein the anti-CD19 VL comprises an amino acid sequence at least 80%, 85%, 90%, 95%, 96%, 97%, 98%, or 99% identical to the amino acid sequence of SEQ ID NO: 18.

33. The method of any one of claims 21 and 28-32, wherein the anti-CD19 VL comprises the amino acid sequence of SEQ ID NO: 18.

34. The method of claim 21, wherein the anti-CD19 VH comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21.

35. The method of claim 21 or claim 34, wherein the anti-CD19 VH comprises an amino acid sequence at least 80%, 85%, 90%, 95%, 96%, 97%, 98%, or 99% identical to the amino acid sequence of SEQ ID NO: 22.

36. The method of any one of claims 21, 34, and 35, wherein the anti-CD19 VH comprises the amino acid sequence of SEQ ID NO: 22.

37. The method of any one of claims 21 and 34-36, wherein the anti-CD19 VL comprises a CDR-L1 comprising the amino acid sequence of SEQ ID NO: 23, a CDR-L2 comprising the amino acid sequence of SEQ ID NO: 24, and a CDR-L3 comprising the amino acid sequence of SEQ ID NO: 25.

38. The method of any one of claims 21 and 34-37, wherein the anti-CD19 VL comprises an amino acid sequence at least 80%, 85%, 90%, 95%, 96%, 97%, 98%, or 99% identical to the amino acid sequence of SEQ ID NO: 26.

39. The method of any one of claims 21 and 34-38, wherein the anti-CD19 VL comprises the amino acid sequence of SEQ ID NO: 26.

40. The method of any one of claims 17-39, wherein the CAR comprises an anti-CD22 domain comprising an anti-CD22 heavy chain variable domain (VH) and an anti-CD22 light chain variable domain (VL).

41. The method of claim 40, wherein the anti-CD22 VH comprises a CDR-H1 comprising the amino acid sequence of SEQ ID NO: 27, a CDR-H2 comprising the amino acid sequence of SEQ ID NO: 28, and a CDR-H3 comprising the amino acid sequence of SEQ ID NO: 29.

42. The method of claim 40 or claim 41, wherein the anti-CD22 VH comprises an amino acid sequence at least 80%, 85%, 90%, 95%, 96%, 97%, 98%, or 99% identical to the amino acid sequence of SEQ ID NO: 30.

43. The method of any one of claims 40-42, wherein the anti-CD22 VH comprises the amino acid sequence of SEQ ID NO: 30.

44. The method of any one of claims 40-43, wherein the anti-CD22 VL comprises a CDR-L1 comprising the amino acid sequence of SEQ ID NO: 31, a CDR-L2 comprising the amino acid sequence of SEQ ID NO: 32, and a CDR-L3 comprising the amino acid sequence of SEQ ID NO: 33.

45. The method of any one of 40-44, wherein the anti-CD22 VL comprises an amino acid sequence at least 80%, 85%, 90%, 95%, 96%, 97%, 98%, or 99% identical to the amino acid sequence of SEQ ID NO: 34.

46. The method of any one of claims 40-45, wherein the anti-CD22 VL comprises the amino acid sequence of SEQ ID NO: 34.

47. The method of any one of claims 17-39, wherein the CAR is an anti-CD19 CAR.

48. The method of claim 47, wherein the anti-CD19 CAR comprises an amino acid sequence at least 80%, 85%, 90%, 95%, 96%, 97%, 98%, or 99% identical to the amino acid sequence of SEQ ID NO: 45.

49. The method of claim 47 or claim 48, wherein the anti-CD19 CAR comprises the amino acid sequence of SEQ ID NO: 45.

50. The method of claim 47, wherein the anti-CD19 CAR comprises an amino acid sequence at least 80%, 85%, 90%, 95%, 96%, 97%, 98%, or 99% identical to the amino acid sequence of SEQ ID NO: 47.

51. The method of claim 47 or claim 50, wherein the anti-CD19 CAR comprises the amino acid sequence of SEQ ID NO: 47.

52. The method of any one of claims 17-46, wherein the CAR is an anti-CD19/CD22 CAR.

53. The method of claim 52, wherein the anti-CD19/CD22 CAR comprises an amino acid sequence at least 80%, 85%, 90%, 95%, 96%, 97%, 98%, or 99% identical to the amino acid sequence of SEQ ID NO: 43.

54. The method of claim 52 or claim 53, wherein the anti-CD19/CD22 CAR comprises the amino acid sequence of SEQ ID NO: 43.

55. The method of any one of claims 17-20, wherein the CAR is an anti-BCMA CAR.

56. The method of claim 55, wherein the anti-BCMA CAR comprises an amino acid sequence at least 80%, 85%, 90%, 95%, 96%, 97%, 98%, or 99% identical to the amino acid sequence of SEQ ID NO: 57.

57. The method of claim 55 or claim 56, wherein the anti-BCMA CAR comprises the amino acid sequence of SEQ ID NO: 57.

58. The method of claim 55, wherein the anti-BCMA CAR comprises an amino acid sequence at least 80%, 85%, 90%, 95%, 96%, 97%, 98%, or 99% identical to the amino acid sequence of SEQ ID NO: 67.

59. The method of claim 55 or claim 56, wherein the anti-BCMA CAR comprises the amino acid sequence of SEQ ID NO: 67.

60. The method of any one of claims 17-20, wherein the CAR comprises an anti-BCMA domain comprising an anti-BCMA heavy chain variable domain (VH) and an anti-BCMA light chain variable domain (VL).

61. The method of claim 60, wherein the anti-BCMA VH comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 51.

62. The method of claim 60 or claim 61, wherein the anti-BCMA VH comprises an amino acid sequence at least 80%, 85%, 90%, 95%, 96%, 97%, 98%, or 99% identical to the amino acid sequence of SEQ ID NO: 52.

63. The method of any one of claims 60-62, wherein the anti-BCMA VH comprises the amino acid sequence of SEQ ID NO: 52.

64. The method of any one of claims 60-63, wherein the anti-BCMA VL comprises a CDR-L1 comprising the amino acid sequence of SEQ ID NO: 53, a CDR-L2 comprising the amino acid sequence of SEQ ID NO: 54, and a CDR-L3 comprising the amino acid sequence of SEQ ID NO: 55.

65. The method of any one of claims 60-64, wherein the anti-BCMA VL comprises an amino acid sequence at least 80%, 85%, 90%, 95%, 96%, 97%, 98%, or 99% identical to the amino acid sequence of SEQ ID NO: 56.

66. The method of any one of claims 60-65, wherein the anti-BCMA VL comprises the amino acid sequence of SEQ ID NO: 56.

67. The method of any one of claims 17-20, wherein the CAR comprises an anti-BCMA domain comprising a first anti-BCMA single domain antibody (V_{H}H) and/or a second anti-BCMA V_{H}H.

68. The method of claim 67, wherein the first anti-BCMA V_{H}H comprises a CDR-H1 comprising the amino acid sequence of SEQ ID NO: 59, a CDR-H2 comprising the amino acid sequence of SEQ ID NO: 60, and a CDR-H3 comprising the amino acid sequence of SEQ ID NO: 61.

69. The method of claim 67 or claim 68, wherein the first anti-BCMA V_{H}H comprises an amino acid sequence at least 80%, 85%, 90%, 95%, 96%, 97%, 98%, or 99% identical to the amino acid sequence of SEQ ID NO: 62.

70. The method of any one of claims 67-69, wherein the first anti-BCMA V_{H}H comprises the amino acid sequence of SEQ ID NO: 62.

71. The method of any one of claims 67-70, wherein the second anti-BCMA V_{H}H comprises a CDR-H1 comprising the amino acid sequence of SEQ ID NO: 63, a CDR-H2 comprising the amino acid sequence of SEQ ID NO: 64, and a CDR-H3 comprising the amino acid sequence of SEQ ID NO: 65.

72. The method of any one of claims 67-71, wherein the second anti-BCMA V_{H}H comprises an amino acid sequence at least 80%, 85%, 90%, 95%, 96%, 97%, 98%, or 99% identical to the amino acid sequence of SEQ ID NO: 66.

73. The method of any one of claims 67-72, wherein the second anti-BCMA V_{H}H comprises the amino acid sequence of SEQ ID NO: 66.

74. The method of any one of claims 13-73, wherein the CAR therapy comprises 0.2-5.0×10⁶ or 0.5-1.0×10⁶ of the cells per kg body weight of the subject.

75. The method of any one of claims 13-74, wherein the CAR therapy comprises 1.0×10⁶ or 2.0×10⁶ of the cells per kg body weight of the subject.

76. The method of any one of claims 13-75, wherein the CAR therapy comprises 0.1-2.5×10⁸ or 0.6-6.0×10⁸ of the cells.

77. The method of any one of claims 13-76, wherein the CAR therapy comprises 0.9-1.1×10⁸, 0.5-1.1×10⁸, or 3.0-4.6×10⁸ of the cells.

78. The method of any one of claims 16-77, wherein the CAR therapy is administered to the subject by intravenous administration.

79. The method of any one of claims 1-7 and 16-78, wherein the CAR therapy is administered to the subject before any dose the IL-2 conjugate is administered to the subject.

80. The method of any one of claims 1-7 and 16-78, wherein one dose of the IL-2 conjugate is administered to the subject before the CAR therapy is administered to the subject, and wherein one or more additional doses of the IL-2 conjugate is administered to the subject after the CAR therapy is administered to the subject.

81. The method of any one of claims 1-7 and 16-78, wherein one dose of the IL-2 conjugate is administered to the subject one day before the CAR therapy is administered to the subject, and wherein one or more additional doses of the IL-2 conjugate is administered to the subject about once every week after the one dose of the IL-2 conjugate is administered to the subject.

82. The method of any one of claims 1-81, comprising administering to the subject about 16 μg/kg IL-2 as the IL-2 conjugate.

83. The method of any one of claims 1-81, comprising administering to the subject about 24 μg/kg IL-2 as the IL-2 conjugate.

84. The method of any one of claims 1-81, comprising administering to the subject about 32 μg/kg IL-2 as the IL-2 conjugate.

85. The method of any one of claims 1-84, wherein in the IL-2 conjugate the PEG group has an average molecular weight of about 30 kDa.

86. The method of any one of claims 1-85, wherein in the IL-2 conjugate Z is CH₂ and Y is

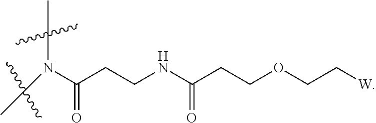

87. The method of any one of claims 1-85, wherein in the IL-2 conjugate Y is CH₂ and Z is

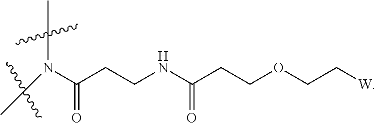

88. The method of any one of claims 1-85, wherein in the IL-2 conjugate Z is CH₂ and Y is

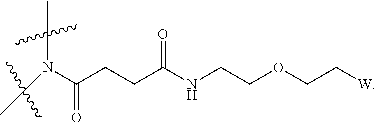

89. The method of any one of claims 1-85, wherein in the IL-2 conjugate Y is CH₂ and Z is

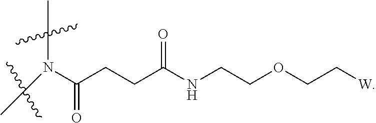

90. The method of any one of claims 1-85, wherein the structure of Formula (I) has the structure of Formula (IV) or Formula (V), or is a mixture of Formula (IV) and Formula (V): 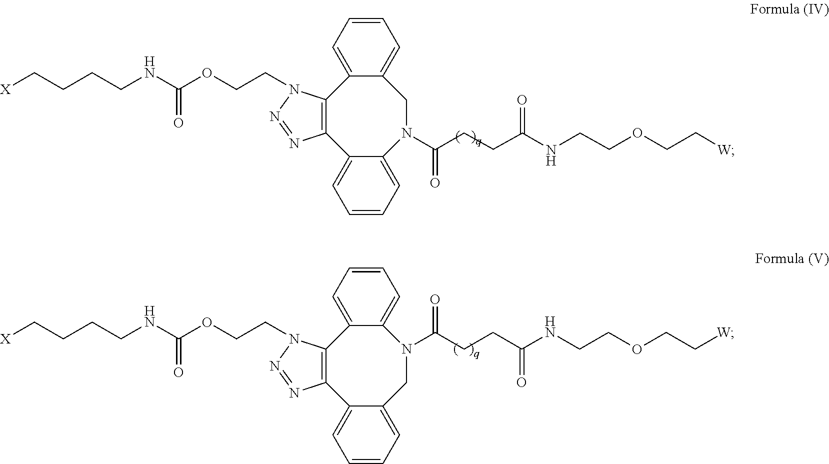
wherein:
q is 1, 2, or 3;
X is an L-amino acid having the structure: 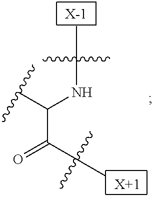
X−1 indicates the point of attachment to the preceding amino acid residue; and
X+1 indicates the point of attachment to the following amino acid residue.

91. The method of any one of claims 1-85, wherein the structure of Formula (I) has the structure of Formula (XII) or Formula (XIII), or is a mixture of Formula (XII) and Formula (XIII): 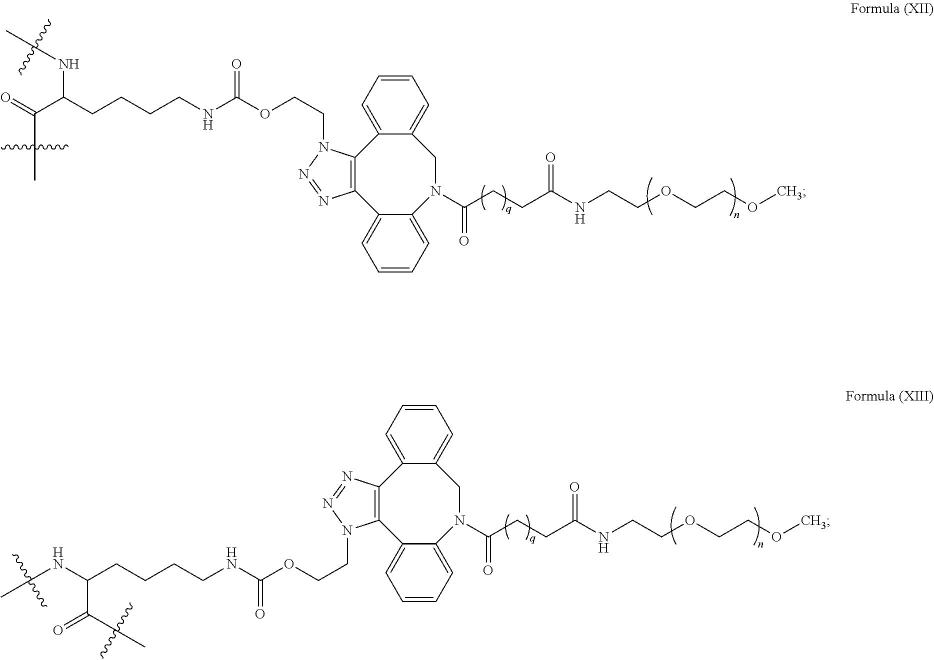
wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

92. The method of any one of claims 1-91, wherein q is 1.

93. The method of any one of claims 1-91, wherein q is 2.

94. The method of any one of claims 1-91, wherein q is 3.

95. The method of any one of claims 1-94, wherein the IL-2 conjugate is administered to the subject about once every week, about once every two weeks, about once every three weeks, or about once every 4 weeks.

96. The method of claim 95, wherein the IL-2 conjugate is administered to the subject about once every three weeks.

97. The method of any one of claims 1-96, wherein the IL-2 conjugate is a pharmaceutically acceptable salt, solvate, or hydrate.

98. The method of any one of claims 1-97, wherein the IL-2 conjugate is administered to the subject by intravenous administration.

99. The method of any one of claims 1-97, wherein the IL-2 conjugate is administered to the subject by subcutaneous administration.

100. The method of any one of claims 1-99, further comprising administering acetaminophen to the subject.

101. The method of any one of claims 1-100, further comprising administering diphenhydramine to the subject.

102. The method of claim 100 or claim 101, wherein the acetaminophen and/or diphenhydramine is administered to the subject before administering the IL-2 conjugate.

103. An IL-2 conjugate for use in the method of any one of claims 1-102.

104. Use of an IL-2 conjugate for the manufacture of a medicament for the method of any one of claims 1-102.


SEQUENCE LISTING (73 sequences)

SEQ ID NO: 1 - protein, 132 aa, synthetic construct
  REGION 1..132: note = IL-2
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
  REGION 1..132: note = IL-2 conjugate
  SITE 64: note = misc_feature - Xaa is AzK_L1_PEG30kD
PTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 3 - protein, 8 aa, synthetic construct
  REGION 1..8: note = Anti-CD19 CDR-H1
GVSLPDYG

SEQ ID NO: 4 - protein, 7 aa, synthetic construct
  REGION 1..7: note = Anti-CD19 CDR-H2
IWGSETT

SEQ ID NO: 5 - protein, 14 aa, synthetic construct
  REGION 1..14: note = Anti-CD19 CDR-H3
AKHYYYGGSYAMDY

SEQ ID NO: 6 - protein, 120 aa, synthetic construct
  REGION 1..120: note = Anti-CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - protein, 6 aa, synthetic construct
  REGION 1..6: note = Anti-CD19 CDR-L1
QDISKY

SEQ ID NO: 8 - skipped

SEQ ID NO: 9 - protein, 9 aa, synthetic construct
  REGION 1..9: note = Anti-CD19 CDR-L3
QQGNTLPYT

SEQ ID NO: 10 - protein, 107 aa, synthetic construct
  REGION 1..107: note = Anti-CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 11 - protein, 8 aa, synthetic construct
  REGION 1..8: note = Anti-CD19 CDR-H1 (axicabtagene ciloleucel)
SGVSLPDY

SEQ ID NO: 12 - protein, 15 aa, synthetic construct
  REGION 1..15: note = Anti-CD19 CDR-H2 (axicabtagene ciloleucel)
LEWLGVIWGSETTYY

SEQ ID NO: 13 - protein, 11 aa, synthetic construct
  REGION 1..11: note = Anti-CD19 CDR-H3 (axicabtagene ciloleucel)
HYYYGGSYAMD

SEQ ID NO: 14 - protein, 117 aa, synthetic construct
  REGION 1..117: note = Anti-CD19 VH (axicabtagene ciloleucel)
KLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 15 - protein, 6 aa, synthetic construct
  REGION 1..6: note = Anti-CD19 CDR-L1 (axicabtagene ciloleucel)
QDISKY

SEQ ID NO: 16 - skipped

SEQ ID NO: 17 - protein, 9 aa, synthetic construct
  REGION 1..9: note = Anti-CD19 CDR-L3 (axicabtagene ciloleucel)
QQGNTLPYT

SEQ ID NO: 18 - protein, 120 aa, synthetic construct
  REGION 1..120: note = Anti-CD19 VL (axicabtagene ciloleucel)
CELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITG

SEQ ID NO: 19 - protein, 8 aa, synthetic construct
  REGION 1..8: note = Anti-CD19 CDR-H1 (tisagenlecleucel)
GVSLPDYG

SEQ ID NO: 20 - protein, 7 aa, synthetic construct
  REGION 1..7: note = Anti-CD19 CDR-H2 (tisagenlecleucel)
IWGSETT

SEQ ID NO: 21 - protein, 14 aa, synthetic construct
  REGION 1..14: note = Anti-CD19 CDR-H3 (tisagenlecleucel)
AKHYYYGGSYAMDY

SEQ ID NO: 22 - protein, 94 aa, synthetic construct
  REGION 1..94: note = Anti-CD19 VH (tisagenlecleucel)
GVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 23 - protein, 6 aa, synthetic construct
  REGION 1..6: note = Anti-CD19 CDR-L1 (tisagenlecleucel)
QDISKY

SEQ ID NO: 24 - skipped

SEQ ID NO: 25 - protein, 9 aa, synthetic construct
  REGION 1..9: note = Anti-CD19 CDR-L3 (tisagenlecleucel)
QQGNTLPYT

SEQ ID NO: 26 - protein, 148 aa, synthetic construct
  REGION 1..148: note = Anti-CD19 VL (tisagenlecleucel)
PDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVS

SEQ ID NO: 27 - protein, 10 aa, synthetic construct
  REGION 1..10: note = Anti-CD22 CDR-H1
GDSVSSNSAA

SEQ ID NO: 28 - protein, 9 aa, synthetic construct
  REGION 1..9: note = Anti-CD22 CDR-H2
TYYRSKWYN

SEQ ID NO: 29 - protein, 14 aa, synthetic construct
  REGION 1..14: note = Anti-CD22 CDR-H3
AREVTGDLEDAFDI

SEQ ID NO: 30 - protein, 124 aa, synthetic construct
  REGION 1..124: note = Anti-CD22 VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 31 - protein, 6 aa, synthetic construct
  REGION 1..6: note = Anti-CD22 CDR-L1
QTIWSY

SEQ ID NO: 32 - skipped

SEQ ID NO: 33 - protein, 9 aa, synthetic construct
  REGION 1..9: note = Anti-CD22 CDR-L3
QQSYSIPQT

SEQ ID NO: 34 - protein, 107 aa, synthetic construct
  REGION 1..107: note = Anti-CD22 VL
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 35 - protein, 69 aa, synthetic construct
  REGION 1..69: note = CD8-alpha Chain
TTTPAPRPPTPAPTIASQPLSLRPEACRPAAGGAVHTRGLDFACDIYIWAPLAGTCGVLLLSLVITLYC

SEQ ID NO: 36 - protein, 69 aa, synthetic construct; identical to SEQ ID NO: 35
  REGION 1..69: note = CD8-alpha Chain (tisagenlecleucel)

SEQ ID NO: 37 - protein, 42 aa, synthetic construct
  REGION 1..42: note = 4-1BB Domain
KRGRKKLLYIFKQPFMRPVQTTQEEDGCSCRFPEEEEGGCEL

SEQ ID NO: 38 - protein, 38 aa, synthetic construct
  REGION 1..38: note = 4-1BB Domain (tisagenlecleucel)
KKLLYIFKQPFMRPVQTTQEEDGCSCRFPEEEEGGCEL

SEQ ID NO: 39 - protein, 107 aa, synthetic construct
  REGION 1..107: note = CD28 Domain (axicabtagene ciloleucel)
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 40 - protein, 112 aa, synthetic construct
  REGION 1..112: note = CD3-zeta Domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 112 aa, synthetic construct
  REGION 1..112: note = CD3-zeta Domain (axicabtagene ciloleucel)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 112 aa, synthetic construct; identical to SEQ ID NO: 40
  REGION 1..112: note = CD3-zeta Domain (tisagenlecleucel)

SEQ ID NO: 43 - protein, 736 aa, synthetic construct
  REGION 1..736: note = CAR Amino Acid
SATMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 2216 bp, synthetic construct
  misc_feature 1..2216: note = CAR Nucleotide
  source 1..2216: mol_type = other DNA
ctagcgccaccatgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtc

SEQ ID NO: 45 - protein, 489 aa, synthetic construct
  REGION 1..489: note = CAR Amino Acid (axicabtagene ciloleucel)
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1470 bp, synthetic construct
  misc_feature 1..1470: note = CAR Nucleotide (axicabtagene ciloleucel)
  source 1..1470: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 47 - protein, 486 aa, synthetic construct
  REGION 1..486: note = CAR Amino Acid (tisagenlecleucel)
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 1458 bp, synthetic construct
  misc_feature 1..1458: note = CAR Nucleotide (tisagenlecleucel)
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 49 - protein, 8 aa, synthetic construct
  REGION 1..8: note = Anti-BCMA CDR-H1 (idecabtagene vicleucel)
GYTFTDYS

SEQ ID NO: 50 - protein, 8 aa, synthetic construct
  REGION 1..8: note = Anti-BCMA CDR-H2 (idecabtagene vicleucel)
INTETREP

SEQ ID NO: 51 - protein, 10 aa, synthetic construct
  REGION 1..10: note = Anti-BCMA CDR-H3 (idecabtagene vicleucel)
ALDYSYAMDY

SEQ ID NO: 52 - protein, 115 aa, synthetic construct
  REGION 1..115: note = Anti-BCMA VH (idecabtagene vicleucel)
QLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 53 - protein, 10 aa, synthetic construct
  REGION 1..10: note = Anti-BCMA CDR-L1 (idecabtagene vicleucel)
ESVTILGSHL

SEQ ID NO: 54 - skipped

SEQ ID NO: 55 - protein, 9 aa, synthetic construct
  REGION 1..9: note = Anti-BCMA CDR-L3 (idecabtagene vicleucel)
LQSRTIPRT

SEQ ID NO: 56 - protein, 109 aa, synthetic construct
  REGION 1..109: note = Anti-BCMA VL (idecabtagene vicleucel)
IVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEI

SEQ ID NO: 57 - protein, 493 aa, synthetic construct
  REGION 1..493: note = CAR Amino Acid (idecabtagene vicleucel)
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1485 bp, synthetic construct
  misc_feature 1..1485: note = CAR Nucleotide (idecabtagene vicleucel)
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 59 - protein, 8 aa, synthetic construct
  REGION 1..8: note = Anti-BCMA CDR-H1 (ciltacabtagene autoleucel)
EHTFSSHV

SEQ ID NO: 60 - protein, 8 aa, synthetic construct
  REGION 1..8: note = Anti-BCMA CDR-H2 (ciltacabtagene autoleucel)
IGWRDIST

SEQ ID NO: 61 - protein, 12 aa, synthetic construct
  REGION 1..12: note = Anti-BCMA CDR-H3 (ciltacabtagene autoleucel)
AARRIDAADFDS

SEQ ID NO: 62 - protein, 119 aa, synthetic construct
  REGION 1..119: note = Anti-BCMA VHH (ciltacabtagene autoleucel)
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 63 - protein, 5 aa, synthetic construct
  REGION 1..5: note = Anti-BCMA CDR-H1 (ciltacabtagene autoleucel)
GRTFT

SEQ ID NO: 64 - protein, 8 aa, synthetic construct
  REGION 1..8: note = Anti-BCMA CDR-H2 (ciltacabtagene autoleucel)
ISLSPTLA

SEQ ID NO: 65 - protein, 14 aa, synthetic construct
  REGION 1..14: note = Anti-BCMA CDR-H3 (ciltacabtagene autoleucel)
AADRKSVMSIRPDY

SEQ ID NO: 66 - protein, 118 aa, synthetic construct
  REGION 1..118: note = Anti-BCMA VHH (ciltacabtagene autoleucel)
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 67 - protein, 486 aa, synthetic construct
  REGION 1..486: note = CAR Amino Acid (ciltacabtagene autoleucel)
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1461 bp, synthetic construct
  misc_feature 1..1461: note = CAR Nucleotide (ciltacabtagene autoleucel)
  source 1..1461: mol_type = other DNA
atggctctgcccgtcaccgctctgctgctgcctctggctctgctgctgcacgctgctcgccctcaggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagcggaggaggaggatctgaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 69 - protein, 111 aa, synthetic construct
  REGION 1..111: note = CD8-alpha Chain (idecabtagene vicleucel)
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 70 - protein, 69 aa, synthetic construct; identical to SEQ ID NO: 35
  REGION 1..69: note = CD8-alpha Chain (ciltacabtagene autoleucel)

SEQ ID NO: 71 - protein, 42 aa, synthetic construct; identical to SEQ ID NO: 37
  REGION 1..42: note = CD137 Domain (ciltacabtagene autoleucel)

SEQ ID NO: 72 - protein, 112 aa, synthetic construct; identical to SEQ ID NO: 41
  REGION 1..112: note = CD3-zeta Domain (idecabtagene vicleucel)

SEQ ID NO: 73 - protein, 112 aa, synthetic construct; identical to SEQ ID NO: 41
  REGION 1..112: note = CD3-zeta Domain (ciltacabtagene autoleucel)